SEQUENCE LISTING (111 sequences)

SEQ ID NO: 1 - protein, 4 aa, Artificial Sequence
  Synthetic sequence ITAM motif
  VARIANT (2)...(3): Xaa = any amino acid
  VARIANT 4: Xaa = L or I
YXXX

SEQ ID NO: 2 - protein, 16 aa, Artificial Sequence
  Synthetic sequence ITAM motif
  VARIANT 2,3: Xaa = any amino acid
  VARIANT 4: Xaa = L or I
  VARIANT 5,6,7,8,9,10,11,12: Xaa = any amino acid
  Misc_Feature 11,12: Any one of amino acids 11-12 can be either present or absent
  VARIANT 14,15: Xaa = any amino acid
  VARIANT 16: Xaa = L or I
YXXXXXXXXXXXYXXX

SEQ ID NO: 3 - protein, 8 aa, Artificial Sequence
  Synthetic sequence Lck-binding motif
  VARIANT 3,4,6,7: Xaa = any amino acid
RKXXYXXY

SEQ ID NO: 4 - protein, 9 aa, Artificial Sequence
  Synthetic sequence domain within CD3epsilon ITAM
  VARIANT (3)...(8): Xaa = any amino acid
RKXXXXXXY

SEQ ID NO: 5 - protein, 185 aa, Artificial Sequence
  Synthetic sequence human CD3epsilon without signal peptide
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 6 - protein, 15 aa, Artificial Sequence
  Synthetic sequence human CD3epsilon ITAM sequence
YEPIRKGQRDLYSGL

SEQ ID NO: 7 - protein, 7 aa, Artificial Sequence
  Synthetic sequence human CD3epsilon PRS
PPPVPNP

SEQ ID NO: 8 - protein, 23 aa, Artificial Sequence
  Synthetic sequence human CD3epsilon fragment
PPPVPNPDYEPIRKGQRDLYSGL

SEQ ID NO: 9 - protein, 8 aa, Artificial Sequence
  Synthetic sequence human CD3epsilon BRS
KNRKAKAK

SEQ ID NO: 10 - protein, 160 aa, Artificial Sequence
  Synthetic sequence human CD3gamma without signal peptide
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 11 - protein, 45 aa, Artificial Sequence
  Synthetic sequence human CD3gamma cytoplasmic domain sequence
GQDGVRQSRASDKQTLLPNDQLYQPLKDREDDQYSHLQGNQLRRN

SEQ ID NO: 12 - protein, 15 aa, Artificial Sequence
  Synthetic sequence human CD3gamma ITAM sequence
YQPLKDREDDQYSHL

SEQ ID NO: 13 - protein, 149 aa, Artificial Sequence
  Synthetic sequence human CD3delta without signal peptide
KIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 14 - protein, 45 aa, Artificial Sequence
  Synthetic sequence human CD3delta cytoplasmic domain
GHETGRLSGAADTQALLRNDQVYQPLRDRDDAQYSHLGGNWARNK

SEQ ID NO: 15 - protein, 15 aa, Artificial Sequence
  Synthetic sequence human CD3delta ITAM sequence
YQPLRDRDDAQYSHL

SEQ ID NO: 16 - protein, 143 aa, Artificial Sequence
  Synthetic sequence human CD3zeta without signal peptide
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 113 aa, Artificial Sequence
  Synthetic sequence human CD3zeta cytoplasmic domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 15 aa, Artificial Sequence
  Synthetic human CD3zeta ITAM (a) sequence
YNELNLGRREEYDVL

SEQ ID NO: 19 - protein, 16 aa, Artificial Sequence
  Synthetic human CD3zeta ITAM (b) sequence
YNELQKDKMAEAYSEI

SEQ ID NO: 20 - protein, 15 aa, Artificial Sequence
  Synthetic human CD3zeta ITAM (c) sequence
YQGLSTATKDTYDAL

SEQ ID NO: 21 - protein, 5 aa, Artificial Sequence
  Synthetic human CD3zeta BRS (a) sequence
KRRGR

SEQ ID NO: 22 - protein, 6 aa, Artificial Sequence
  Synthetic human CD3zeta BRS (b) sequence
KPQRRK

SEQ ID NO: 23 - protein, 8 aa, Artificial Sequence
  Synthetic human CD3zeta BRS (c) sequence
KGERRRGK

SEQ ID NO: 24 - protein, 29 aa, Artificial Sequence
  Synthetic sequence comprising human CD3epsilon PRS and ITAM
RPPPVPNPDYEPIRKGQRDLYSGLNQRRI

SEQ ID NO: 25 - protein, 43 aa, Artificial Sequence
  Synthetic human 4-1BB signaling sequence
VKRGRKKLLYIFKQPFMRPVQTTQEEDGCSCRFPEEEEGGCEL

SEQ ID NO: 26 - protein, 28 aa, Artificial Sequence
  Synthetic human CD28 transmembrane sequence
MFWVLVVVGGVLACYSLLVTVAFIIFWV

SEQ ID NO: 27 - protein, 167 aa, Artificial Sequence
  Synthetic CD3epsilon_CD3zeta sequence
KNRKAKAKPVTRGAGAGGRQRGQNKERPPPVPNPDYEPIRKGQRDLYSGLNQRRIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 167 aa, Artificial Sequence
  Synthetic CD3zeta_CD3epsilon sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKNRKAKAKPVTRGAGAGGRQRGQNKERPPPVPNPDYEPIRKGQRDLYSGLNQRRI

SEQ ID NO: 29 - protein, 141 aa, Artificial Sequence
  Synthetic CD3epsilon_PRS_ITAM CD3zeta sequence
RPPPVPNPDYEPIRKGQRDLYSGLNQRRIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 209 aa, Artificial Sequence
  Synthetic 4-1BB_CD3epsilon_ CD3zeta sequence
KRGRKKLLYIFKQPFMRPVQTTQEEDGCSCRFPEEEEGGCELKNRKAKAKPVTRGAGAGGRQRGQNKERPPPVPNPDYEPIRKGQRDLYSGLNQRRIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 209 aa, Artificial Sequence
  Synthetic 4-1BB_CD3zeta _ CD3epsilon sequence
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKNRKAKAKPVTRGAGAGGRQRGQNKERPPPVPNPDYEPIRKGQRDLYSGLNQRRI

SEQ ID NO: 32 - protein, 183 aa, Artificial Sequence
  Synthetic 4-1BB_CD3epsilon_PRS_ITAM CD3zeta sequence
KRGRKKLLYIFKQPFMRPVQTTQEEDGCSCRFPEEEEGGCELRPPPVPNPDYEPIRKGQRDLYSGLNQRRIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 12 aa, Artificial Sequence
  Synthetic IgG4 hinge sequence
  VARIANT 10: Xaa = P or S
ESKYGPPCPXCP

SEQ ID NO: 34 - DNA, 165 bp, Artificial Sequence
  Synthetic CD3epsilon intracellular sequence
aagaaccggaaggccaaggccaagcctgtgacaagaggtgctggtgctggcggcagacagagaggccagaacaaagaaagacctcctcctgtgcctaatcctgactacgagcccatccggaagggccagagagatctgtactccggcctgaaccagcggcggatt

SEQ ID NO: 35 - DNA, 87 bp, Artificial Sequence
  Synthetic CD3epsilon_PRS_ITAM CD3zeta sequence
agacctcctcctgtgcctaatcctgactacgagcccatccggaagggccagagagatctgtactccggcctgaaccagcggcggatt

SEQ ID NO: 36 - DNA, 336 bp, Artificial Sequence
  Synthetic CD3zeta intracellular sequence
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 37 - DNA, 126 bp, Artificial Sequence
  Synthetic 4-1BB signaling sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 38 - DNA, 84 bp, Artificial Sequence
  Synthetic CD28 transmembrane sequence
atgttctgggtgctggtggtggtcggaggcgtgctggcctgctacagcctgctggtcaccgtggccttcatcatcttttgggtg

SEQ ID NO: 39 - DNA, 36 bp, Artificial Sequence
  Synthetic IgG4 hinge sequence
gagagcaagtacggaccgccctgccccccttgccct

SEQ ID NO: 40 - DNA, 633 bp, Artificial Sequence
  Synthetic CD28tm_4-1BB signal_CD3epsilon_PRS_ITAM CD3zeta sequence
atgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgagacctcctcctgtgcctaatcctgactacgagcccatccggaagggccagagagatctgtactccggcctgaaccagcggcggattcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 41 - DNA, 711 bp, Artificial Sequence
  Synthetic CD28tm_4-1BB signal_CD3epsilon_CD3zeta sequence
atgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgaagaaccggaaggccaaggccaagcctgtgacaagaggtgctggtgctggcggcagacagagaggccagaacaaagaaagacctcctcctgtgcctaatcctgactacgagcccatccggaagggccagagagatctgtactccggcctgaaccagcggcggattcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 42 - DNA, 711 bp, Artificial Sequence
  Synthetic CD28tm_4-1BB signal_CD3zeta_CD3epsilon sequence
atgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggaagaaccggaaggccaaggccaagcctgtgacaagaggtgctggtgctggcggcagacagagaggccagaacaaagaaagacctcctcctgtgcctaatcctgactacgagcccatccggaagggccagagagatctgtactccggcctgaaccagcggcggatt

SEQ ID NO: 43 - DNA, 2673 bp, Artificial Sequence
  Synthetic construct sequence GM-CSF leader_FMC63 scFv_STII_(G4S)2_IgG4 hinge_CD28tm_4-1BB_CD3epsilon_PRS_ITAM CD3zeta_T2A_GM-CSF leader_tEGFR
atgctgctgctggtgaccagcctgctgctgtgcgagctgccccaccctgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgagacctcctcctgtgcctaatcctgactacgagcccatccggaagggccagagagatctgtactccggcctgaaccagcggcggattcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 44 - DNA, 2682 bp, Artificial Sequence
  Synthetic construct sequence GM-CSF leader_R12 scFv_STII_(G4S)2_IgG4 hinge_CD28tm_4-1BB_CD3epsilon_PRS_ITAM CD3zeta_T2A_GM-CSF leader_tEGFR
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgagacctcctcctgtgcctaatcctgactacgagcccatccggaagggccagagagatctgtactccggcctgaaccagcggcggattcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 45 - DNA, 2760 bp, Artificial Sequence
  Synthetic construct sequence GM-CSF leader_R12 scFv_STII_(G4S)2_IgG4 hinge_CD28tm_4-1BB_CD3epsilon_CD3zeta_T2A_GM-CSF leader_tEGFR
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgaagaaccggaaggccaaggccaagcctgtgacaagaggtgctggtgctggcggcagacagagaggccagaacaaagaaagacctcctcctgtgcctaatcctgactacgagcccatccggaagggccagagagatctgtactccggcctgaaccagcggcggattcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 46 - DNA, 2760 bp, Artificial Sequence
  Synthetic construct sequence GM-CSF leader_R12 scFv_STII_(G4S)2_IgG4 hinge_CD28tm_4-1BB_CD3zeta_CD3epsilon_T2A_GM-CSF leader_tEGFR
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggaagaaccggaaggccaaggccaagcctgtgacaagaggtgctggtgctggcggcagacagagaggccagaacaaagaaagacctcctcctgtgcctaatcctgactacgagcccatccggaagggccagagagatctgtactccggcctgaaccagcggcggattctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 47 - protein, 41 aa, Artificial Sequence
  Synthetic CD28 intracellular amino acid sequence (LL to GG)
RSKRSRGGHSDYMNMTPRRPGPTRKHYQPYAPPRDFAAYRS

SEQ ID NO: 48 - protein, 42 aa, Artificial Sequence
  Synthetic OX40 intracellular amino acid sequence
ALYLLRRDQRLPPDAHKPPGGGSFRTPIQEEQADAHSTLAKI

SEQ ID NO: 49 - protein, 38 aa, Artificial Sequence
  Synthetic ICOS intracellular amino acid sequence
CWLTKKKYSSSVHDPNGEYMFMRAVNTAKKSRLTDVTL

SEQ ID NO: 50 - protein, 22 aa, Artificial Sequence
  Synthetic Porcine teschovirus-1 2A (P2A) peptide
GSGATNFSLLKQAGDVEENPGP

SEQ ID NO: 51 - protein, 24 aa, Artificial Sequence
  Synthetic Thoseaasigna virus 2A (T2A) peptide
LEGGGEGRGSLLTCGDVEENPGPR

SEQ ID NO: 52 - protein, 20 aa, Artificial Sequence
  Synthetic Equine rhinitis A virus (ERAV) 2A (E2A) peptide
QCTNYALLKLAGDVESNPGP

SEQ ID NO: 53 - protein, 25 aa, Artificial Sequence
  Synthetic Foot-and-Mouth disease virus 2A (F2A) peptide
GSGVKQTLNFDLLKLAGDVESNPGP

SEQ ID NO: 54 - protein, 335 aa, Artificial Sequence
  Synthetic tEGFR sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 55 - protein, 248 aa, Artificial Sequence
  Synthetic anti-ROR1 scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 56 - protein, 5 aa, Artificial Sequence
  Synthetic R11 ANTIBODY CDRH1
DYPIS

SEQ ID NO: 57 - protein, 16 aa, Artificial Sequence
  Synthetic R11 ANTIBODY CDRH2
FINSGGSTWYASWVKG

SEQ ID NO: 58 - protein, 11 aa, Artificial Sequence
  Synthetic R11 ANTIBODY CDRH3
GYSTYYGDFNI

SEQ ID NO: 59 - protein, 11 aa, Artificial Sequence
  Synthetic R11 ANTIBODY CDRL1
QASQSIDSNLA

SEQ ID NO: 60 - protein, 7 aa, Artificial Sequence
  Synthetic R11 ANTIBODY CDRL2
RASNLAS

SEQ ID NO: 61 - protein, 12 aa, Artificial Sequence
  Synthetic R11 ANTIBODY CDRL3
LGGVGNVSYRTS

SEQ ID NO: 62 - protein, 116 aa, Artificial Sequence
  Synthetic R11 ANTIBODY VH
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 63 - protein, 110 aa, Artificial Sequence
  Synthetic R11 ANTIBODY VL
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 64 - protein, 241 aa, Artificial Sequence
  Synthetic R11 SCFV
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 65 - protein, 5 aa, Artificial Sequence
  Synthetic Y31 ANTIBODY CD
SHWMS

SEQ ID NO: 66 - protein, 16 aa, Artificial Sequence
  Synthetic Y31 ANTIBODY CDRH2
IIAASGSTYYANWAKG

SEQ ID NO: 67 - protein, 12 aa, Artificial Sequence
  Synthetic Y31 ANTIBODY CDRH3
DYGDYRLVTFNI

SEQ ID NO: 68 - protein, 11 aa, Artificial Sequence
  Synthetic Y31 ANTIBODY CDRL1
QASQSIGSYLA

SEQ ID NO: 69 - protein, 7 aa, Artificial Sequence
  Synthetic Y31 ANTIBODY CDRL2
YASNLAS

SEQ ID NO: 70 - protein, 9 aa, Artificial Sequence
  Synthetic Y31 ANTIBODY CDRL3
LGSLNSDNV

SEQ ID NO: 71 - protein, 5 aa, Artificial Sequence
  Synthetic 2A2 ANTIBODY CDRH1
DYEMH

SEQ ID NO: 72 - protein, 17 aa, Artificial Sequence
  Synthetic 2A2 ANTIBODY CDRH2
AIDPETGGTAYNQKFKG

SEQ ID NO: 73 - protein, 9 aa, Artificial Sequence
  Synthetic 2A2 ANTIBODY CDRH3
YYDYDSFTY

SEQ ID NO: 74 - protein, 11 aa, Artificial Sequence
  Synthetic 2A2 ANTIBODY CDRL1
KASQNVDAAVA

SEQ ID NO: 75 - protein, 7 aa, Artificial Sequence
  Synthetic 2A2 ANTIBODY CDRL2
SASNRYT

SEQ ID NO: 76 - protein, 9 aa, Artificial Sequence
  Synthetic 2A2 ANTIBODY CDRL3
QQYDIYPYT

SEQ ID NO: 77 - protein, 118 aa, Artificial Sequence
  Synthetic 2A2 ANTIBODY VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 78 - protein, 107 aa, Artificial Sequence
  Synthetic 2A2 ANTIBODY VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 79 - protein, 237 aa, Artificial Sequence
  Synthetic 2A2 SCFV
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGSGGGSGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 80 - protein, 5 aa, Artificial Sequence
  Synthetic R12 ANTIBODY CDRH1
AYYMS

SEQ ID NO: 81 - protein, 17 aa, Artificial Sequence
  Synthetic R12 ANTIBODY CDRH2
TIYPSSGKTYYATWVNG

SEQ ID NO: 82 - protein, 12 aa, Artificial Sequence
  Synthetic R12 ANTIBODY CDRH3
DSYADDGALFNI

SEQ ID NO: 83 - protein, 12 aa, Artificial Sequence
  Synthetic R12 ANTIBODY CDRL1
TLSSAHKTDTID

SEQ ID NO: 84 - protein, 7 aa, Artificial Sequence
  Synthetic R12 ANTIBODY CDRL2
GSYTKRP

SEQ ID NO: 85 - protein, 9 aa, Artificial Sequence
  Synthetic R12 ANTIBODY CDRL3
GADYIGGYV

SEQ ID NO: 86 - protein, 121 aa, Artificial Sequence
  Synthetic R12 ANTIBODY VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 87 - protein, 112 aa, Artificial Sequence
  Synthetic R12 ANTIBODY VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 88 - protein, 6 aa, Artificial Sequence
  Synthetic ANTI-ROR1 SCFV CDRH1
DTIDWY

SEQ ID NO: 89 - protein, 16 aa, Artificial Sequence
  Synthetic ANTI-ROR1 SCFV CDRH2
VQSDGSYTKRPGVPDR

SEQ ID NO: 90 - protein, 8 aa, Artificial Sequence
  Synthetic ANTI-ROR1 SCFV CDRH3
YIGGYVFG

SEQ ID NO: 91 - protein, 11 aa, Artificial Sequence
  Synthetic ANTI-ROR1 SCFV CDRL1
ASGFDFSAYYM

SEQ ID NO: 92 - protein, 7 aa, Artificial Sequence
  Synthetic ANTI-ROR1 SCFV CDRL2
TIYPSSG

SEQ ID NO: 93 - protein, 9 aa, Artificial Sequence
  Synthetic ANTI-ROR1 SCFV CDRL3
ADRATYFCA

SEQ ID NO: 94 - protein, 5 aa, Artificial Sequence
  Synthetic ANTI-ROR1 SCFV CDRH1
SNLAW

SEQ ID NO: 95 - protein, 16 aa, Artificial Sequence
  Synthetic ANTI-ROR1 SCFV CDRH2
RASNLASGVPSRFSGS

SEQ ID NO: 96 - protein, 8 aa, Artificial Sequence
  Synthetic ANTI-ROR1 SCFV CDRH3
NVSYRTSF

SEQ ID NO: 97 - protein, 11 aa, Artificial Sequence
  Synthetic ANTI-ROR1 SCFV CDRL1
SGSDINDYPIS

SEQ ID NO: 98 - protein, 7 aa, Artificial Sequence
  Synthetic ANTI-ROR1 SCFV CDRL2
INSGGST

SEQ ID NO: 99 - protein, 8 aa, Artificial Sequence
  Synthetic ANTI-ROR1 SCFV CDRL3
YFCARGYS

SEQ ID NO: 100 - protein, 107 aa, Artificial Sequence
  Synthetic ANTI-CD19 FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 101 - protein, 120 aa, Artificial Sequence
  Synthetic ANTI-CD19 FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 102 - protein, 245 aa, Artificial Sequence
  Synthetic ANTI-CD19 FMC63 SCFV
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 103 - protein, 18 aa, Artificial Sequence
  Synthetic linker 218 amino acid sequence
GSTSGSGKPGSGEGSTKG

SEQ ID NO: 104 - protein, 10 aa, Artificial Sequence
  Synthetic linker amino acid sequence
GSGKPGSGEG

SEQ ID NO: 105 - protein, 8 aa, Artificial Sequence
  Synthetic linker amino acid sequence
GKPGSGEG

SEQ ID NO: 106 - protein, 8 aa, Artificial Sequence
  Synthetic linker amino acid sequence
SGKPGSGE

SEQ ID NO: 107 - protein, 6 aa, Artificial Sequence
  Synthetic linker amino acid sequence
  VARIANT 1: Xaa = positively charged amino acid
  VARIANT 3,4,5: Xaa = Gly or Ser
  VARIANT 6: Xaa = Gly or negatively charged amino acid
XPXXXX

SEQ ID NO: 108 - protein, 200 aa, Artificial Sequence
  Synthetic linker amino acid sequence
  VARIANT (1)...(200): any one or all of amino acids 2-10, 12-200 can either be present or absent
GGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSS

SEQ ID NO: 109 - protein, 15 aa, Artificial Sequence
  Synthetic linker amino acid sequence
GGGGSGGGGSGGGGS

SEQ ID NO: 110 - protein, 18 aa, Artificial Sequence
  Synthetic linker amino acid sequence
GSTSGGGSGGGSGGGGSS

SEQ ID NO: 111 - protein, 55 aa, Artificial Sequence
  Synthetic human CD3epsilon cytoplasmic domain
KNRKAKAKPVTRGAGAGGRQRGQNKERPPPVPNPDYEPIRKGQRDLYSGLNQRRI

(19) United States
(12) Patent Application Publication
(10) Pub. No.: US 2024/0165232 A1
(43) Pub. Date: May 23, 2024

(54) CHIMERIC RECEPTOR PROTEINS AND USES THEREOF

(71) Applicant: FRED HUTCHINSON CANCER RESEARCH CENTER, Seattle, WA (US)

(72) Inventors: Alexander SALTER, Seattle, WA (US); Stanley R. RIDDELL, Sammamish, WA (US)

(21) Appl. No.: 17/279,561

(22) PCT Filed: Sep. 23, 2019
(86) PCT No.: PCT/US2019/052492

§ 371 Date: Mar. 24, 2021

Related U.S. Application Data
(60) Provisional application No. 62/735,702, filed on Sep. 24, 2018.
(60) Provisional application No. 62/901,194, filed on Sep. 16, 2019.

(51) Int. Cl. A61K 39/00; A61P 35/00; C07K 14/705; C07K 14/715; C07K 14/725; C07K 16/28; C12N 5/0783

(52) U.S. Cl. CPC A61K 39/464412; A61K 39/4611; A61K 39/4631; A61K 39/464411; A61P 35/00; C07K 14/7051; C07K 14/70521; C07K 14/7151; C07K 16/2803; C12N 5/0636; C07K 2317/622; C07K 2319/03; C07K 2319/30; C12N 2510/00

(57) ABSTRACT

The present disclosure provides fusion proteins with improved signaling properties. Disclosed embodiments include fusion proteins that comprise an extracellular antigen-binding domain, a transmembrane domain, and an intracellular component comprising one or more domain or motif from a CD3ε, γ, or δ protein, and have improved signaling in response to antigen-binding, including of antigens with reduced, low, or intermediate levels of expression on a target cell, such as a solid tumor cell. Recombinant host cells expressing the fusion proteins are also provided, as well as compositions and methods comprising the same.

### STATEMENT OF GOVERNMENT INTEREST

This invention was made with government support under CA136551 and CA114536 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_465WO_SEQUENCE_LISTING.txt. The text file is 72.4 KB, was created on Sep. 23, 2019, and is being submitted electronically via EFS-Web.

### BACKGROUND

Adoptive transfer of genetically modified T cells has emerged as a potent therapy for various malignancies. The most widely employed strategy has been infusion of patient-derived T cells expressing chimeric antigen receptors (CARs) targeting tumor associated antigens. This approach has numerous theoretical advantages, including the ability to target T cells to any cell surface antigen, circumvent loss of major histocompatibility complex as a tumor escape mechanism, and employ a single vector construct to treat any patient, regardless of human leukocyte antigen haplotype. For example, CAR clinical trials for B-cell non-Hodgkin's lymphoma (NHL) have, to date, targeted CD19, CD20, or CD22 antigens that are expressed on malignant lymphoid cells as well as on normal B cells (Brentjens et al., Sci Transl Med 2013; 5(177):177ra38; Haso et al., Blood 2013; 121(7):1165-74; James et al., J Immunol 2008; 180(10):7028-38; Kalos et al., Sci Transl Med 2011; 3(95):95ra73; Kochenderfer et al., J Clin Oncol 2015; 33(6):540-9; Lee et al., Lancet 2015; 385(9967):517-28; Porter et al., Sci Transl 25 Med 2015; 7(303):303ra139; Savoldo et al., J Clin Invest 2011; 121(5):1822-6; Till et al., Blood 2008; 112(6):2261-71; Till et al., Blood 2012; 119(17):3940-50; Coiffier et al., N Engl J Med 2002; 346(4):235-42).

However, adoptive cell therapies are still developing, and new strategies are needed, for example, to improve CAR functionality when targeting antigens in vivo, including in the context of solid tumors. The presently disclosed embodiments address these needs and provide other related advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1N show design and testing of bi-specific T cells for selective analysis of TCR- and CAR-induced signaling and effector functions in a single-cell-type population. (A) Schematic of bi-specific T cells that express an endogenous EBV-specific T cell receptor (TCR) and were transduced to express a ROR1-specific chimeric antigen receptor (CAR). (B) Flow cytometry analysis of EBV-tetramer binding and CD19t expression in expanded T cells. FACS plot shows stained (black) and isotype (grey) CD8⁺ singlet lymphocytes. (C) Schematic of magnetic beads coated with HLA-B8/EBV-RAK single chain trimer (SCT), ROR1 ectodomain, or SCT and CD28 mAb. (D) Western blot analysis for CD3ζ, CD3ζ pTyr¹⁴², and PLC-γ1 pTyr⁷⁸³ in lysates from T cells incubated for 45 minutes with microbeads coated with various molar ratios of SCT or ROR1. 30 μL beads were used per million cells. (E) Western blot analysis for CD3ζ, CD3ζ pTyr¹⁴², and PLC-γ1 pTyr⁷⁸³ in lysates from T cells incubated for 45 minutes with various amounts SCT or ROR1 microbeads (molar ratio=0.5). (F) Western blot analysis for CD3ζ, CD3ζ pTyr¹⁴², and PLC-γ1 pTyr⁷⁸³ in lysates from T cells incubated for 45 minutes with uncoated (0), SCT, or SCT/CD28 microbeads. 7.5 μL beads were used per million cells. (G) Flow cytometry analysis of T cell proliferation as measured by CFSE dye dilution at 72 hours after microbead stimulation. Histogram plot of CD8⁺ lymphocytes treated with uncoated (dash-dot), SCT (dashed line) or SCT/CD28 (solid line) microbeads. (H) Flow cytometry analysis of T cell proliferation as measured by CFSE dye dilution at 72 hours after co-culture with: K562/B8 cells or control beads; K562/B8/EBV cells or SCT/CD28 beads; or K562/B8/ROR1⁺ cells or ROR1-coated beads. Histogram plots show CD8⁺ lymphocytes and are representative of 2-3 independent experiments. (I)-(L) Flow cytometry analysis showing IFN-γ, IL-2, and TNF-α production 5 hours after co-culture of bi-specific T cells with: K562/B8 cells; K562/B8/EBV cells; K562/B8/ROR1⁺ cells; control beads; SCT/CD28 beads; or ROR1-coated beads. (I, K) FACS plots show CD45⁺ singlet lymphocytes. The frequency of IFN-γ⁺IL-2⁺TNFα⁺ cells is shown in (I). Data are means±standard deviation (SD) of 2-3 independent experiments. The indicated P values were calculated by unpaired two-tailed t test. Cytokine production is quantified in FIGS. 1J and 1L. (M) Western blot analysis for CD3ζ, CD3ζ pTyr¹⁴², SLP-76 pSer³⁷⁶, and PLC-γ1 pTyr⁷⁸³ in T cell lysates after stimulation with beads or cells for 45 minutes. (N) Ca2⁺ mobilization in populations of cells stimulated by ROR1- or SCT-containing bilayers was measured by Fluo-4 AM intensity of individual cell responses for 15 min after exposure to bilayers. Traces represent the fraction of cells above an activation threshold at any given time. Antigen density was modulated by the mole fraction of biotinylated lipids in the supported lipid bilayer—0.005% or 0.01%.

FIGS. 2A-2M relate to experiments identifying protein phosphorylation events by mass spectrometry (MS) following TCR or CAR stimulation. (A) Schematic of experimental design stimulating bi-specific T cells with microbeads coated with TCR (SCT/CD28) or CAR (ROR1) antigens. (B) Flow cytometry analysis of T cell markers in the three experiments. (C) Venn diagram of the overlap among phosphorylation sites. (D) Histogram of the SD of the fold-change (log 2FC) values across all tandem MS/MS experiments. (E) Total number of pSer, pThr and pTyr peptides identified by MS/MS. (F)-(H) Volcano plots of log 2FC and false discovery rate (FDR) for phosphorylation sites identified by tandem MS/MS at the indicated timepoints after stimulation. Dots in the upper right portion indicate sites with increased phosphorylation, and dots in the upper left portion indicate sites with decreased phosphorylation after stimulation in at least two experiments. (I)-(L) Fold change of the indicated PO₄ sites identified by tandem MS/MS (CD3ζ PO₄; CD28 PO₄; ZAP-70 PO₄; PLC-γ1 PO₄). Data are means from 2 or 3 experiments. (M) Western blot analysis for CD3ζ, CD3ζ pTyr¹⁴², SLP-76 pSer³⁷⁶, and PLC-γ1 pTyr⁷⁸³ in bi-specific T cell lysates at the indicated times after stimulation. Bi-specific T cells expressed a CD28/CD3ζ CAR (M). Blots are representative of all tandem MS/MS experiments.

FIGS. 3A-3H provide data from MS/MS experiments showing that CAR stimulation induces less intense PO₄ of CD3 chains and signaling adaptors as compared to TCR stimulation. (A)-(C) Comparison of the log 2FC of PO₄ sites identified by tandem MS/MS at the indicated timepoints after TCR or CAR stimulation. Dots above and below dashed lines specify sites that possessed mean log 2FC values differing by ≥1 between TCR- and CAR-stimulated samples in at least 2 tandem MS/MS experiments. Dots above and below dashed lines specify sites that possessed mean log 2FC values differing by ≥1 between TCR- and CAR-stimulated samples. (D) Heat map shows mean log 2FC values of select genes identified in (A) at the 10-minute time point. Data are means from 2 or 3 experiments. (E)-(H) Mean log 2FC of PO₄ sites identified by tandem MS/MS after TCR or CAR stimulation. Blots are representative of 2 independent experiments. Data are means from all experiments.

FIGS. 4A-4R show that T cells expressing CARs that incorporate CD3ε ITAM and protein recruitment sequences display improved recognition of tumor cells with low antigen density. (A) Schematic of CD3ε-containing CAR designs that included (not shown) a CD19-specific scFv derived from FMC63 or (shown) a ROR1-specific scFv derived from R12 antibody. (B) Western blot analysis for CD3ζ in the indicated CAR T cell lysates. Blots are representative of 2 independent experiments. (C) Flow cytometric analysis of EGFRt and STII (CAR) expression on sort purified and expanded CD8⁺EGFRt⁺ T cells. (D) Western blot analysis for PLC-γ1 pTyr⁷⁸³, SLP-76 pSer³⁷⁶, and CAR and TCR CD3ζ in the indicated CAR T cell lysates following stimulation (“+”) or not (“−”). (E) Flow cytometry analysis of CD19 expression on K562 cells with low (“K562/CD19^{lo}”) or high (“K562/CD19^{hi}”) expression. FACS plot shows isotype (dash-dot), K562/CD19^{lo} (dashed line), and K562/CD19^{hi} (solid line) singlets. (F, G) ELISA analysis of cytokine production 24 hours after co-culture of the indicated CD19-specific CAR T cells and tumor cells having expressing antigen as indicated. Data are means from two independent experiments. (H) Mean+SEM fold-change in cytokine production in cellular supernatant 24 hours after co-culture of CAR T cells with K562/ROR1 tumor cell lines. In (F)-(H), fold change is relative to a control CAR containing the same scFv as the tested CAR, a 4-1BB costimulatory domain, and a CD3ζ domain, but not a CD3ε domain. N=4 healthy T cell donors. (I) ELISA analysis of cytokine production by the indicated ROR1-specific CAR T cells 24 hours after co-culture with tumor cells having medium surface expression of ROR1. Data are means from one experiment. (J), (K) Flow cytometry analysis of CAR T cell proliferation as measured by CFSE dye dilution at 72 hours after co-culture with the indicated tumor cells having medium (“ROR1^{med} NCI-H358”) or low (“ROR1^{lo} MDA-MB-231”) surface expression of ROR1. Histogram plots show 4-1BB/CD3ζ(solid line) and 4-1BB/CD3εPRS_ITAM/CD3ζ (dashed line) CD8⁺ lymphocytes and are representative of one experiment. (L) Fluo-4 Ca²⁺ mobilization measurements for cells stimulated on ROR1-labeled bilayers. (Left) traces represent the fraction of cells with high levels of intracellular Ca²⁺ across time after exposure to bilayers. Data from 4 independent experiments are accumulated for each trace, and fill represents the SD between experiments. (Right) Fraction of cells responding at 20 min after exposure to bilayer for a range of ROR1 densities, determined by the mol % of biotinylated lipids in the bilayer. Each data point represents 3-4 independent experiments. Error bars are SEM. (M) Mean±SEM radiance of luciferase-expressing ROR1^{low} MDA-MB-231 tumors over time. (N) (left) Mean±SEM tumor radiance at day 27 post-tumor infection and (right) CAR T cell frequency in tumors at day 20. n=7 mice per group. (O) Western blot analysis for CD3ζ, CD3ζ pTyr¹², LAT pTyr¹⁹¹, SLP-76 pSer³⁷⁶, and PLC-γ1 pTyr⁷⁸³ in T cell lysates at after 10 minutes of stimulation. Data are representative of n=3 independent experiments. (P) Survival curves of NSG mice that were injected with Raji/ffluc cells and, 7 days following engraftment, received with a single infusion of the indicated CAR T cells. Data were pooled from two independent experiments, with 6 or 10 mice per treatment group. (Q) Survival curves of NSG mice injected with ROR1^{int} Jeko-1 cells and administered CD28/CD3ζ, 4-1BB/CD3ζ, or 4-1BB/CD3ε_{PRS_ITAM}/CD3ζ CAR T cells. N=8 mice per group. (R) ROR1 expression (solid line) by MDA-MB-231 breast adenocarcinoma and NCI-H358 lung adenocarcinoma cell lines.

FIG. 5 shows characterization of ROR1 expression on various tumor cell lines. Flow cytometry analysis of ROR1 expression on the indicated tumor lines. Histograms show stained (solid line) and isotype (dashed line) singlets.

### DETAILED DESCRIPTION

The present disclosure provides chimeric receptor proteins with improved signaling properties and/or activities over existing immunoreceptor proteins (e.g., chimeric antigen receptors (CARs), T cell receptors (TCRs), or the like), which improved properties include, in certain embodiments, initiating, generating, propagating, and/or amplifying a signal in a host cell (e.g., an immune cell such as a T cell) expressing the fusion protein when the fusion protein binds to an antigen. In certain embodiments, the antigen bound by the fusion protein is expressed at a low level, or at an intermediate level, or in some embodiments, at a high level, by a target antigen-expressing cell, such as, for example, a cancer cell. Exemplary fusion proteins of this disclosure comprise (a) an extracellular component comprising a binding domain that specifically binds to an antigen; (b) a transmembrane domain (e.g., connecting the extracellular component and intracellular component); and (c) an intracellular component comprising an effector domain or a functional portion or variant thereof, wherein the effector domain or functional portion thereof comprises: (i) an Intracellular Tyrosine-based Activation Motif (ITAM) from CD3ε, or a functional portion or variant thereof; (ii) an ITAM from CD3γ, or a functional portion or variant thereof, (iii) an ITAM from CD3δ, or a functional portion or variant thereof, (iv) a Proline Rich Sequence (PRS) from CD3ε, or a functional portion or variant thereof; (v) a Basic Residue Sequence (BRS) from CD3ε and/or CD3ζ, or a functional portion or variant thereof; or (vi) any combination of (i)-(v).

In certain embodiments, a fusion protein is provided that comprises: (a) an extracellular component comprising a binding domain that specifically binds to an antigen; (b) a transmembrane domain; and (c) an intracellular component comprising an effector domain or a functional portion thereof, wherein the effector domain or functional portion thereof comprises: (i) an Intracellular Tyrosine-based Activation Motif (ITAM) from CD3ε, or a functional variant thereof (i.e., a functional variant of the ITAM); (ii) an ITAM from CD3γ, or a functional variant thereof; (iii) an ITAM from CD3δ, or a functional variant thereof; (iv) a Proline Rich Sequence (PRS) from CD3ε, or a functional variant thereof; (v) a Basic Residue Sequence (BRS) from CD3ε and/or CD3ζ, or a functional variant thereof; or (vi) any combination of (i)-(v), and does not comprise an ectodomain or a transmembrane domain, or a portion thereof, from CD3ε, CD3γ and/or CD3δ.

In certain embodiments, an effector domain or a functional portion or variant thereof from a CD3ε is not a full-length CD3ε and is not an endodomain (also referred to herein as a cytoplasmic domain) from CD3ε.

In certain embodiments, an intracellular component of a fusion protein comprises a PRS and an ITAM from CD3ε.

In some embodiments, a fusion protein does not comprise a BRS from CD3ε, or a functional variant thereof. In other embodiments, the effector domain of the fusion protein comprises a BRS from CD3ε, or a functional variant thereof (i.e., of the BRS).

In certain embodiments, the intracellular component further comprises an ITAM from CD3ζ, or a functional variant thereof (i.e., of the ITAM). In some embodiments, the intracellular component comprises a cytoplasmic domain (also referred to as an endodomain) from a human CD3ζ, or a functional portion or variant thereof.

In certain embodiments, the intracellular component further comprises a costimulatory domain or a functional portion or variant thereof (e.g., from 4-1BB).

In some embodiments, the intracellular component comprises a costimulatory domain, an endodomain or effector domain from CD3ζ, or a functional portion or variant thereof, and an effector domain from CD3ε, or a functional portion or variant thereof. In further embodiments, the effector domain from CD3ε (or functional portion or variant of an effector domain from CD3ε) is disposed between the costimulatory domain (or functional portion or variant of the costimulatory domain) and the endodomain or effector domain from CD3ζ(or functional portion or variant of the endodomain or effector domain from CD3ζ). In other embodiments, the endodomain or effector domain from CD3ζ or functional portion or variant thereof is disposed between the costimulatory domain and the effector domain from CD3ε or functional portion or variant thereof.

In certain embodiments, a fusion protein of the present disclosure does not comprise a TCR extracellular domain, preferably does not comprise a TCR ectodomain.

In certain embodiments, a fusion protein of the present disclosure, when expressed by a T cell, does not associate with or form a TCR complex (e.g., a TCR complex as described herein, which can include one or more endogenous or heterologous components).

In certain embodiments, the extracellular component comprises a CH1, a CH2, a CH3, a CL (i.e., from an immunoglobulin), or comprises an amino acid sequence derived therefrom, a CD8 extracellular domain, a CD4 extracellular domain, a CD28 extracellular domain (e.g., amino acids 19-152 of the CD28 amino acid sequence provided in UniProt entry P10747) or a functional variant or portion thereof, or comprises a combination thereof.

The presently disclosed fusion proteins can be useful in cellular immunotherapies comprising host cells (e.g., an immune system cell such as, for example, a T cell, NK cell, or NK-T cell, or a hematopoietic progenitor cell) that express the fusion proteins and specifically bind to antigens that are expressed by or are otherwise associated with a disease or condition, such as, for example, a cancer. In certain aspects, a host cell expressing a fusion protein of the instant disclosure has improved (e.g., increased and/or sustained) cell signaling (e.g., cytokine production and/or release (for example, IFN-γ, TNFα, IL-2), phosphorylation of one or more protein associated with an immune cell response to antigen-binding, or the like, such as phosphorylation of LAT, PLC-γ1, SLP-76, or any combination thereof) and/or activity (e.g., mobilization of intracellular calcium, killing activity, secretion of a cytokine, proliferation, earlier activation following stimulation, or the like) in response to antigen relative to a host cell expressing a reference fusion protein that does not comprise in an intracellular component a sequence or motif from a CD3ε, CD3γ, and/or CD3δ protein, as disclosed herein. In some embodiments, a host cell expressing a fusion protein of this disclosure has improved (e.g., earlier and/or stronger, relative to a host cell expressing a reference fusion protein) cell signaling and/or activity upon binding to a target antigen that is expressed at a low level or an intermediate level on a target cell (e.g., solid tumor cell) surface, and/or in certain embodiments, upon binding to a target antigen that is expressed at a high level on a target cell surface.

In certain embodiments, a host cell expressing a fusion protein of the present disclosure is capable, when administered to a subject having a tumor comprising cells that expresses an antigen that is specifically bound or recognized by the fusion protein, of reducing or suppressing growth, area, volume, and/or spread of the tumor, of killing tumor cells, and/or of increasing survival of the subject to a greater degree and/or for a longer period of time as compared to a reference subject administered a cell therapy wherein the cells do not comprise a fusion protein as provided herein (e.g., comprise a reference fusion protein that has a similar structure to the fusion protein of the present disclosure but does not comprise a sequence or motif from a CD3ε, CD3γ, and/or CD3δ protein, as disclosed herein).

In certain embodiments, a host cell expressing a fusion protein of the present disclosure is capable of greater antitumor activity as compared to a reference host cell expressing a fusion protein that does not comprise a sequence or motif from a CD3ε, CD3γ, and/or CD3δ protein, as disclosed herein; e.g., host cells expressing a fusion protein of the present disclosure may infiltrate a tumor in a number that is statistically not significantly different as compared to the number of reference host cells infiltrating a reference tumor, but the host cell expressing a fusion protein of the present disclosure has improved antitumor activity (e.g., causes slower growth or stops growth of the tumor, or reduces tumor size, or spread, or the like) versus the reference host cell.

In certain embodiments, a fusion protein of the present disclosure can, following stimulation with antigen, promote (i.e., directly or indirectly) phosphorylation of LAT, SLP-76, and/or PLC-γ1 more efficiently than a reference fusion protein that does not comprise a sequence or motif from a CD3ε, CD3γ, and/or CD3δ protein, as disclosed herein. By way of illustration, in certain embodiments, a fusion protein of the present disclosure is expressed in a host cell (e.g., an immune cell such as, for example, a T cell) and comprises (i) a sequence or motif from a CD3ε, CD3γ, and/or CD3δ protein and (ii) a CD3ζ ITAM or endodomain, and binding of the fusion protein to antigen results in decreased phosphorylation of the CD3ζ ITAM or endodomain as compared to binding of a reference fusion protein comprising a CD3ζ ITAM or endodomain to antigen, but results in a similar or increased phosphorylation of a downstream cell signaling protein, such as, for example, LAT, PLC-γ1, or both.

In certain embodiments, a host cell (e.g., an immune cell such as a T cell) expressing a fusion protein of the present disclosure has improved sensitivity to antigen as compared to a host cell expressing a reference fusion protein that does not contain a sequence or motif from a CD3ε, CD3γ, and/or CD3δ protein, but does not produce more, or does not produce substantially more, of a pro-inflammatory cytokine (e.g., IFN-γ, IL-2, TNF-α) as compared to the host cell expressing the reference fusion protein. Without wishing to be bound by theory, marked increases in production of pro-inflammatory cytokines may cause toxicities. Accordingly, presently disclosed fusion proteins and host cells expressing the same have, in certain embodiments, improved sensitivity for antigen (i.e., at a low, intermediate, and/or high level of antigen expression) but do not increase, or do not substantially increase, a risk of an inflammatory cytokine-associated toxicity.

In certain embodiments, a fusion protein according to the present disclosure comprises a sequence or motif from a CD3ε, CD3γ, and/or CD3δ protein and further comprises a 4-1BB costimulatory domain. In certain embodiments, a reference fusion protein comprises a 4-1BB costimulatory domain and/or a CD28 costimulatory domain.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains or motifs, wherein the domains or motifs are not naturally found together (e.g., in the specified arrangement, order, or number, or at all) in a protein. In certain embodiments, a fusion protein comprises at least two distinct domains or motifs that are not naturally found together in a single peptide or polypeptide. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein is anchored to the cell membrane (e.g., via a transmembrane domain) and comprises an extracellular portion or component (e.g., containing a binding domain and, in certain embodiments, a linker, a spacer, or both) and an intracellular portion or component (e.g., containing a sequence or motif from a CD3 protein as provided herein).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed; or one or more amino acids are mutated, inserted, or deleted), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Kₐ) or a dissociation (K_{D}) constant) or avidity; or an assay measuring phosphorylation or activation of, or by, an immune cell protein such as, for example, Lck, ZAP70, Fyn, PLCγ-1, SLP-76, LAT, or the like, including the assays described herein. The ability of a polypeptide or encoded polypeptide of this disclosure (or a functional variant of the same) to initiate, continue, participate in, propagate, or amplify a cell signaling event or events (e.g., T cell signaling in response to antigen-binding) may be determined by examining the activity, structure, chemical state (e.g., phosphorylation), or interactions of or between the variant polypeptide and an immune cell protein that directly acts (e.g., binds to) therewith, or by examining the activity, localization, structure, expression, secretion, chemical state (e.g., phosphorylation), or interactions of or between other biomolecules known or thought to participate in or be affected by the cell signaling event or events. The ability of a polypeptide or encoded polypeptide of this disclosure (or a functional variant of the same) to initiate, continue, participate in, propagate, or amplify a cell signaling event or events may also be determined by using functional assays of host cell activity, including those described herein for measuring the ability of a host cell to release cytokines, proliferate, selectively kill target cells, or treat a subject having a disease or condition expressing or otherwise associated with an antigen bound by a fusion protein of this disclosure.

As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release). In certain embodiments, a functional portion refers to a “signaling portion” of an effector molecule, effector domain, costimulatory molecule, or costimulatory domain.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). It will be appreciated that in the case of a host cell that comprises a heterologous polynucleotide, the polynucleotide is “heterologous” to progeny of the host cell, whether or not the progeny were themselves manipulated (e.g., transduced) to contain the polynucleotide.

The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“T cell receptor” (TCR) or “TCR complex” refers to a multi-protein complex known as an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins (i.e., antibodies), the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also, like immunoglobulins, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, mouse, rat, rabbit or other mammal.

In certain embodiments, a TCR is found on the surface of T cells (or T lymphocytes) and associates with the CD3 complex. In certain embodiments, a TCR complex comprises a TCR or a functional portion thereof, a dimer comprising two CD3ζ chains, or functional portions or variants thereof, a dimer comprising a CD3δ chain and a CDε chain, or functional portions or variants thereof, and a dimer comprising a CD3γ chain and a CDε chain, or functional portions or variants thereof, any one or more of which may be endogenous or heterologous to the T cell.

As used herein, a “TCR extracellular domain” refers to a portion of a TCR that comprises a TCR ectodomain or a portion thereof, such as a constant domain from a TCRα chain or a TCRβ chain, or an ectodomain from CD3ε, CD3δ, or CD3γ; optionally comprising a TCR variable domain (e.g., a V_{α} and/or a V_{β} domain). In certain embodiments, a TCR extracellular domain (i) comprises or consists of a TCR ectodomain, or a portion thereof, (ii) does not comprise a TCR variable domain (e.g., does not comprise a V_{α} and/or a V_{β}), or (iii) comprises or consists of a TCR constant domain or ectodomain, or a portion thereof, and does not comprise a TCR variable domain. In certain embodiments, a fusion protein of the instant disclosure does not comprise a TCR ectodomain.

“CD3” is a multi-protein complex of six chains (see, Abbas and Lichtman, 2003; Janeway et al., p. 172 and 178, 1999). In mammals, the complex generally comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of T cell receptor chains. The intracellular tails of the CD3γ, CD3δ, and CD3ε chains each contain a single conserved motif known as an immunoreceptor tyrosine-based activation motif or ITAM, whereas each CD3ζ chain has three ITAMs (also referred to as CD3ζ “(a)”, “(b)”, and “(c)” ITAMs herein). In general, ITAMs comprise an amino acid sequence motif “YxxL/I” (SEQ ID NO:1), wherein xx may be any two (i.e., the same or different) amino acids. In certain embodiments, an ITAM comprises a motif YxxL/Ix₍₆₋₈₎YxxL/I (SEQ ID NO:2), wherein x may be any amino acid (i.e., a same or different amino acids over the length of the ITAM). In certain embodiments, an ITAM motif can comprise an amino acid sequence motif according to SEQ ID NO:3, SEQ ID NO:4, or both. Exemplary ITAMs of the present disclosure include those that comprise an amino acid sequence as shown in any one of SEQ ID NOs:6, 12, 15, and 18-20. Other ITAMs from human proteins include those found in B cell receptor-associated proteins (e.g., Igα, Igβ), Fc receptor proteins (e.g., FcRγI, FcRγ2a, FcRγ2b1, FcRγ2a1, FcRγ2b2, FcRγ3a, FcRγ3b, FcRβ1, FcεR), Natural Killer cell receptor proteins (e.g., DAP12), CD5, CD16a, CD16b, CD22, CD23, CD32, CD64, CD79a, CD79b, CD89, CD278, and CD66d. Exemplary amino acid sequences of these ITAM sequences and those from viruses (e.g., BLV gp30; EBV LMP2A) are described in Paul, Fundamental Immunology 307 (Wolters Kluwer; Lippincott; Wilkins & Wilkins; Seventh Ed., 2008). These ITAMs and functional fragments and variants thereof are also contemplated for use in the presently disclosed fusion proteins and host cells, and are hereby incorporated by reference.

CD3, as well as the protein subunits, domains, and sequences therefrom, may be from various animal species, including human, mouse, rat, or other mammals.

In addition to ITAMs, CD3ε and CD3ζ possess additional sequence features that may be involved in or required for T cell signaling; e.g., following recognition of antigen:MHC by a TCR. Human CD3ε includes a proline-rich sequence (PRS) that is N-terminal to the ITAM in the CD3ε cytoplasmic domain. An exemplary PRS amino acid sequence is provided in SEQ ID NO:7. An exemplary fragment of a CD3ε cytoplasmic domain (also referred to as an endodomain) comprising a PRS and an ITAM is provided in SEQ ID NO:8. CD3ε and CDζ both contain at least one Basic Residue Sequence (BRS). In the case of human CD3ε, a BRS is generally located near the junction of the transmembrane and cytoplasmic domains. An exemplary BRS from CD3ε is provided in SEQ ID NO:9. An exemplary sequence of a human CD3ε cytoplasmic domain containing a BRS, a PRS, and an ITAM is provided in SEQ ID SEQ ID NO:111. In certain embodiments, an effector domain or a functional portion or variant thereof from a CD3ε does not comprise a full-length CD3ε and does not comprise an endodomain from CD3ε. In the case of CD3ζ, two BRS are generally located between ITAMs (a) and (b), and a third BRS is located between ITAMs (b) and (c). Exemplary BRS from CD3ζ are provided in SEQ ID NOs:21-23. An exemplary sequence of a human CD3ζ cytoplasmic domain containing ITAMS and BRS is provided in SEQ ID NO:17. In any of the embodiments described herein, a fusion protein of the instant disclosure can comprise an amino acid sequence having least 75% (e.g., at least 75%, 80%, 85%, 90%, 91%, 92%, 93%, 94%, 95%, 96%, 97%, 98%, 99% identity, or more), to a reference amino acid sequence as set forth herein.

In certain embodiments, a fusion protein of the present disclosure, when expressed by a T cell, does not associate with or form a TCR complex (i.e., does not form or associate with a TCR complex comprising a TCR, a dimer comprising two CD3ζ chains, a dimer comprising a CD3δ chain and a CDε chain, and a dimer comprising a CD3γ chain and a CDε chain, any one or more of which components may be endogenous or heterologous to the T cell). Whether two or more proteins form a complex can be determined by any suitable technique, such as, for example, imaging a T cell that expresses a detectably labeled (e.g., fluorescently labeled or bound by a labeled antibody) fusion protein of this disclosure and a fluorescently labeled TCR, co-precipitating a fusion protein and a TCR, or the like.

In certain embodiments, a fusion protein does not comprise an ectodomain and/or a transmembrane domain from CD3δ, CDε, or CD3γ, or any combination thereof. In particular embodiments, a fusion protein does not comprise an ectodomain or a transmembrane domain, or a portion thereof, from CD3δ. In some embodiments, a fusion protein does not comprise an ectodomain or a transmembrane domain, or a portion thereof, from CD3ε. In certain embodiments, a fusion protein does not comprise an ectodomain or a transmembrane domain, or a portion thereof, from CD3γ.

“Major histocompatibility complex molecules” (MHC molecules) refer to glycoproteins that deliver peptide antigens to a cell surface. MHC class I molecules are heterodimers consisting of a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB PO₁₇₃₀). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1).

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein of the present disclosure engineered to contain two or more naturally occurring (or engineered) amino acid sequences linked together in a way that does not occur naturally or does not occur naturally in a host cell, which fusion protein can function as a receptor when present on a surface of a cell. CARs of the present disclosure include an extracellular portion comprising an antigen-binding domain (e.g., obtained or derived from an immunoglobulin or immunoglobulin-like molecule, such as a scFv or scTCR derived from an antibody or TCR specific for a cancer antigen, or an antigen-binding domain derived or obtained from a killer immunoreceptor from an NK cell, or from another protein (natural, recombinant, or synthetic) that has, or is engineered to possess, the ability to specifically bind to an antigen) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)). In certain embodiments, a binding protein comprises a CAR comprising an antigen-specific TCR binding domain (see, e.g., Walseng et al., Scientific Reports 7:10713, 2017; the TCR CAR constructs and methods of which are hereby incorporated by reference in their entirety).

The term “variable region” or “variable domain” refers to the domain of a TCR α-chain or β-chain (or γ-chain and δ-chain for γδ TCRs), or of an antibody heavy or light chain, that is involved in binding to antigen (i.e., contains amino acids and/or other structures that contact antigen and result in binding). The variable domains of the α-chain and β-chain (Vα and Vβ, respectively) of a native TCR generally have similar structures, with each domain comprising four generally conserved framework regions (FRs) and three CDRs. Variable domains of antibody heavy (V_{H}) and light (V_{L}) chains each also generally comprise four generally conserved framework regions (FRs) and three CDRs. In both TCRs and antibodies, framework regions separate CDRs and CDRs are situated between framework regions (i.e., in primary structure).

The terms “complementarity determining region,” and “CDR,” are synonymous with “hypervariable region” or “HVR,” and are known in the art to refer to sequences of amino acids within TCR or antibody variable regions, which, in general, confer antigen specificity and/or binding affinity and are separated from one another in primary structure by framework sequence. In some cases, framework amino acids can also contribute to binding, e.g., may also contact the antigen or antigen-containing molecule. In general, there are three CDRs in each variable region (i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. CDR1 and CDR2 mainly interact with the MHC. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof.

A “therapeutically effective amount” or “effective amount” of a fusion protein or host cell expressing a fusion protein of this disclosure, refers to an amount of fusion proteins or host cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different fusion proteins (e.g., CARs) that specifically bind a an antigen, or a fusion protein of the present disclosure.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### Fusion Proteins

In certain aspects, the present disclosure provides fusion proteins, comprising: (a) an extracellular component comprising a binding domain that specifically binds to an antigen; (b) a transmembrane domain; and (c) an intracellular component comprising an effector domain or a functional portion thereof, wherein the effector domain or functional portion thereof comprises: (i) an Intracellular Tyrosine-based Activation Motif (ITAM) from CD3ε, or a functional variant thereof; (ii) an ITAM from CD3γ, or a functional variant thereof; (iii) an ITAM from CD3δ, or a functional variant thereof, (iv) a Proline Rich Sequence (PRS) from CD3ε, or a functional variant thereof, (v) a Basic Residue Sequence (BRS) from CD3ε and/or CD3ζ, or a functional variant thereof; or (vi) any combination of (i)-(v), wherein: (1) the extracellular domain does not comprise a TCR extracellular domain, preferably does not comprise a TCR ectodomain; and/or (2) the fusion protein does not associate with or form a TCR complex when expressed by a T cell.

In certain embodiments, a fusion protein of the present disclosure, when expressed by a T cell or a cell that otherwise expresses TCR complex proteins, does not associate with or form a TCR complex (i.e., does not form or associate with a TCR complex comprising a TCR, a dimer comprising two CD3ζ chains, a dimer comprising a CD3δ chain and a CDε chain, and a dimer comprising a CD3γ chain and a CDε chain, any one or more of which components may be endogenous or heterologous to the T cell). Whether two or more proteins form a complex can be determined by any suitable technique, such as, for example, imaging a T cell that expresses a detectably labeled (e.g., fluorescently labeled or bound by a labeled antibody) fusion protein of this disclosure and a fluorescently labeled TCR, co-precipitating a fusion protein and a TCR, or the like.

In certain embodiments, a fusion protein of the present disclosure does not comprise an ectodomain and/or a transmembrane domain from CDε, from CD3γ, from CD3δ, or any combination thereof. In some embodiments, a fusion protein does not comprise an ectodomain or a transmembrane domain, or a portion thereof, from CD3ε.

In certain embodiments, a fusion protein does not comprise an ectodomain or a transmembrane domain, or a portion thereof, from CD3δ. In certain embodiments, a fusion protein does not comprise an ectodomain or a transmembrane domain, or a portion thereof, from CD3γ.

In certain aspects, a fusion protein of the present disclosure comprises: (a) an extracellular component comprising a binding domain that specifically binds to an antigen; (b) a transmembrane domain; and (c) an intracellular component comprising an effector domain or a functional portion thereof, wherein the effector domain or functional portion thereof comprises: (i) an Intracellular Tyrosine-based Activation Motif (ITAM) from CD3ε, or a functional variant thereof; (ii) an ITAM from CD3γ, or a functional variant thereof; (iii) an ITAM from CD3δ, or a functional variant thereof, (iv) a Proline Rich Sequence (PRS) from CD3ε, or a functional variant thereof, (v) a Basic Residue Sequence (BRS) from CD3ε and/or CD3ζ, or a functional variant thereof; or (vi) any combination of (i)-(v), and does not comprise an ectodomain or a transmembrane domain, or a portion thereof, from CD3ε, CD3δ, and/or CD3γ.

In any of the presently disclosed embodiments, the effector domain or functional portion thereof comprises an ITAM from CD3ε, or a functional variant thereof, and a PRS from CD3ε, or a functional variant thereof.

In certain embodiments, the fusion protein does not comprise a BRS from CD3ε, or a functional variant thereof.

In certain embodiments, the effector domain or functional fragment thereof comprises an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:8. In certain embodiments, the effector domain or functional fragment thereof comprises one, two, or three, four, or more of the amino acids of SEQ ID NO:5 or SEQ ID NO:111 that are immediately amino-terminal to and/or comprises one, two, or three, four, or more of the amino acids of SEQ ID NO:5 or SEQ ID NO:111 that are immediately carboxy-terminal to the amino acid sequence of SEQ ID NO:8.

In certain embodiments, the effector domain comprises a BRS from CD3ε, or a functional variant thereof.

In certain embodiments, the effector domain comprises an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:5 or 111.

In certain embodiments, the effector domain comprises: an ITAM from CD3γ, or a functional variant thereof; and/or comprises an ITAM from CD3δ, or a functional variant thereof. In certain embodiments, the effector domain comprises: (i) an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:11, optionally wherein one or more of the amino acids corresponding to positions 16 and 17 of SEQ ID NO:11 is not a leucine, and is preferably an alanine or a glycine; and/or (ii) an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:14, optionally wherein one or more of the amino acids corresponding to positions 16 and 17 of SEQ ID NO:14 is not a leucine, and is preferably an alanine or a glycine. In certain embodiments, the effector domain comprises a BRS from CD3ε, or a functional portion or variant thereof.

In certain embodiments, the effector domain or functional fragment thereof comprises the amino acid sequence set forth in SEQ ID NO:12 and further comprises one, two, or three, four, five, six, seven, eight, nine, ten, or more of the amino acids of SEQ ID NO:10 or 11 that are immediately amino-terminal to and/or comprises one, two, or three, four, five, six, seven, or eight of the amino acids of SEQ ID NO:10 or 11 that are immediately carboxy-terminal to the amino acid sequence of SEQ ID NO:12.

In certain embodiments, the effector domain or functional fragment thereof comprises the amino acid sequence set forth in SEQ ID NO:15 and further comprises one, two, three, four, five, six, seven, eight, nine, ten, or more of the amino acids of SEQ ID NO:14 that are immediately amino-terminal to and/or comprises one, two, or three, four, five, six, seven, or eight of the amino acids of SEQ ID NO:14 that are immediately carboxy-terminal to the amino acid sequence of SEQ ID NO:15.

In certain embodiments, the effector domain further comprises an ITAM from CD3ζ, or a functional portion or variant thereof. In further embodiments, the effector domain further comprises two or three of ITAMs (a), (b), and (c) from CD3ζ, or functional portions or variants thereof. In certain embodiments, the effector domain comprises SEQ ID NO:18, SEQ ID NO:19, and/or SEQ ID NO:20, and further comprises one, two, three, four, five, six, seven, eight, nine, ten, or more of the amino acids of SEQ ID NO:17 that are immediately amino-terminal to the amino acid sequence of SEQ ID NO:18, 19, or 20, respectively, and/or comprises one, two, or three, four, five, six, seven, or eight of the amino acids of SEQ ID NO:17 that are immediately carboxy-terminal to the amino acid sequence of SEQ ID NO:18, 19, or 20, respectively.

In certain embodiments, the effector domain comprises an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:17.

In any of the presently disclosed embodiments, a fusion protein can comprise an ITAM motif, a Lck-binding motif, a PRS, a BRS, or sequence comprising the same, having or comprising the amino acid sequence as set forth in any one of SEQ ID NOS:1-9, 11, 12, 14, 15, 18-24, 27-32, or 111, or a functional variant or fragment thereof, or any combination thereof.

A “binding domain” (also referred to as a “binding region” or “binding moiety”), as used herein, refers to a molecule or portion thereof (e.g., peptide, oligopeptide, polypeptide, protein (e.g., a fusion protein)) that possesses the ability to specifically and non-covalently associate, unite, or combine with a target. A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv, Fab, TCR variable regions), receptor ectodomains, ligands (e.g., cytokines, chemokines), or synthetic polypeptides selected for their specific ability to bind to a biological molecule, a molecular complex or other target of interest. In certain embodiments, the binding domain is a scFv, scTCR, or ligand. In certain embodiments, the binding domain is chimeric, human, or humanized.

In certain embodiments, the binding domain is a scFv comprising a V_{H} domain, a V_{L} domain, and a peptide linker. In particular embodiments, a scFv comprises a V_{H} domain joined to a V_{L} domain by a peptide linker, which can be in a V_{H}-linker-V_{L} orientation or in a V_{L}-linker-V_{H} orientation.

Any scFv of the present disclosure may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C). It will be appreciated that any scTCR of the present disclosure may be engineered so that the C-terminal end of the V_{α} domain is linked by a short peptide sequence to the N-terminal end of the V_{β} domain, or vice versa (i.e., (N)V_{α}(C)-linker-(N)V_{β}(C) or (N)V_{β} (C)-linker-(N)V_{α}(C)).

As used herein, “specifically binds” or “specific for” refers to an association or union of a binding protein (e.g., a T cell receptor or a chimeric antigen receptor) or a binding domain (or fusion protein comprising a binding domain) to a target molecule with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Binding proteins or binding domains (or fusion proteins thereof) may be classified as “high-affinity” binding proteins or binding domains (or fusion proteins thereof) or as “low-affinity” binding proteins or binding domains (or fusion proteins thereof). “High-affinity” binding proteins or binding domains refer to those binding proteins or binding domains having a Kₐ of at least 10⁷ M⁻¹, at least 10⁸ M⁻¹, at least 10⁹ M¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵ M⁻¹. Alternatively, affinity may be defined as an equilibrium dissociation constant (K_{D}) of a particular binding interaction with units of M (e.g., 10⁻⁵ M to 10⁻¹³ M).

In further embodiments, a fusion protein of the instant disclosure specifically binds to a target antigen (e.g., a cancer antigen such as, for example, a CD19, CD20, CD22, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, SSX-2, PRAME, HA-1, PSA, ephrin A2, ephrin B2, an NKG2D, NY-ESO-1, TAG-72, mesothelin, NY-ESO, 5T4, BCMA, FAP, Carbonic anhydrase 9, ERBB2, BRAF^{V600E}, CD79a, CD79b, SLAMF7, or CEA antigen; an autoimmune antigen; or an antigen associated with an infection).

Sources of binding domains specific for these antigens are known in the art. Exemplary binding domains specific for ROR1 and CD19 antigens, including CDRs thereof, are disclosed SEQ ID NOs:55-102. A fusion protein of the present disclosure can, in certain embodiments, comprise a variable domain and/or one or more CDRs (e.g., three heavy chain CDRs and three light chain CDRs) according to any one of these exemplary binding domain sequences (e.g., from an anti-ROR1 or anti-CD19 scFv, or R11 antibody, R12 antibody, Y31 antibody, 2A2 antibody, or FMC63 antibody), or can comprise a functional variant sequence thereof.

In certain embodiments, a fusion protein of the instant disclosure is expressed by a host cell (e.g, an immune system cell such as, for example, a T cell) and the host cell specifically recognizes (i.e., comprising specific binding) and initiates an immune response to a target cell expressing a reduced (e.g., as compared to a reference baseline level of expression of the same or a different antigen (e.g., as compared to an average level among subjects or tumors having a same disease or disease state), or to a prior level of expression by a disease site, such as a tumor, in the subject) or low or intermediate level of the antigen). In certain embodiments, a reduced level of expression comprises at a reduction of at least about 5%, 10%, 20%, 30%, 40%, 50%, 60%, 70%, 80%, 90%, 91%, 92%, 93%, 94%, 95%, 96%, 97%, 98%, 99%, or more, as compared to a reference baseline or previous subject level. In certain embodiments, a reduced level of expression is a reduction of 1, 5, 10, 20, 30, 40, 50, 60, 70, 80, 90, or 100-fold, or more, as compared to a prior or reference level of expression. An expression level of an antigen can be determined using any art-accepted methodology, including, for example, use of labeled antibodies, Western Blot, RNA-Seq, or the like. In certain embodiments, a low antigen density comprises less than about 10,000, less than about 9,000, less than about 8,000, less than about 7,000, less than about 6,000, less than about 5,000, less than about 4,000, less than about 3,000, less than about 2,000, less than about 1,000, less than about 500, less than about 200, less than about 190, less than about 180, less than about 170, less than about 160, less than about 150, less than about 140, less than about 130, less than about 120, less than about 110, about 100, less than about 90, less than about 80, less than about 70, less than about 60, less than about 50, less than about 40, less than about 30, less than about 20, or less than about 10 molecules of the antigen expressed on the surface of a target cell. In some embodiments, an intermediate antigen density comprises about 10,000 to about 20,000 molecules of antigen expressed on the surface of a target cell. In some embodiments, a high antigen density comprises about 20,000 molecules or more of antigen expressed on the surface of a target cell.

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for a fusion protein is measured by assessing binding to various concentrations of tetramers, for example, by flow cytometry using labeled tetramers. In some examples, apparent K_{D} of a fusion protein is measured using 2-fold dilutions of labeled tetramers at a range of concentrations, followed by determination of binding curves by non-linear regression, apparent K_{D} being determined as the concentration of ligand that yielded half-maximal binding.

As used herein, an “effector domain” is an intracellular portion or domain of a fusion protein or receptor that can directly or indirectly promote a biological or physiological response (e.g., an immune response) in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

An effector domain may directly promote a cellular response (e.g., immune response) when it contains one or more signaling domains or motifs, such as an Intracellular Tyrosine-based Activation Motif (ITAM), such as those found in costimulatory molecules. Without wishing to be bound by theory, it is believed that ITAMs are important for T cell activation following ligand (e.g., antigen) engagement by a T cell receptor or by a fusion protein comprising a T cell effector domain. In certain embodiments, the intracellular component or functional portion thereof comprises an ITAM. Exemplary effector domains include those from CD3ε, CD3δ, CD3ζ, CD25, CD79A, CD79B, CARD 11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In certain embodiments, an effector domain comprises a lymphocyte receptor signaling domain (e.g., CD3ζ or a functional portion or variant thereof).

In certain embodiments, the intracellular component of the fusion protein further comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both. Exemplary amino acid sequences of certain costimulatory domains are provided in SEQ ID NOs: and 25 and 47-49. In certain embodiments, a costimulatory domain or functional portion or variant thereof comprises or consists of an amino acid sequence having at least 75% (i.e., at least 75%, 80%, 85%, 90%, 91%, 92%, 93%, 94%, 95%, 96%, 97%, 98%, 99%, or more) identity to any one of SEQ ID NOs:25 and 47-49.

In certain embodiments, an effector domain comprises a CD3ζ endodomain or a functional (e.g., signaling) portion thereof, or a functional variant thereof. In certain embodiments, an effector domain comprises a CD27 endodomain or a functional (e.g., signaling) portion thereof, or a functional variant thereof. In certain embodiments, an effector domain comprises a CD28 endodomain or a functional (e.g., signaling) portion thereof, or a functional variant thereof. In certain embodiments, an effector domain comprises a 4-1BB endodomain or a functional (e.g., signaling) portion thereof, or a functional variant thereof. In certain embodiments, an effector domain comprises an OX40 endodomain or a functional (e.g., signaling) portion thereof, or a functional variant thereof. In certain embodiments, an effector domain comprises a CD2 endodomain or a functional (e.g., signaling) portion thereof, or a functional variant thereof. In certain embodiments, an effector domain comprises a CD5 endodomain or a functional (e.g., signaling) portion thereof, or a functional variant thereof. In certain embodiments, an effector domain comprises an ICAM-1 endodomain or a functional (e.g., signaling) portion thereof, or a functional variant thereof. In certain embodiments, an effector domain comprises a LFA-1 endodomain or a functional (e.g., signaling) portion thereof, or a functional variant thereof. In certain embodiments, an effector domain comprises an ICOS endodomain or a functional (e.g., signaling) portion thereof, or a functional variant thereof.

In certain embodiments, the intracellular domain of a fusion protein according to the present disclosure comprises a costimulatory domain, an endodomain or effector domain from CD3ζ or a functional portion or variant thereof, and an effector domain from CD3ε or a functional portion or variant thereof, wherein the effector domain from CD3ε or functional portion or variant thereof optionally comprises or consists essentially of a PRS and an ITAM from CD3ε.

In further embodiments, the effector domain from CD3ε or functional portion or variant thereof is disposed between the costimulatory domain or functional portion or variant thereof and the endodomain or effector domain from CD3ζ or functional portion or variant thereof. In certain embodiments, the costimulatory domain or functional portion or variant thereof is from 4-1BB. In certain embodiments, the intracellular component of the fusion protein comprises, in amino-terminal to carboxy-terminal direction: (i) the costimulatory domain or functional portion or variant thereof; (ii) the effector domain from CD3ε or functional portion or variant thereof; and (iii) the endodomain or effector domain from CD3ζ or functional portion or variant thereof.

In other embodiments, the endodomain or effector domain from CD3ζ or functional portion or variant thereof is disposed between the costimulatory domain and the effector domain from CD3ε or functional portion or variant thereof. In certain embodiments, the costimulatory domain is from 4-1BB. In certain embodiments, the intracellular component of the fusion protein comprises, in amino-terminal to carboxy-terminal direction: (i) the costimulatory domain or functional portion or variant thereof; (ii) the endodomain or effector domain from CD3ζ or functional portion or variant thereof; and (iii) the effector domain from CD3ε or functional portion or variant thereof.

An extracellular component and an intracellular component of the present disclosure are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about amino acids to about 30 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof). An exemplary CD28 transmembrane domain amino acid sequence is provided in SEQ ID NO:26. In certain embodiments, a transmembrane domain comprises or consists of an amino acid sequence having at least 75%, 80%, 85%, 90%, 91%, 92%, 93%, 94%, 95%, 96%, 97%, 98%, 99%, or more identity to SEQ ID NO:26.

In certain embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the fusion protein to enable proper contact between the host cell and a target cell, antigen binding, and activation (Patel et al., Gene Therapy 6: 412-419, 1999). Linker length may be varied to maximize antigen recognition based on the selected target molecule, selected binding epitope, or antigen binding domain size and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser). In some embodiments, the extracellular domain comprises a glycine-serine linker that is not comprised in the binding domain; e.g., is disposed between the transmembrane domain and the binding domain. For example, in certain embodiments, a fusion protein comprises an extracellular domain comprising a first glycine-serine linker disposed between the transmembrane domain and the binding domain, and the binding domain may comprise a scFv or an scTCR that comprises a second glycine-serine linker, wherein the first and second glycine-serine linkers may be a same or a different glycine-serine linker and may be of a same or a different length.

Exemplary linker amino acid sequences of the present disclosure are provided in SEQ ID NOs:103-110.

Linkers of the present disclosure also include immunoglobulin constant regions (i.e., CH1, CH2, CH3, or CL, of any isotype) and portions and variants thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or a portion thereof. In certain embodiments, the linker comprises an extracellular domain from CD4, or a portion thereof. In some embodiments, the linker comprises an extracellular domain from CD8, or a portion thereof.

In any of the embodiments described herein, a linker may comprise a hinge region or a portion thereof. An exemplary hinge sequence of the present disclosure is provided in SEQ ID NO:33. Hinge regions are flexible amino acid polymers of variable length and sequence (typically rich in proline and cysteine amino acids) and connect typically larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker as described herein.

In certain embodiments, one or more of the extracellular component, the binding domain, the linker, the transmembrane domain, the intracellular component, the effector domain, or the costimulatory domain further comprise one or more junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a fusion protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In certain embodiments, a protein tag of a fusion protein of this disclosure comprises a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, Strep tags (e.g., Strep-Tag; Strep-Tag II; and variants thereof, including those disclosed in, for example, Schmidt and Skerra, Nature Protocols, 2:1528-535 (2007), U.S. Pat. No. 7,981,632; and PCT Publication No. WO 2015/067768, the strep-tag peptides, step-tag-peptide-containing polypeptides, and sequences of the same, are incorporated herein by reference), or any combination thereof.

In any of the embodiments described herein, a fusion protein can be or can comprise a CAR or a TCR. Methods for making fusion proteins, including CARs, are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Brentjens et al., 2007, Clin. Cancer Res. 13:5426, and Walseng et al., Scientific Reports 7:10713, 2017, the techniques of which are herein incorporated by reference.

Methods for producing engineered TCRs are described in, for example, Bowerman et al., Mol. Immunol., 46(15):3000 (2009), the techniques of which are herein incorporated by reference. In certain embodiments, the antigen-binding fragment of the TCR comprises a single chain TCR (scTCR), which comprises both the TCR Vα and Vβ domains TCR, but only a single TCR constant domain (Cα or Cβ). In certain embodiments, the antigen-binding fragment of the TCR, or chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

Methods useful for isolating and purifying recombinantly produced soluble fusion proteins, by way of example, may include obtaining supernatants from suitable host cell/vector systems that secrete the recombinant soluble fusion protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble fusion protein described herein include batch cell culture, which is monitored and controlled to maintain appropriate culture conditions. Purification of the soluble fusion protein may be performed according to methods described herein and known in the art and that comport with laws and guidelines of domestic and foreign regulatory agencies.

Fusion proteins as described herein may be functionally characterized according to any of a large number of art-accepted methodologies for assaying host cell (e.g., T cell) activity, including determination of T cell binding, activation or induction and also including determination of T cell responses that are antigen-specific. Examples include determination of intracellular calcium, T cell proliferation, T cell cytokine release, antigen-specific T cell stimulation, MHC-restricted T cell stimulation, CTL activity (e.g., by detecting ⁵¹Cr or Europium release from pre-loaded target cells), changes in T cell phenotypic marker expression, phosphorylation of certain T cell proteins, and other measures of T-cell functions. Procedures for performing these and similar assays are described herein and/or may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, M A (1986); Mishell and Shigii (eds.) Selected Methods in Cellular Immunology, Freeman Publishing, San Francisco, CA (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Assays for determining intracellular calcium are known and include, for example, fluorescence microscopy using the Ca²⁺-sensitive dye Fluo-4 AM (BD Pharmingen™). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-0, and Type 2 cytokines, such as IL-4, IL-5, IL-9, IL-10, and IL-13.

In any of the presently disclosed embodiments, a fusion protein, when expressed by a host cell, which optionally is an immune system cell such as a T cell, provides for or promotes: (i) improved cell signaling, and/or activity in response to antigen relative to a host cell expressing a reference fusion protein, wherein improved cell signaling optionally comprises increased and/or sustained cytokine production and/or release, and/or phosphorylation of one or more protein associated with an immune cell response to antigen-binding, or the like, such as LAT, PLC-γ1, SLP-76, or any combination thereof; (ii) improved cell activity in response to antigen relative to a host cell expressing a reference fusion protein, wherein improved cell signaling optionally comprises increased mobilization of intracellular calcium, killing activity, proliferation, earlier activation in response to antigen, or any combination thereof, (iii) improved cell signaling and/or activity, relative to a host cell expressing a reference fusion protein, upon binding to a target antigen that is expressed at a low level or an intermediate level on a target cell surface; (iv) reducing or suppressing growth, area, volume, and/or spread of a tumor that expresses an antigen that is recognized and/or specifically bound by the fusion protein, of killing tumor cells, and/or of increasing survival of the subject to a greater degree and/or for a longer period of time as compared to a reference subject administered a host cell expressing a reference fusion protein; (iv) more efficient phosphorylation of LAT, SLP-76, and/or PLC-γ1 as compared to a reference fusion protein expressed by a host cell; (v) improved sensitivity to antigen as compared to a host cell expressing a reference fusion protein, but does not produce more, or substantially more, of a pro-inflammatory cytokine as compared to the host cell expressing the reference fusion protein; or (vi) any combination of (i)-(v).

In certain embodiments, the intracellular component of the fusion protein comprises a costimulatory domain, or a functional portion or variant thereof, from 4-1BB. In certain embodiments, the reference protein comprises an intracellular domain comprising a 4-1BB costimulatory domain and/or a CD28 costimulatory domain.

In certain embodiments, the fusion protein and the reference protein each comprise an endodomain or effector domain from CD3ζ or a functional portion or variant thereof. In certain embodiments, the host cell expressing the fusion protein and the host cell expressing the reference fusion protein are each an immune system cell, optionally a T cell, optionally a CD8 T cell, a CD4 T cell, or both.

### Polynucleotides, Vectors, and Host Cells

In certain aspects, nucleic acid molecules are provided that encode any one or more of the fusion proteins as described herein. In certain embodiments, a polynucleotide encoding a fusion protein comprises, or consists of, a polynucleotide having at least 70% (i.e., at least 70%, 75%, 80%, 85%, 90%, 91%, 92%, 93%, 94%, 95%, 96%, 97%, 98%, 99%, or more) identity to the nucleotide sequence set forth in any one of SEQ ID NOs.:34-46. In certain embodiments, a fusion-protein encoding polynucleotide comprises a polynucleotide that encodes a CD3ε intracellular sequence and has at least 70% (i.e., at least 70%, 75%, 80%, 85%, 90%, 91%, 92%, 93%, 94%, 95%, 96%, 97%, 98%, 99%, or more) identity to SEQ ID NO:34. In certain embodiments, a fusion-protein encoding polynucleotide comprises a polynucleotide that encodes a CD3ε_PRS_ITAM sequence and has at least 70% (i.e., at least 70%, 75%, 80%, 85%, 90%, 91%, 92%, 93%, 94%, 95%, 96%, 97%, 98%, 99%, or more) identity to SEQ ID NO:35. In certain embodiments, a fusion-protein encoding polynucleotide comprises a polynucleotide that encodes a CD3ζ intracellular sequence and has at least 70% (i.e., at least 70%, 75%, 80%, 85%, 90%, 91%, 92%, 93%, 94%, 95%, 96%, 97%, 98%, 99%, or more) identity to SEQ ID NO:36. In certain embodiments, a fusion-protein encoding polynucleotide comprises a polynucleotide that encodes a 4-1BB signaling sequence and has at least 70% (i.e., at least 70%, 75%, 80%, 85%, 90%, 91%, 92%, 93%, 94%, 95%, 96%, 97%, 98%, 99%, or more) identity to SEQ ID NO:37. In certain embodiments, a fusion-protein encoding polynucleotide comprises a polynucleotide that encodes a CD28 transmembrane sequence and has at least 70% identity to SEQ ID NO:38. In certain embodiments, a fusion-protein encoding polynucleotide comprises a polynucleotide that encodes a hinge sequence and has at least 70% (i.e., at least 70%, 75%, 80%, 85%, 90%, 91%, 92%, 93%, 94%, 95%, 96%, 97%, 98%, 99%, or more) identity to SEQ ID NO:39. In certain embodiments, a fusion-protein encoding polynucleotide comprises a polynucleotide that encodes a CD28tm_4-1BB signal_CD3ε_PRS_ITAM CD3ζ sequence and has at least 70% (i.e., at least 70%, 75%, 80%, 85%, 90%, 91%, 92%, 93%, 94%, 95%, 96%, 97%, 98%, 99%, or more) identity to SEQ ID NO:40. In certain embodiments, a fusion-protein encoding polynucleotide comprises a polynucleotide that encodes a CD28tm_4-1BB signal_CD3ε_CD3ζ sequence has at least 70% (i.e., at least 70%, 75%, 80%, 85%, 90%, 91%, 92%, 93%, 94%, 95%, 96%, 97%, 98%, 99%, or more) identity to SEQ ID NO:41. In certain embodiments, a fusion-protein encoding polynucleotide comprises a polynucleotide that encodes a CD28tm_4-1BB signal_CD3ζ _CD3ε sequence and has at least 70% (i.e., at least 70%, 75%, 80%, 85%, 90%, 91%, 92%, 93%, 94%, 95%, 96%, 97%, 98%, 99%, or more) identity to SEQ ID NO:42. In certain embodiments, a fusion-protein encoding polynucleotide comprises or consists of a polynucleotide having at least 70% (i.e., at least 70%, 75%, 80%, 85%, 90%, 91%, 92%, 93%, 94%, 95%, 96%, 97%, 98%, 99%, or more) identity to SEQ ID NO:43. In certain embodiments, a fusion-protein encoding polynucleotide comprises or consists of a polynucleotide having at least 70% (i.e., at least 70%, 75%, 80%, 85%, 90%, 91%, 92%, 93%, 94%, 95%, 96%, 97%, 98%, 99%, or more) identity to SEQ ID NO:44. In certain embodiments, a fusion-protein encoding polynucleotide comprises or consists of a polynucleotide having at least 70% (i.e., at least 70%, 75%, 80%, 85%, 90%, 91%, 92%, 93%, 94%, 95%, 96%, 97%, 98%, 99%, or more) identity to SEQ ID NO:45. In certain embodiments, a fusion-protein encoding polynucleotide comprises or consists of a polynucleotide having at least 70% (i.e., at least 70%, 75%, 80%, 85%, 90%, 91%, 92%, 93%, 94%, 95%, 96%, 97%, 98%, 99%, or more) identity to SEQ ID NO:46.

In certain embodiments, a polynucleotide encoding a fusion protein further comprises a polynucleotide encoding a marker. Exemplary markers include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt (e.g., SEQ ID NO:54), CD19t, CD34t, or NGFRt.

In any of the embodiments described herein, a fusion protein-encoding polynucleotide can further comprise a polynucleotide that encodes a marker and a polynucleotide that encodes a self-cleaving polypeptide, wherein the polynucleotide encoding the self-cleaving polypeptide is located between the polynucleotide encoding the fusion protein and the polynucleotide encoding the marker. When the fusion-protein encoding polynucleotide, marker-encoding polynucleotide, and self-cleaving polypeptide are expressed by a host cell, the fusion protein and the marker will be present on the host cell surface as separate molecules. In certain embodiments, a self-cleaving polypeptide comprises a 2A peptide from porcine teschovirus-1 (P2A, Thosea asigna virus (T2A, equine rhinitis A virus (E2A), or foot-and-mouth disease virus (F2A)) (see, e.g., SEQ ID NOs.:50-53). Further exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety).

In any of the embodiments described herein, a self-cleaving polypeptide encoded by a chimeric polynucleotide of this disclosure encodes a P2A, a T2A, an E2A, or a F2A.

In any of the embodiments described herein, a polynucleotide of the present disclosure (e.g., a fusion protein-encoding polynucleotide or polynucleotide encoding a marker) may be codon-optimized for a host cell containing the polynucleotide (see, e.g, Scholten et al., Clin. Immunol. 119:135-145 (2006). Codon optimization can be performed using, e.g., the GenScript® OptimumGene™ tool. Codon-optimized sequences include sequences that are partially codon-optimized (i.e., one or more of the codons is optimized for expression in the host cell) and those that are fully codon-optimized.

A polynucleotide encoding a desired fusion protein of this disclosure can be inserted into an appropriate vector (e.g., viral vector or non-viral plasmid vector) for introduction into a host cell of interest (e.g., an immune cell, such as a T cell).

In further aspects, expression constructs are provided, wherein the expression constructs comprise a polynucleotide of the present disclosure (e.g., a fusion protein-encoding polynucleotide or a polynucleotide encoding a tagged marker) operably linked to an expression control sequence (e.g., a promoter). In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell) for use in adoptive transfer therapy (e.g., targeting a cancer antigen). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to fusion proteins of the present disclosure.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or T_{CM}).

Effector T cells (T_{E}) refers to antigen-experienced CD8⁺ cytotoxic T lymphocytes that have decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Tr1, Th3, CD8⁺CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells.

In certain embodiments, the immune system cell is a CD4+ T cell, a CD8+ T cell, a CD4− CD8− double negative T cell, a γδ T cell, a natural killer cell (e.g., NK cell or NK-T cell), a dendritic cell, a B cell, or any combination thereof. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, a host cell that comprises a heterologous polynucleotide encoding a fusion protein is an immune cell which is modified to reduce or eliminate expression of one or more endogenous genes that encode a polypeptide product selected from PD-1, LAG-3, CTLA4, TIM3, TIGIT, an HLA molecule, a TCR molecule, or any component or combination thereof.

Without wishing to be bound by theory, certain endogenously expressed immune cell proteins may downregulate the immune activity of a modified immune host cell (e.g., PD-1, LAG-3, CTLA4, TIGIT), or may compete with a heterologous fusion protein of the present disclosure for expression by the host cell, or may interfere with the binding activity of a heterologously expressed fusion protein of the present disclosure and interfere with the immune host cell binding to a target cell that expresses an antigen, or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and allows universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013) the gene editing techniques, compositions, and adoptive cell therapies of which are herein incorporated by reference in their entirety). As used herein, the term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene.

In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a host cell (e.g., immune cell) of the present disclosure is engineered so that expression of a presently disclosed fusion protein by the host cell is modulated (e.g., controlled) by binding of the host cell to an antigen that is not the same antigen as the antigen to which the fusion protein specifically binds.

For example, a host cell can comprise (i) a polynucleotide encoding an engineered (i.e., synthetic) Notch receptor comprising (a) an extracellular component comprising a binding domain that binds to an antigen, which is a different antigen than the antigen to which the fusion protein binds, (b) a Notch core domain, or a functional portion or variant thereof; and (c) an intracellular component comprising a transcriptional factor (i.e., a polypeptide capable of activating or increasing, or inhibiting, repressing or reducing, transcription of a target nucleotide sequence (e.g., a gene) or set of target nucleotide sequences); and (ii) the heterologous polynucleotide encoding a fusion protein as disclosed herein and comprising an expression control sequence that can be recognized or bound by the transcriptional factor, wherein binding of the engineered Notch receptor to antigen leads to release of the transcriptional factor from the engineered Notch receptor (e.g., by protease-driven cleavage), which can, in turn, drive transcription of the fusion protein. See, e.g., Morsut et al., Cell 164:780-791 (2016) and PCT Published Application No. WO 2016/138034A1, which synthetic Notch constructs are incorporated herein by reference. Briefly, such “logic-gated” expression systems may be useful to modulate expression of a fusion protein of this disclosure so that the expression occurs only, or preferentially, when the host cell encounters a first antigen (i.e., that can be bound by the synthetic Notch receptor) that is only expressed by, or is principally expressed by, or has a higher expression level on cancer cells as compared to healthy cells. Such embodiments may reduce “on-target off-tissue” recognition by a fusion protein in circumstances where the antigen recognized by the fusion protein is expressed by healthy cells.

In other aspects, kits are provided comprising (a) a vector or an expression construct as described herein and (b) reagents for transducing the vector or the expression construct into a host cell.

### Uses

The present disclosure also provides methods for treating a disease or condition, wherein the methods comprise administering to a subject in need thereof an effective amount of a host cell, composition, or unit dose of the present disclosure, wherein the disease or condition expresses or is otherwise associated with the antigen that is specifically bound by the fusion protein. In certain embodiments, the disease or condition is a hyperproliferative or proliferative disease, such as a cancer, an autoimmune disease, or an infectious disease (e.g., viral, bacterial, fungal, or parasitic).

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In certain embodiments, a cancer treatable according to the presently disclosed methods and uses comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof. In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, the host cell is an allogeneic cell, a syngeneic cell, or an autologous cell.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a fusion protein as described herein is administered intravenously, intraperitoneally, intratumorally, into the bone marrow, into a lymph node, or into the cerebrospinal fluid so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a fusion protein of the present disclosure, or a composition comprising the host cell. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁹ cells, or 10¹⁰ cells or more, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells.

Unit doses are also provided herein which comprise a host cell (e.g., a modified immune cell comprising a polynucleotide of the present disclosure) or host cell composition of this disclosure. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA⁻ CD3⁺ CD8⁺ and engineered CD45RA⁻ CD3⁺CD4⁺ T_{M} cells.

Also contemplated are pharmaceutical compositions that comprise fusion proteins or cells expressing the fusion proteins as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell is a hematopoietic progenitor cell or a human immune cell. In certain embodiments, the immune system cell is a CD4⁺ T cell, a CD8⁺ T cell, a CD4⁻ CD8⁻ double-negative T cell, a γδ T cell, a natural killer cell, a dendritic cell, or any combination thereof. In certain embodiments, the immune system cell is a naïve T cell, a central memory T cell, a stem cell memory T cell, an effector memory T cell, or any combination thereof. In particular embodiments, the cell is a CD4⁺ T cell. In particular embodiments, the cell is a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., host cell, fusion protein, unit dose, or composition) refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a tagged protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

In further aspects, kits are provided that comprise (a) a host cell, (b) a composition, or (c) a unit dose as described herein.

Methods according to this disclosure may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a fusion protein of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, TL-iRA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a fusion protein of the present disclosure (or an engineered host cell expressing the same) with one or more inhibitor of any one of the following immune suppression components, singly or in any combination.

In certain embodiments, a fusion protein is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, a fusion protein of the present disclosure (or an engineered host cell expressing the same) is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a fusion protein of the present disclosure (or an engineered host cell expressing the same) is used in combination with a LAG3 inhibitor, such as LAG525, IMP321, IMP701, 9H12, BMS-986016, or any combination thereof.

In certain embodiments, a fusion protein is used in combination with an inhibitor of CTLA4. In particular embodiments, a fusion protein of the present disclosure (or an engineered host cell expressing the same) is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a fusion protein of the present disclosure (or an engineered host cell expressing the same) is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a fusion protein of the present disclosure (or an engineered host cell expressing the same) is used in combination with an inhibitor of CD244.

In certain embodiments, a fusion protein of the present disclosure (or an engineered host cell expressing the same) is used in combination with an inhibitor of BLTA, HVEM, CD160, or any combination thereof. Anti CD-160 antibodies are described in, for example, PCT Publication No. WO 2010/084158.

In certain embodiments, a fusion protein of the present disclosure (or an engineered host cell expressing the same) is used in combination with an inhibitor of TIM3.

In certain embodiments, a fusion protein of the present disclosure (or an engineered host cell expressing the same) is used in combination with an inhibitor of Gal9.

In certain embodiments, a fusion protein of the present disclosure (or an engineered host cell expressing the same) is used in combination with an inhibitor of adenosine signaling, such as a decoy adenosine receptor.

In certain embodiments, a fusion protein of the present disclosure (or an engineered host cell expressing the same) is used in combination with an inhibitor of A2aR.

In certain embodiments, a fusion protein of the present disclosure (or an engineered host cell expressing the same) is used in combination with an inhibitor of KIR, such as lirilumab (BMS-986015).

In certain embodiments, a fusion protein of the present disclosure (or an engineered host cell expressing the same) is used in combination with an inhibitor of an inhibitory cytokine (typically, a cytokine other than TGFβ) or Treg development or activity.

In certain embodiments, a fusion protein of the present disclosure (or an engineered host cell expressing the same) is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a fusion protein of the present disclosure (or an engineered host cell expressing the same) is used in combination with an arginase inhibitor, such as N(omega)-Nitro-L-arginine methyl ester (L-NAME), N-omega-hydroxy-nor-l-arginine (nor-NOHA), L-NOHA, 2(S)-amino-6-boronohexanoic acid (ABH), S-(2-boronoethyl)-L-cysteine (BEC), or any combination thereof.

In certain embodiments, a fusion protein of the present disclosure (or an engineered host cell expressing the same) is used in combination with an inhibitor of VISTA, such as CA-170 (Curis, Lexington, Mass.).

In certain embodiments, a fusion protein of the present disclosure (or an engineered host cell expressing the same) is used in combination with an inhibitor of TIGIT such as, for example, COM902 (Compugen, Toronto, Ontario Canada), an inhibitor of CD155, such as, for example, COM701 (Compugen), or both.

In certain embodiments, a fusion protein of the present disclosure (or an engineered host cell expressing the same) is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a fusion protein of the present disclosure (or an engineered host cell expressing the same) is used in combination with a LAIR1 inhibitor.

In certain embodiments, a fusion protein of the present disclosure (or an engineered host cell expressing the same) is used in combination with an inhibitor of CEACAM-1, CEACAM-3, CEACAM-5, or any combination thereof.

In certain embodiments, a fusion protein of the present disclosure (or an engineered host cell expressing the same) is used in combination with an agent that increases the activity (i.e., is an agonist) of a stimulatory immune checkpoint molecule. For example, a fusionprotein of the present disclosure (or an engineered host cell expressing the same) can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a fusion protein of the present disclosure (or an engineered host cell expressing the same) with one or more agonist of a stimulatory immune checkpoint molecule, including any of the foregoing, singly or in any combination.

In certain embodiments, a combination therapy comprises a fusion protein of the present disclosure (or an engineered host cell expressing the same) and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a fusion protein and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering a fusion protein of the present disclosure (or an engineered host cell expressing the same) and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates—busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines are used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

The present disclosure also includes the following exemplary embodiments.

Embodiment 1. A fusion protein, comprising:
(a) an extracellular component comprising a binding domain that specifically binds to an antigen;
(b) a transmembrane domain; and
(c) an intracellular component comprising an effector domain or a functional portion thereof, wherein the effector domain or functional portion or variant thereof comprises:
   (i) an Intracellular Tyrosine-based Activation Motif (ITAM) from CD3ε, or a functional portion or variant thereof,
   (ii) an ITAM from CD3γ, or a functional portion or variant thereof,
   (iii) an ITAM from CD3δ, or a functional portion or variant thereof;
   (iv) a Proline Rich Sequence (PRS) from CD3ε, or a functional portion or variant thereof,
   (v) a Basic Residue Sequence (BRS) from CD3ε, or a functional portion or variant thereof, or
   (vi) any combination of (i)-(v),
wherein
   (1) the extracellular domain does not comprise a TCR ectodomain; and/or
   (2) the fusion protein does not associate with or form a TCR complex when expressed by a T cell.

Embodiment 2. A fusion protein, comprising:
(a) an extracellular component comprising a binding domain that specifically binds to an antigen;
(b) a transmembrane domain; and
(c) an intracellular component comprising an effector domain or a functional portion thereof, wherein the effector domain or functional portion thereof comprises:
   (i) an Intracellular Tyrosine-based Activation Motif (ITAM) from CD3ε, or a functional variant thereof;
   (ii) an ITAM from CD3γ, or a functional variant thereof,
   (iii) an ITAM from CD3δ, or a functional variant thereof;
   (iv) a Proline Rich Sequence (PRS) from CD3ε, or a functional variant thereof;
   (v) a Basic Residue Sequence (BRS) from CD3ε and/or CD3ζ, or a functional variant thereof, or
   (vi) any combination of (i)-(v),
   and does not comprise an ectodomain or a transmembrane domain, or a portion thereof, from CD3ε, CD3δ, and/or CD3γ.

Embodiment 3. The fusion protein of embodiment 1 or 2, wherein the effector domain or functional portion thereof comprises an ITAM from CD3ε, or a functional variant thereof, and a PRS from CD3ε, or a functional variant thereof.

Embodiment 4. The fusion protein of any one of embodiments 1-3, wherein the fusion protein does not comprise a BRS from CD3ε, or a functional variant thereof.

Embodiment 5. The fusion protein of any one of embodiments 1-4, wherein the effector domain or functional fragment thereof comprises an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:8.

Embodiment 6. The fusion protein of any one of embodiments 1-3 or 5, wherein the effector domain comprises a BRS from CD3ε, or a functional variant thereof.

Embodiment 7. The fusion protein of any one of embodiments 1-6, wherein the effector domain comprises an amino acid sequence having at least 75% (i.e., at least 75%, 80%, 85%, 90%, 91%, 92%, 93%, 94%, 95%, 96%, 97%, 98%, 99%. or more) identity to the amino acid sequence shown in SEQ ID NO:5 or 111.

Embodiment 8. The fusion protein of any one of embodiments 1-7, wherein the effector domain comprises: an ITAM from CD3γ, or a functional variant thereof; and/or comprises an ITAM from CD3δ, or a functional variant thereof.

Embodiment 9. The fusion protein of embodiment 8, wherein the effector domain comprises:
(i) an amino acid sequence having at least 75% (i.e., at least 75%, 80%, 85%, 90%, 91%, 92%, 93%, 94%, 95%, 96%, 97%, 98%, 99%, or more) identity to the amino acid sequence shown in SEQ ID NO:11, wherein one or more of the amino acids corresponding to positions 16 and 17 of SEQ ID NO:11 is optionally not a leucine, and is preferably an alanine or a glycine; and/or
(ii) an amino acid sequence having at least 75% identity to the amino acid sequence shown in SEQ ID NO:14, wherein one or more of the amino acids corresponding to positions 16 and 17 of SEQ ID NO:14 optionally is not a leucine, and is preferably an alanine or a glycine.

Embodiment 10. The fusion protein of embodiment 8 or 9, wherein the effector domain comprises a BRS from CD3ε, or a functional portion or variant thereof.

Embodiment 11. The fusion protein of any one of embodiments 1-10, wherein the effector domain further comprises an ITAM from CD3ζ, or a functional portion or variant thereof.

Embodiment 12. The fusion protein of embodiment 11, wherein the effector domain further comprises two or three of ITAMs (a), (b), and (c) from CD3ζ, or functional portions or variants thereof.

Embodiment 13. The fusion protein of embodiment 11 or 12, wherein the effector domain comprises an amino acid sequence having at least 75% identity to the amino acid sequence shown in SEQ ID NO:17.

Embodiment 14. The fusion protein of any one of embodiments 1-13, further comprising a costimulatory domain or a functional portion or variant thereof.

Embodiment 15. The fusion protein of embodiment 14, wherein the costimulatory domain or functional portion or variant thereof is from 4-1BB, CD28, OX40, CD27, CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or any combination thereof.

Embodiment 16. The fusion protein of embodiment 15, wherein the costimulatory domain comprises a costimulatory domain, or a functional portion or variant thereof, from 4-1BB.

Embodiment 17. The fusion protein of any one of embodiments 14-16, wherein the intracellular domain comprises a costimulatory domain, an endodomain or effector domain from CD3ζ or a functional portion or variant thereof, and an effector domain from CD3ε or a functional portion or variant thereof, wherein the effector domain from CD3ε or functional portion or variant thereof optionally comprises or consists essentially of a PRS and an ITAM from CD3ε.

Embodiment 18. The fusion protein of embodiment 17, wherein the effector domain from CD3ε or functional portion or variant thereof is disposed between the costimulatory domain and the endodomain or effector domain from CD3ζ or functional portion or variant thereof.

Embodiment 19. The fusion protein of embodiment 17, wherein the endodomain or effector domain from CD3ζ or functional portion or variant thereof is disposed between the costimulatory domain and the effector domain from CD3ε or functional portion or variant thereof.

Embodiment 20. The fusion protein of any one of embodiments 1-19, wherein the transmembrane domain comprises or is a CD28 transmembrane domain, a CD27 transmembrane domain, a CD4 transmembrane domain, a CD8 transmembrane domain, or any combination thereof.

Embodiment 21. The fusion protein of any one of embodiments 1-20, wherein the extracellular domain comprises a CH1, a CH2, a CH3, a CL, a CD8 extracellular domain, a CD28 extracellular domain, a CD4 extracellular domain, an IgG hinge, a glycine-serine linker comprising from about one to about ten repeats of GlyₓSer_{y} (SEQ ID NO:108), a linker having an amino acid sequence as set forth in any one of SEQ ID NOs.:103-107, 109, or 110, or a functional variant or portion thereof, or a combination thereof.

Embodiment 22. The fusion protein of any one of embodiments 1-21, wherein the extracellular domain comprises a linker disposed between the binding domain and the transmembrane domain.

Embodiment 23. The fusion protein of embodiment 22, wherein the linker comprises a hinge region or a portion thereof.

Embodiment 24. The fusion protein of any one of embodiments 1-23, wherein the binding domain comprises or consists of a scFv, a Fab, a scTCR, or a ligand.

Embodiment 25. The fusion protein of embodiment 23, wherein the binding domain comprises a scFv.

Embodiment 26. The fusion protein of any one of embodiments 1-25, wherein the binding domain is chimeric, human, or humanized.

Embodiment 27. The fusion protein of any one of embodiments 1-26, wherein the binding domain specifically binds to an antigen that is expressed by or is associated with a cancer.

Embodiment 28. The fusion protein of embodiment 27, wherein the cancer comprises a solid tumor.

Embodiment 29. The fusion protein of any one of embodiments 1-28, wherein the antigen is selected from a ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, ERBB2, BRAFV600E, MAGE-A3, MAGE-A4, SSX-2, PRAME, HA-1, CD79a, CD79b, SLAMF7, or CEA antigen.

Embodiment 30. The fusion protein of embodiment 29, wherein the antigen is a ROR1 antigen.

Embodiment 31. The fusion protein of any one of embodiments 1-30, further comprising a protein tag.

Embodiment 32. The fusion protein of any one of embodiments 1-31, wherein the fusion protein, when expressed by a host cell, optionally an immune system cell, provides for or promotes:
(i) improved cell signaling, and/or activity in response to antigen relative to a host cell expressing a reference fusion protein, wherein improved cell signaling optionally comprises increased and/or sustained cytokine production and/or release, and/or phosphorylation of one or more protein associated with an immune cell response to antigen-binding, or the like, such as LAT, PLC-γ1, SLP-76, or any combination thereof;
(ii) improved cell activity in response to antigen relative to a host cell expressing a reference fusion protein, wherein improved cell signaling optionally comprises increased mobilization of intracellular calcium, killing activity, proliferation, earlier activation in response to antigen, or any combination thereof,
(iii) improved cell signaling and/or activity, relative to a host cell expressing a reference fusion protein, upon binding to a target antigen that is expressed at a low level or an intermediate level on a target cell surface;
(iv) reducing or suppressing growth, area, volume, and/or spread of a tumor that expresses an antigen that is recognized and/or specifically bound by the fusion protein, of killing tumor cells, and/or of increasing survival of the subject to a greater degree and/or for a longer period of time as compared to a reference subject administered a host cell expressing a reference fusion protein;
(iv) more efficient phosphorylation of LAT, SLP-76, and/or PLC-γ1 as compared to a reference fusion protein expressed by a host cell;
(v) improved sensitivity to antigen as compared to a host cell expressing a reference fusion protein, but does not produce more, or substantially more, of a pro-inflammatory cytokine as compared to the host cell expressing the reference fusion protein; or
(vi) any combination of (i)-(v).

Embodiment 33. The fusion protein of embodiment 32, wherein the intracellular component comprises a costimulatory domain, or a functional portion or variant thereof, from 4-1BB.

Embodiment 34. The fusion protein of embodiment 32 or 33, wherein the reference protein comprises an intracellular domain comprising a 4-1BB costimulatory domain and/or a CD28 costimulatory domain.

Embodiment 35. The fusion protein of any one of embodiments 32-34, wherein the fusion protein and the reference protein each comprise an endodomain or effector domain from CD3ζ or a functional portion or variant thereof.

Embodiment 36. The fusion protein of any one of embodiments 32-35, wherein the host cell expressing the fusion protein and the host cell expressing the reference fusion protein are each a T cell, optionally a CD8+ T cell.

Embodiment 37. An isolated polynucleotide, encoding the fusion protein of any one of embodiments 1-36.

Embodiment 38. The isolated polynucleotide of embodiment 37, further comprising a polynucleotide encoding a transduction marker.

Embodiment 39. The isolated polynucleotide of embodiment 38, wherein the encoded transduction marker comprises EGFRt, CD19t, CD34t, or NGFRt.

Embodiment 40. The isolated polynucleotide of any one of embodiments 38 or 39, further comprising a polynucleotide encoding a self-cleaving polypeptide.

Embodiment 41. The isolated polynucleotide of embodiment 40, wherein the fusion protein-encoding polynucleotide is separated from the transduction marker-encoding polynucleotide by the polynucleotide encoding a self-cleaving polypeptide.

Embodiment 42. The isolated polynucleotide of embodiment 40 or 41, wherein the encoded self-cleaving polypeptide comprises a P2A, an F2A, a T2A, an E2A, or a variant thereof.

Embodiment 43. The isolated polynucleotide of any one of embodiments 37-42, wherein the polynucleotide is codon optimized for expression in a host cell.

Embodiment 44. The isolated polynucleotide of any one of embodiments 37-43, wherein the polynucleotide comprises a polynucleotide having at least 70% identity to the nucleotide sequence set forth in any one of SEQ ID NOs.:34-46.

Embodiment 45. An expression vector, comprising the isolated polynucleotide of any one of embodiments 37-44 operably linked to an expression control sequence.

Embodiment 46. The expression vector of embodiment 45, wherein the vector is capable of delivering the polynucleotide to a host cell.

Embodiment 47. The expression vector of embodiment 46, wherein the host cell is a hematopoietic progenitor cell or a human immune system cell.

Embodiment 48. The expression vector of embodiment 47, wherein the human immune system cell is a CD4+ T cell, a CD8+ T cell, a CD4− CD8− double negative T cell, a γδ T cell, a natural killer cell, a natural killer T cell, a dendritic cell, or any combination thereof.

Embodiment 49. The expression vector of embodiment 47 or 48, wherein the T cell is a naïve T cell, a central memory T cell, a stem cell memory T cell, an effector memory T cell, or any combination thereof.

Embodiment 50. The expression vector of any one of embodiments 45-49, wherein the vector is a viral vector.

Embodiment 51. The expression vector of embodiment 50, wherein the viral vector is a lentiviral vector or a γ-retroviral vector.

Embodiment 52. A host cell, comprising the polynucleotide of any one of embodiments 37-44.

Embodiment 53. A host cell, expressing at its cell surface the fusion protein of any one of embodiments 1-36.

Embodiment 54. The host cell of embodiment 53, further expressing a transduction marker at its cell surface.

Embodiment 55. The host cell of any one of embodiments 52-54, wherein the host cell is a hematopoietic progenitor cell or a human immune system cell.

Embodiment 56. The host cell of embodiment 55, wherein the human immune system cell is a CD4+ T cell, a CD8+ T cell, a CD4− CD8− double negative T cell, a γδ T cell, a natural killer cell, a natural killer T cell, a dendritic cell, or any combination thereof.

Embodiment 57. The host cell of embodiment 55 or 56, wherein the host cell comprises a T cell.

Embodiment 58. The host cell of embodiment 56 or 57, wherein the T cell is a naïve T cell, a central memory T cell, a stem cell memory T cell, an effector memory T cell, or any combination thereof.

Embodiment 59. The host cell of any one of embodiments 52-58, comprising a chromosomal gene knockout or a mutation of a PD-1 gene; a LAG3 gene; a TIM3 gene; a CTLA4 gene; an HLA component gene; a TCR component gene, or any combination thereof.

Embodiment 60. A composition, comprising a fusion protein of any one of embodiments 1-36 and a pharmaceutically acceptable carrier, excipient, or diluent.

Embodiment 61. A composition, comprising a host cell of any one of embodiments 52-59, and a pharmaceutically acceptable carrier, excipient, or diluent.

Embodiment 62. A unit dose, comprising an effective amount of the host cell of any one of embodiments 52-59, or of the host cell composition of embodiment 61.

Embodiment 63. The unit dose of embodiment 62, comprising (i) a composition comprising at least about 30% CD4+ T host cells, combined with (ii) a composition comprising at least about 30% CD8+ T host cells, in about a 1:1 ratio.

Embodiment 64. A method of treating a disease or condition in a subject, the method comprising administering to the subject an effective amount of the host cell of any one of embodiments 52-59, the composition of embodiment 60 or 61, or the unit dose of embodiment 62 or 63, wherein the disease or condition is characterized by the presence of the antigen.

Embodiment 65. A method of eliciting an immune response against an antigen that is specifically bound by the fusion protein of any one of embodiments 1-36, the method comprising administering to a subject comprising or expressing the antigen an effective amount of the host cell of any one of embodiments 52-59, the composition of embodiment 60 or 61, or the unit dose of embodiment 62 or 63.

Embodiment 66. The method of embodiment 64, wherein the disease or condition comprises a hyperproliferative disease or a proliferative disease.

Embodiment 67. The method of embodiment 64 or 66, wherein the disease or condition is a cancer.

Embodiment 68. The method of embodiment 67, wherein the cancer comprises a carcinoma, a sarcoma, a glioma, a lymphoma, a leukemia, a myeloma, or any combination thereof.

Embodiment 69. The method of embodiment 67 or 68, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

Embodiment 70. The method of any one of embodiments 67-69, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

Embodiment 71. The method of any one of embodiments 67-70, wherein the cancer comprises a solid tumor.

Embodiment 72. The method of embodiment 71, wherein the solid tumor is a sarcoma or a carcinoma.

Embodiment 73. The method of embodiment 72, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

Embodiment 74. The method of embodiment 72 or 73, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

Embodiment 75. The method of embodiment 72 or 73, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Embodiment 76. The method of any one of embodiments 64-75, wherein the host cell is an allogeneic cell, a syngeneic cell, or an autologous cell.

Embodiment 77. The method of any one of embodiments 64-76, wherein the method comprises administering a plurality of unit doses to the subject.

Embodiment 78. The method of embodiment 77, wherein the plurality of unit doses are administered at intervals between administrations of about two, three, four, five, six, seven, eight, or more weeks.

Embodiment 79. The method according to any one of embodiments 64-78, wherein the unit dose comprises about 10⁵ cells/m² to about 10¹¹ cells/m².

Embodiment 80. The method of any one of embodiments 57-79, wherein the subject is receiving, has received, or will receive one or more of:
(i) chemotherapy;
(ii) radiation therapy;
(iii) an inhibitor of an immune suppression component
(iv) an agonist of a stimulatory immune checkpoint agent;
(v) RNAi;
(vi) a cytokine;
(vii) a surgery;
(viii) a monoclonal antibody and/or an antibody-drug conjugate; or
(ix) any combination of (i)-(viii), in any order.

Embodiment 81. Use of the polynucleotide of any one of embodiments 37-44, or the expression vector of any one of embodiments 45-51, the host cell of any one of embodiments 52-59, or the composition of any one of embodiments 60 or 61, in the manufacture of a medicament for the treatment of a disease; e.g., a disease characterized by or associated with expression of an antigen that is specifically bound by the fusion protein of any one of embodiments 1-36.

Embodiment 82. Use of the polynucleotide of any one of embodiments 37-44, or the expression vector of any one of embodiments 45-51, the host cell of any one of embodiments 52-59, or the composition of any one of embodiments 60 or 61, in the treatment of a disease; e.g., a disease characterized by or associated with expression of an antigen that is specifically bound by the fusion protein of any one of embodiments 1-36.

Embodiment 83. The use according to embodiment 81 or 82, wherein the disease is a cancer.

Embodiment 84. A fusion protein, comprising:
(a) an extracellular component comprising a binding domain that specifically binds to an antigen;
(b) a transmembrane domain; and
(c) an intracellular component comprising an effector domain or a functional portion thereof, wherein the effector domain or functional portion or variant thereof comprises:
(i) an Intracellular Tyrosine-based Activation Motif (ITAM) from CD3ε, or a functional portion or variant thereof;
(ii) a Proline Rich Sequence (PRS) from CD3ε, or a functional portion or variant thereof; or
(iii) both of (i) and (ii),
wherein
(1) the extracellular domain does not comprise a TCR ectodomain; and/or
(2) the fusion protein does not associate with or form a TCR complex when expressed by a T cell.

Embodiment 85. A fusion protein, comprising:
(a) an extracellular component comprising a binding domain that specifically binds to an antigen;
(b) a transmembrane domain; and
(c) an intracellular component comprising an effector domain or a functional portion thereof, wherein the effector domain or functional portion thereof comprises:
(i) an Intracellular Tyrosine-based Activation Motif (ITAM) from CD3ε, or a functional portion or variant thereof;
(ii) a Proline Rich Sequence (PRS) from CD3ε, or a functional portion or variant thereof; or
(iii) both of (i) and (ii).

Embodiment 86. The fusion protein of embodiment 84 or 85, wherein the intracellular component comprises an ITAM from CD3ε and a PRS from CD3ε, and further comprises an endodomain or effector domain from CD3ζ or functional portion or variant thereof.

Embodiment 87. The fusion protein of embodiment 86, wherein the ITAM from CD3ε comprises or consists of the amino acid sequence set forth in SEQ ID NO:6.

Embodiment 88. The fusion protein of embodiment 86 or 87, wherein the PRS from CD3ε comprises or consists of the amino acid sequence set forth in SEQ ID NO:7 or 8.

Embodiment 89. The fusion protein of any one of embodiments 85-88, wherein the intracellular component further comprises a costimulatory domain, or a functional portion or variant thereof, from 4-1BB.

Embodiment 90. The fusion protein of embodiment 89, wherein the intracellular component comprises or consists of any amino acid sequence having at least 75%, 80%, 85%, 90%, 91%, 92%, 93%, 94%, 95%, 96%, 97%, 98%, or 99%, to the amino acid sequence set forth in any one of SEQ ID NOs:6-8, 24, 25, or 27-32.

Embodiment 91. An isolated polynucleotide encoding the fusion protein of any one of embodiments 85-90.

Embodiment 92. A host cell, comprising the polynucleotide of embodiment 91, wherein the host cell is optionally an immune system cell, further optionally a T cell.

### EXAMPLES

### Example 1

### Analysis and Comparison of TCR- and CAR-Induced Phosphoprotein Signaling

CD19-specific CAR T cells are a FDA-approved therapy for individuals with relapsed or refractory B cell malignancies. Generally, greater than 70% of acute lymphocytic leukemia patients who are treated with CAR T cells achieve a complete response (Majzner & Mackall, Cancer Discov. 8:1219-1226 (2018)). However, a significant proportion relapse with leukemia blasts expressing little to no detectable CD19 (see, e.g., Turtle et al., Sci. Transl. Med. 8:355ra116 (2016); Park et al., N. Engl. J. Med. 378:449-459 (2018); Maude et al., N. Engl. J. Med. 378:439-448 (2018); Fry et al., Nature Medicine 24:20-28 (2018)). A clinical trial of BCMA-specific CAR T cells in relapsed or refractory multiple myeloma also demonstrates that outgrowth of BCMA-negative myeloma cells is a potential cause of relapse (Brudno et al., J. Clin. Oncol. 36.22: 2267-80 (2018)). In some cases, solid epithelial tumors do not homogenously or highly express lineage defining markers like CD19 or BCMA, but can share lineage defining markers with normal tissues. Addressing antigen downregulation and loss of tumor-specific antigens is an important objective to for obtaining complete and durable responses to CAR T cell therapies.

TCRs can detect peptide antigens presented by MHC molecules with high sensitivity. It has been reported that as few as one to ten agonist pMHC complexes in a sea of thousands of self-pMHC can trigger T cell activation through the TCR (e.g., Sykulev et al., Immunity 4:565-571 (1996); Huang et al., Immunity 39:846-857 (2013)). In contrast, it has been reported that CARs require at least 200 molecules of antigen on the target cell to initiate CAR T cell cytotoxicity and thousands of molecules to elicit full effector function (e.g., Watanabe et al., J. Immunol. 194:911-920 (2015); Walker et al., Mol. Ther. 25:2189-2201 (2017); Friedman et al., Hum. Gene Ther. 29:585-601 (2018)). Whether these requirements for increased antigen density apply to all CARs or can be improved upon is an open question.

In general, other functional differences between naturally occurring TCRs and CARs include higher binding affinity of CARs for antigen (see, e.g., Hogquist & Jameson, Nat. Immunol. 15:815-823 (2014); Chmielewski et al., J. Immunol. 173:7647-7653 (2004); and Hudecek et al., Clin. Cancer Res. 19:3153-3164 (2013)), which may have implications for receptor off-rate and repeated triggering. Additionally, TCRs are thought to form complexes with multiple CD3 proteins following binding to antigen:MHC and associate with various downstream signaling proteins, while CARs generally contain only a CD3ζ signaling endodomain in a single molecule comprising a binding domain, a transmembrane domain, and a costimulatory domain from CD28, 4-1BB, or ICOS. In the case of CARs, variation in the stoichiometric ratios of signaling molecules or the absence of certain signaling components at the immunological synapse may alter antigen sensitivity and signal intensity. Indeed, initial images of CAR immunological synapses were distinct from those formed by TCRs (see James & Vale, Nature 487:64-69 (2012)).

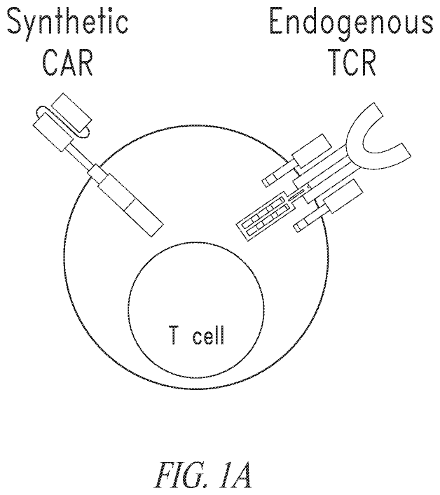

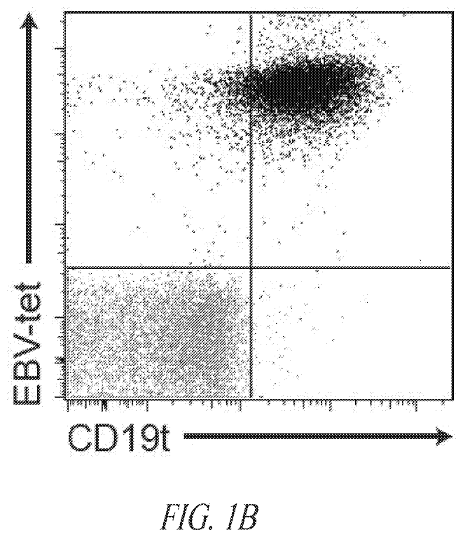

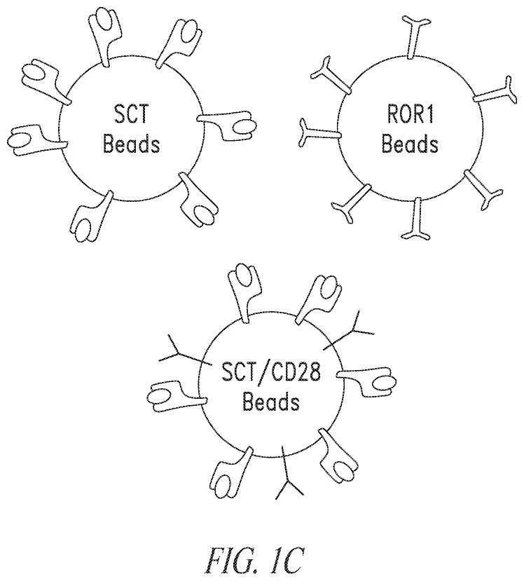

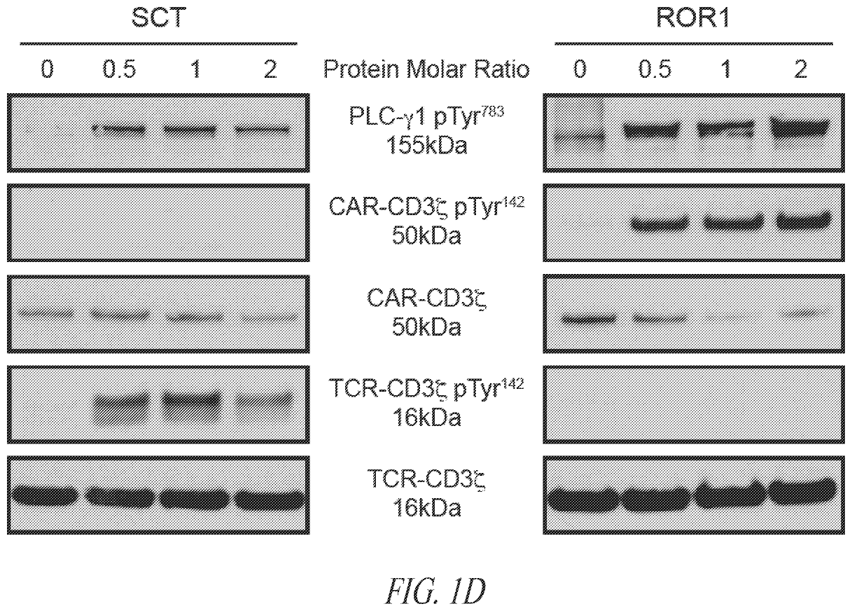

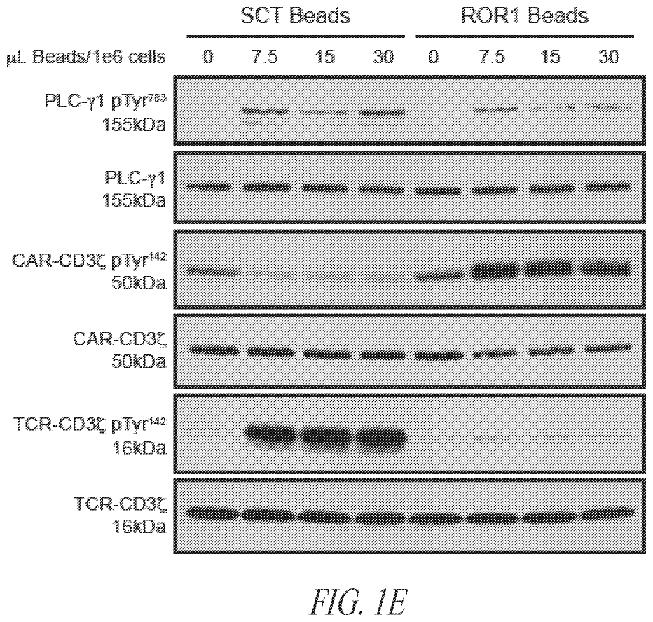

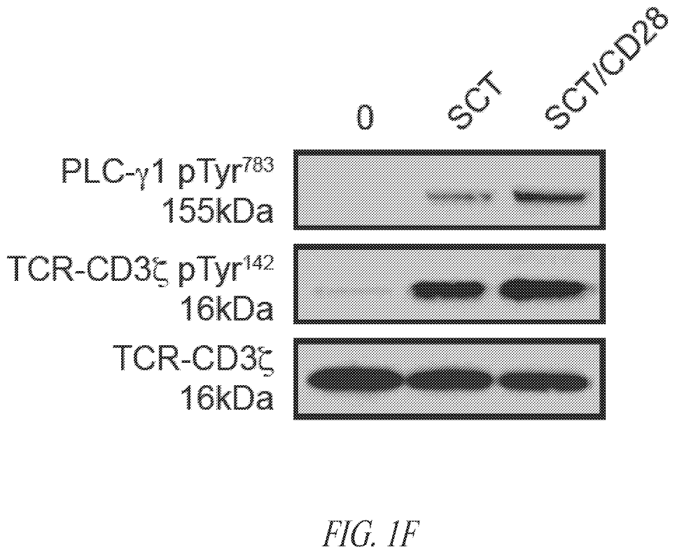

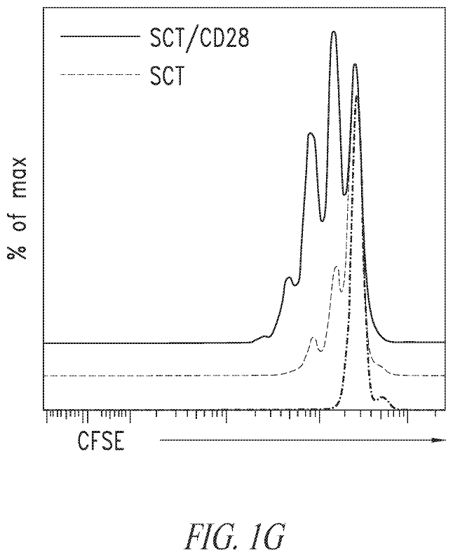

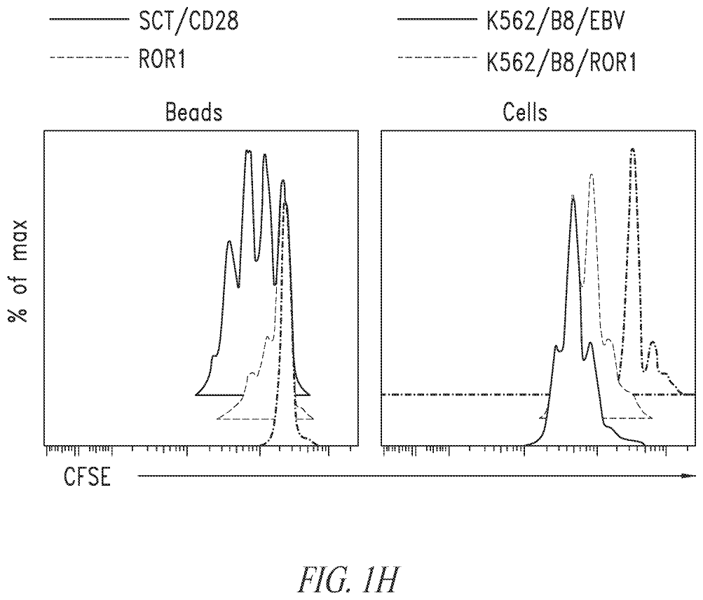

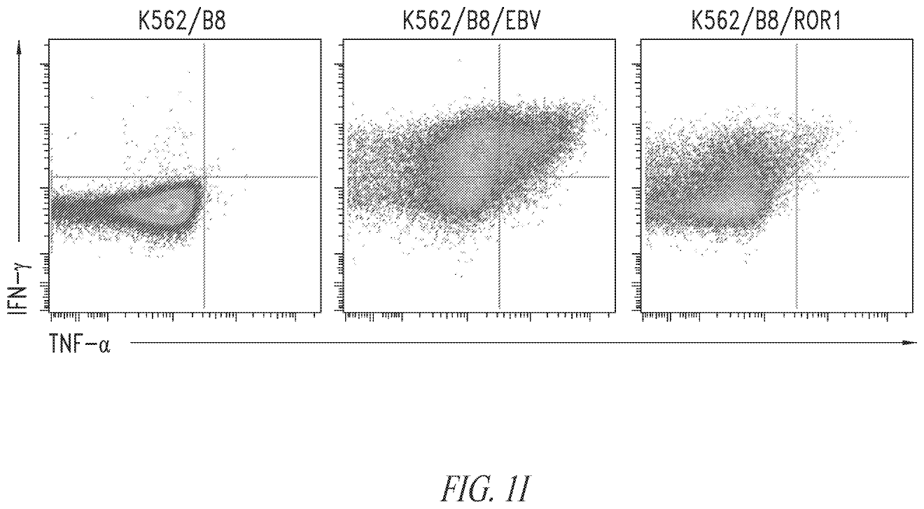

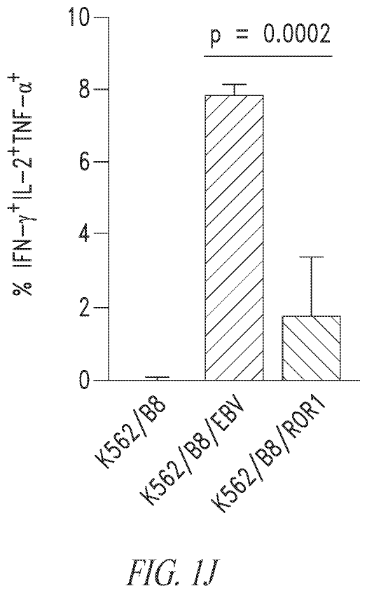

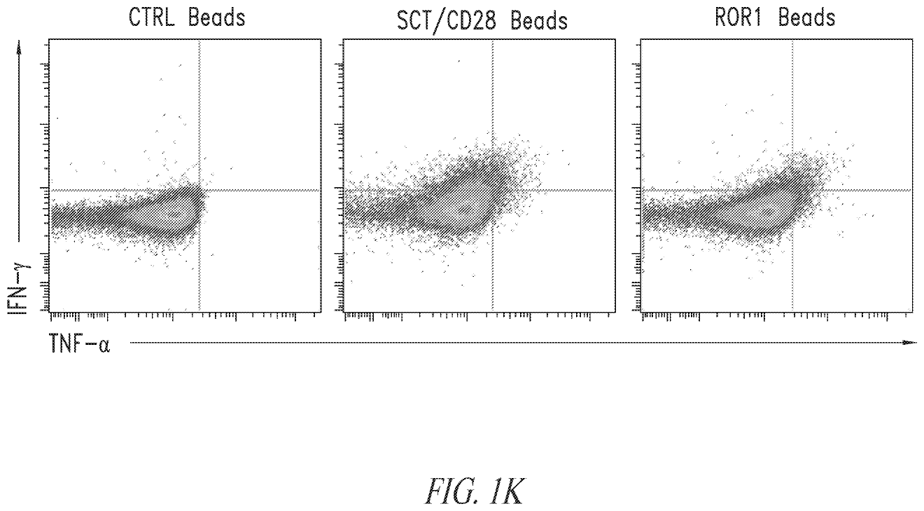

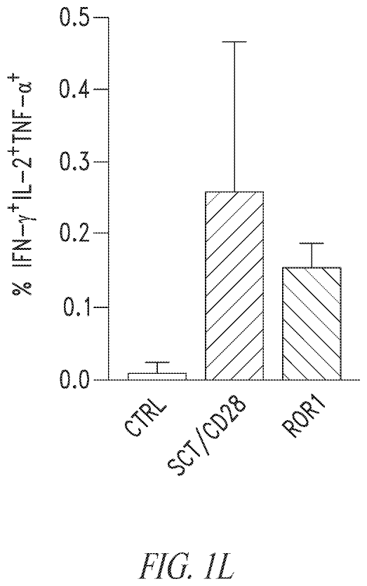

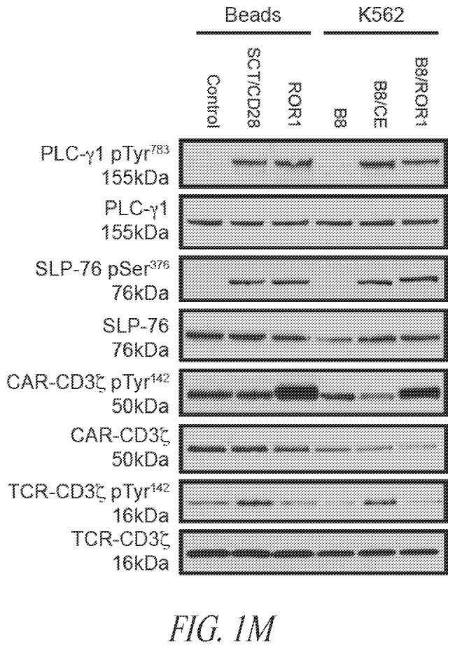

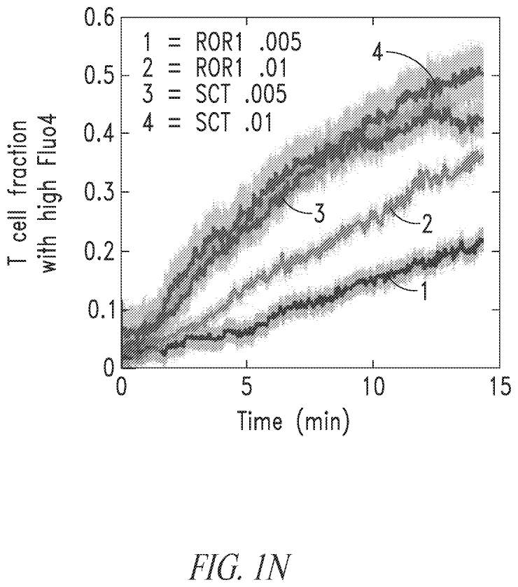

To comprehensively compare TCR and CAR ligation-induced phosphoprotein signaling events, a mass spectrometry (MS) platform was developed to analyze TCR or CAR signaling in a single population of primary T cells possessing a mono-specific TCR or CAR. Bi-specific T cells were engineered that endogenously express an EBV-specific TCR and were transduced to express a ROR1-specific CAR (FIG. 1A). Bi-specific T cells offer the advantages of: 1) comparing TCR and CAR function in one cell population with a single culture history and phenotype, and 2) utilizing the endogenous TCR, which negates potential effects of reduced TCR expression or TCRα and TCRβ chain mispairing after transduction of an engineered TCR. Briefly, CD8⁺CD45RO⁺ memory T cells from HLA-B8⁺ EBV seropositive donors were stimulated with autologous irradiated and EBV peptide-pulsed peripheral blood mononuclear cells. Dividing cells were then transduced with a lentiviral vector encoding a ROR1-specific CD28/CD3ζ CAR. Bi-specific T cells that bound to an HLA-B8/EBV-RAK tetramer and expressed the CAR transduction marker, tCD19, were purified by fluorescence-activated cell sorting (FACS) and expanded (FIG. 1B).

Stimulating EBV/ROR1 bi-specific T cells with tumor cells expressing TCR or CAR antigens was an attractive strategy to initiate phosphoprotein signaling, but antigen-presenting cells contaminated the cellular lysate and were not amenable for MS analysis (data not shown). Therefore, microbeads coated with recombinant single chain timers (SCT) of EBV RAKFKQLL peptide, HLA-B8, and 32 microglobulin, or recombinant ROR1 ectodomain were formulated and used to initiate cell signaling (FIG. 1C) (see Yu et al., J. Immunol. 168:3145-3149 (2002)). SCT or ROR1 beads activated T cell signaling and the amount of protein coated on the beads was titrated down (FIG. 1D), as was as the bead to cell ratio (FIG. 1E) to levels that reduced reagent costs while still saturating PO₄ of the CAR, TCR, and PLC-γ1. A CD28 mAb was also added to SCT beads so that TCR bead-based stimulation delivered ‘signal 1’ and ‘signal 2’ and would be functionally equivalent to CD28/CD3ζ CAR stimulation. Consistent with the known additive role of CD28 costimulation, SCT/CD28 beads increased PLC-γ1 Tyr⁷⁸³ PO₄ and T cell proliferation after stimulation compared to SCT beads (FIGS. 1F, 1G).

To determine whether microbead-based TCR or CAR stimulation could effectively substitute for cell-based stimulation, T cell effector functions and signaling profiles initiated by beads or cells were compared. Expanded bi-specific T cells were incubated with either SCT/CD28 or ROR1 microbeads, or K562 cells expressing HLA-B8 alongside endogenously processed EBV peptides or full-length human ROR1. TCR stimulation using either SCT/CD28 beads or K562/B8/EBV cells elicited greater cell division than CAR stimulation using either ROR1 beads or K562/B8/ROR1 cells (FIG. 1H). TCR stimulation with either beads or cells also led to greater frequencies of cells producing IFN-γ, IL-2, and TNF-α than CAR stimulation (FIGS. 11-1L). To further validate a bead-based approach, PO₄ of key signaling intermediates was measured in in bi-specific T cells after 45 minutes of stimulation with K562 APC or ligand-coated beads. PO₄ of native CD3ζ or exogenous CAR-CD3 (chains at Tyr¹⁴² appeared nearly identical after antigen presentation by APC or beads, and PO₄ of SLP-76 Ser³⁷⁶ and PLC-γ1 Tyr⁷⁸³ were highly similar (FIG. 1M). Taken together, these results confirmed the ability of microbeads to substitute for K562 antigen-presenting cells, although the overall magnitude of the effector responses after bead stimulation were more modest, possibly due to the absence of adhesion and costimulatory molecules on the magnetic beads that were present on K562 cells.

Fluorescence microscopy using the Ca²⁺-sensitive dye Fluo-4 AM was also used to measure Ca²⁺ flux after antigen engagement and provide an index of the relative antigen sensitivity of the TCR and CAR. Recombinant SCT and ROR1 ectodomain were biotinylated and coated onto a supported lipid bilayer via biotin/streptavidin linkage. The responses of hundreds of individual T cells to TCR or CAR stimulation were compared using the fraction of cells exhibiting high intracellular calcium as a metric of the response amplitude. In these experiments, antigen density was modulated by incorporating a small mole fraction (<1%) of biotinylated PE into the supported bilayer, followed by sequential labeling with excess concentrations of streptavidin and biotinylated SCT or ROR1. At two unique antigen densities, a greater fraction of bi-specific T cells was triggered by TCR stimulation than by CAR stimulation (FIG. 1N). At both antigen densities, the TCR response appeared nearly saturated because similar frequencies of T cells responded over time. In contrast, the fraction of triggered T cells diminished as ROR1 density was reduced. Because TCR and CAR triggering were quantified at identical SCT and ROR1 densities, these results demonstrated that TCRs possessed increased antigen sensitivity as compared to a CD28/CD3ζ CAR.

MS Identifies Common Protein Phosphorylation Events after TCR or CAR Stimulation

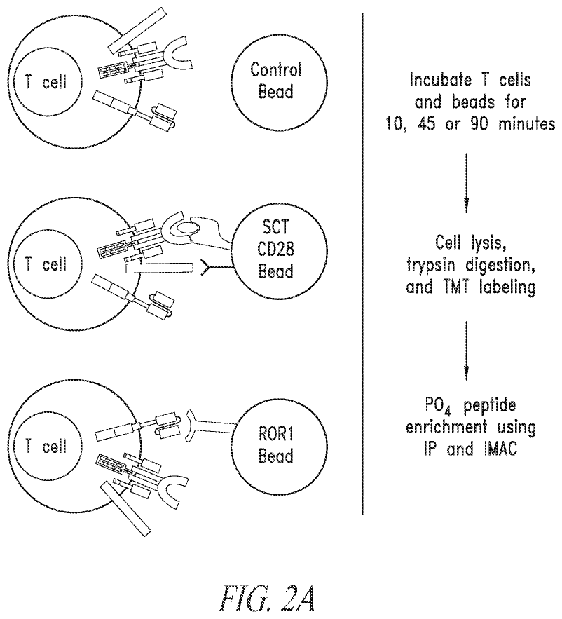

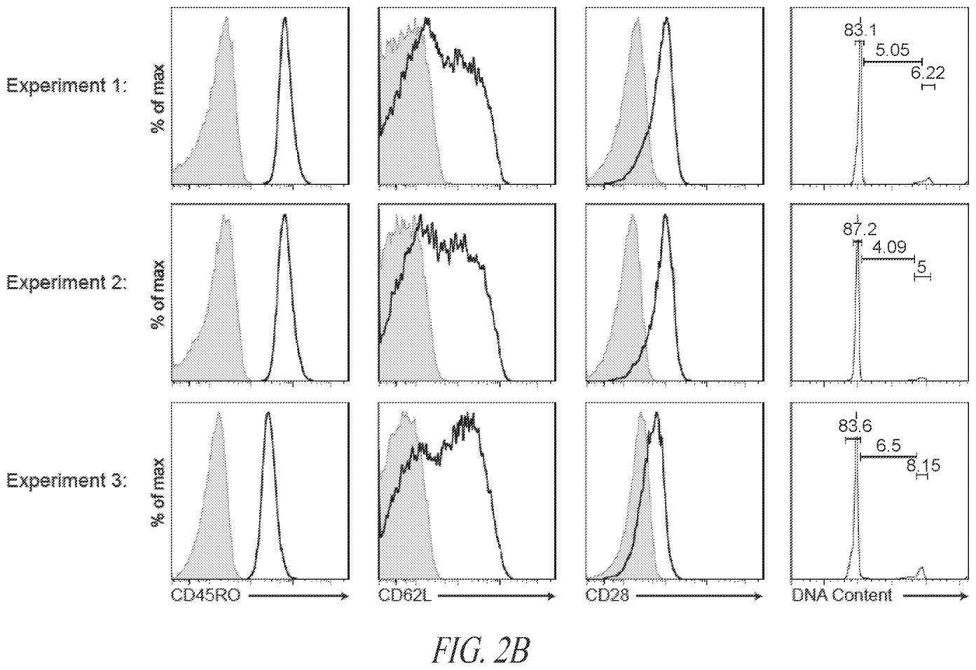

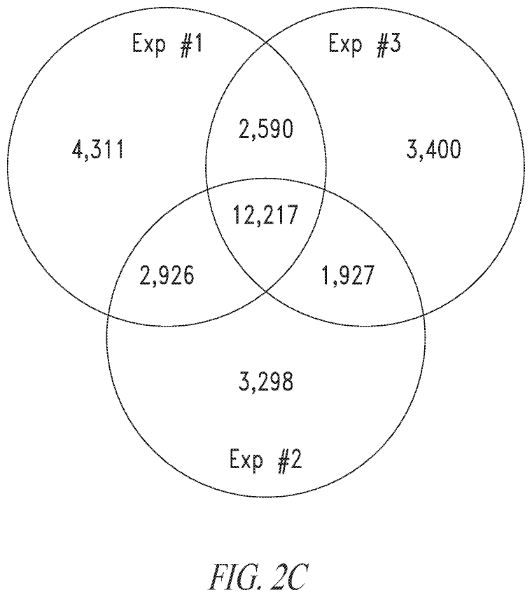

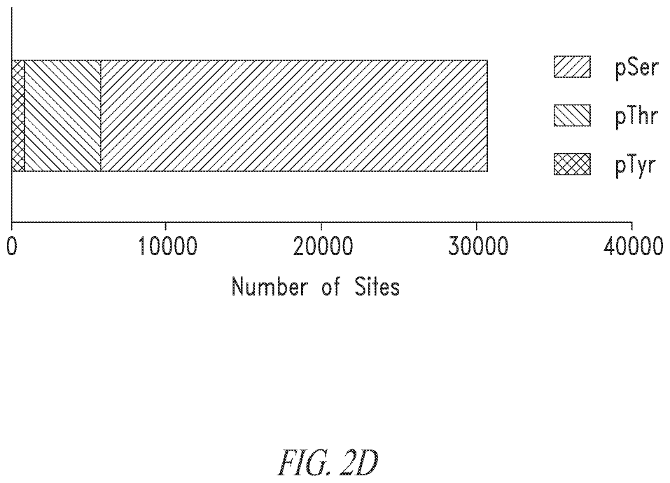

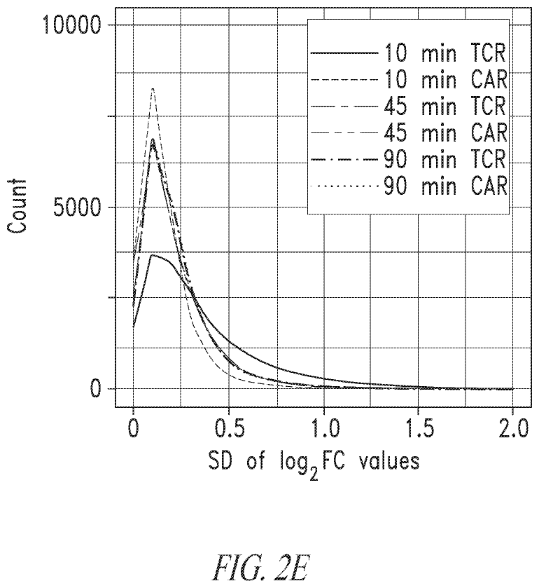

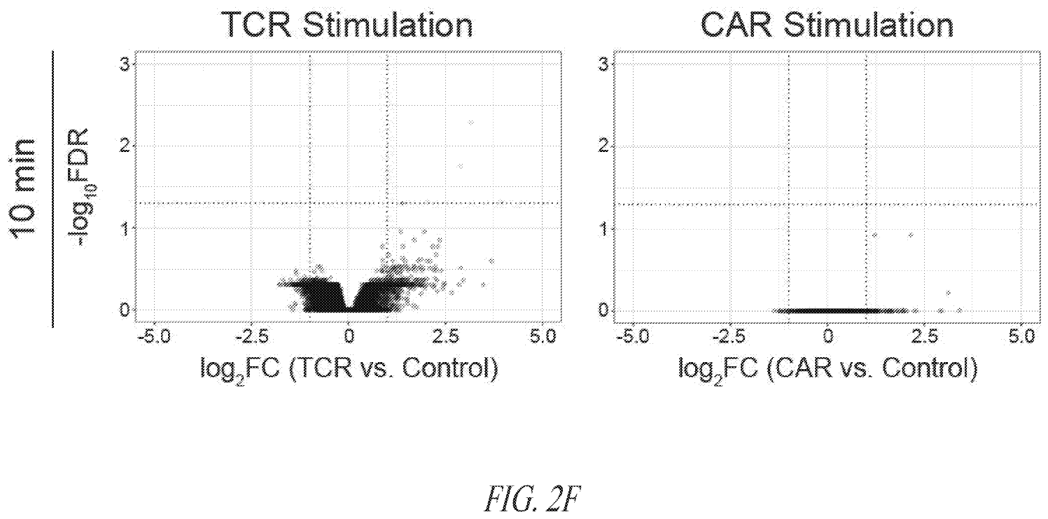

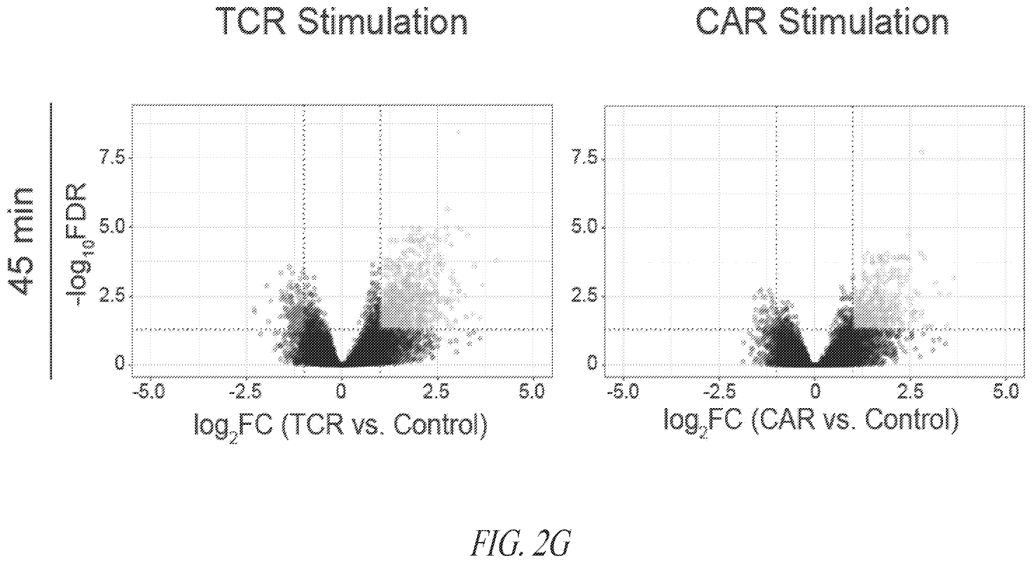

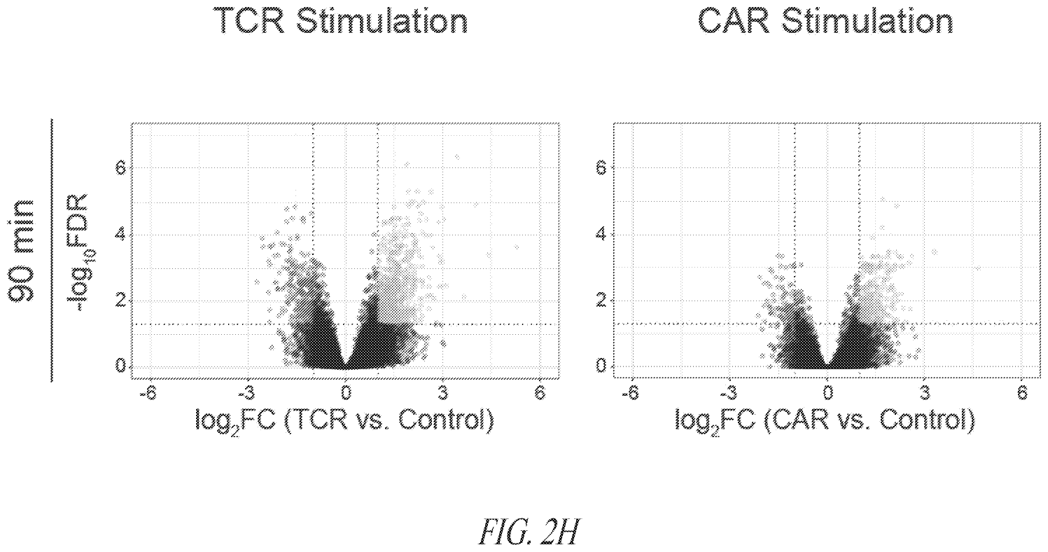

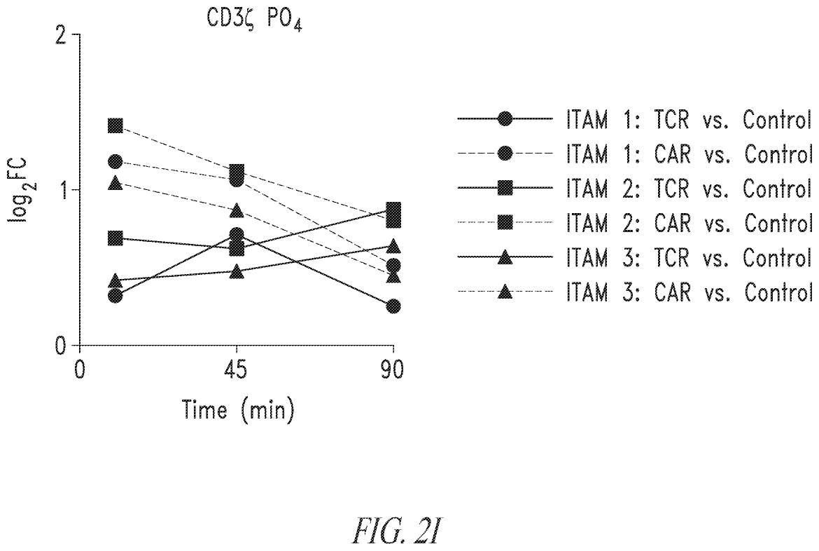

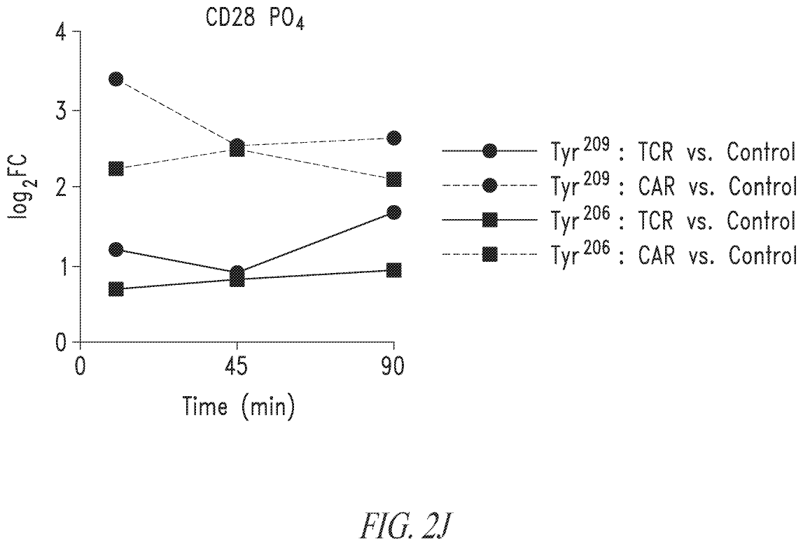

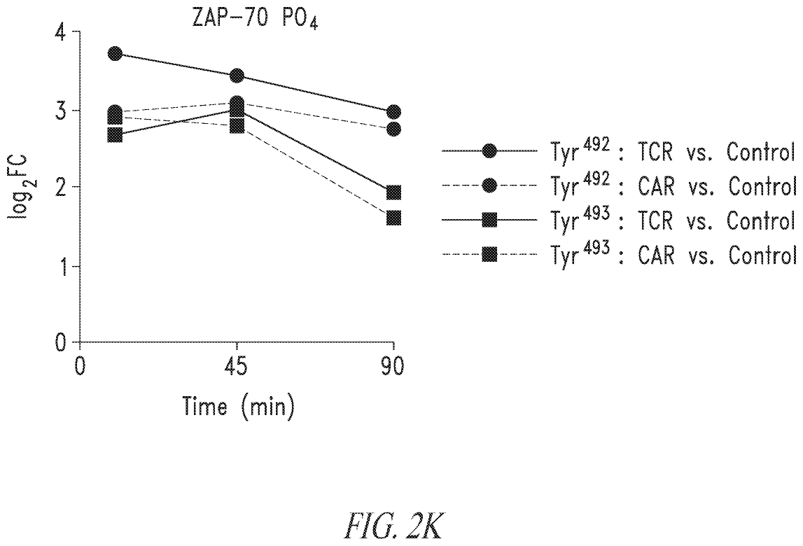

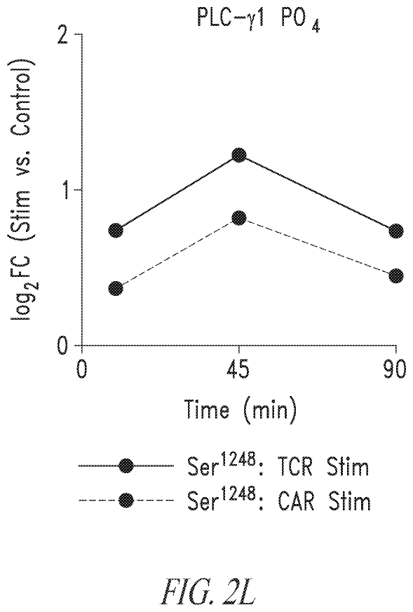

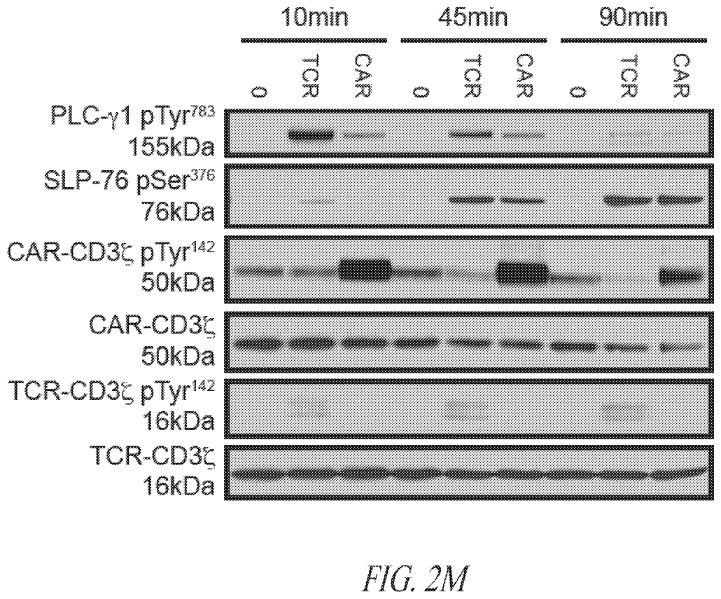

To measure protein PO₄ signaling events initiated by TCR or CAR stimulation by MS, three independent experiments were performed in which EBV/ROR1 bi-specific T cells were incubated with SCT/CD28, ROR1, or uncoated (control) magnetic beads for 10, 45, or 90 minutes prior to cell lysis (FIG. 2A). Bi-specific T cells generated from two different donors were used in two experiments; a third experiment utilized bi-specific T cells independently derived from one of the two donors. Cell phenotyping prior to TCR (SCT/CD28) or CAR (ROR1) stimulation showed that bi-specific T cells expressed CD45RO, CD62L, and CD28, and that >83% of cells were in the G0/G1 cell cycle phase, consistent with a resting memory phenotype (FIG. 2B). Using identical phosphopeptide labeling and enrichment techniques, a total of 30,669 PO₄ sites corresponding to 4,997 gene products were detected from these three experiments (FIG. 2C). Among PO₄ sites, 715 (2.3%) were phosphotyrosines, 5,056 (16.5%) were phosphothreonines, and 24,898 (81.2%) were phosphoserines (FIG. 2D). For each PO₄ site, log 2 fold-change (log 2FC) values comparing TCR stimulation to control treatment or CAR stimulation to control treatment were consistent across experiments (FIG. 2E).

### Phosphorylation Events Mediated by TCRs and CARs Differ in Kinetics and Magnitude

Statistical analysis (limma in the R statistical computing framework) was performed to identify PO₄ sites that were up- or down-modulated by TCR or CAR stimulation. A PO₄ site was determined to be CAR stimulation-responsive if it was detected in at least two of the three experiments, displayed an average |log 2FC|≥1 between stimulated and unstimulated conditions at 10 or 45 minutes, and met a 5% FDR cutoff. Few PO₄ sites met log 2FC and false discovery rate (FDR) cutoffs after 10 minutes of TCR or CAR stimulation, but hundreds of PO₄ sites were up- or down-modulated after 45 or 90 minutes of TCR and CAR stimulation (FIG. 2F; additional data not shown). On a global level, TCR stimulation induced greater fold changes in protein PO₄ at the 10 minute time point compared to CAR stimulation. These differences became less apparent after 45 and 90 minutes of stimulation (FIGS. 2G and 2H, respectively). Network analysis of stimulation-responsive PO₄ sites revealed that both TCR and CAR stimulation altered the PO₄ of proteins in the KEGG T cell receptor signaling, RNA transport, and actin cytoskeleton regulatory pathways (data not shown).

To confirm that bead stimulation activated TCR and CAR signaling intermediates, the dataset was interrogated for PO₄ sites on CD3ζ, CD28, ZAP-70, and PLC-γ1. PO₄ of all three CD3ζ ITAMs, CD28 tyrosine residues, ZAP-70 regulatory sites, and PLC-γ1 Ser¹²⁴⁸ increased after TCR or CAR stimulation (FIGS. 2I-2L, respectively). CD3ζ and CD28 were more intensely phosphorylated by CAR stimulation than TCR stimulation at the 10 and 45 minute time points. However, the more robust PO₄ of CD3ζ and CD28 after CAR stimulation did not translate into substantially greater ZAP-70 PO₄ as ZAP-70 PO₄ sites displayed similar increases in PO₄ after TCR and CAR stimulation (FIG. 2K). PLC-γ1 Ser¹²⁴⁸ was also more weakly phosphorylated by CAR than TCR stimulation (FIG. 2L). Thus, it appeared that CAR stimulation led to more intense CAR PO₄, but that this strong signal intensity was not as efficiently converted into ZAP-70 PO₄ as after TCR stimulation. These findings were surprising, since the more intense CD3ζ and CD28 PO₄ after CAR stimulation might be expected to result in increased PLC-γ1 PO₄.

CD3ζ and CD28 proteins in bi-specific T cells are encoded by both the endogenous genes and the CAR transgene, and LC-MS/MS cannot differentiate identical peptides in trypsin digested lysates that are derived from distinct sources. Because the CAR protein is larger than endogenous CD3ζ, Western blot analysis was performed on analyzed undigested whole cell lysates. These experiments confirmed selective TCR and CAR activation as well as more intense PO₄ of the CAR CD3ζ domain after CAR ligation than the TCR CD3ζ domain after TCR ligation (FIG. 2M). In contrast, SLP-76 Ser³⁷⁶ and PLC-γ1 Tyr⁷⁸³ were more intensely phosphorylated after TCR stimulation than after CAR stimulation. These data demonstrate that TCR stimulation more efficiently converts small amounts of CD3ζ PO₄ into intense ZAP-70, SLP-76, and PLC-(1 PO₄ and suggest that CAR stimulation requires greater receptor PO₄ to initiate intracellular signaling than TCR stimulation.

### CAR Stimulation Promotes Less Intense PO₄ of CD3 Chains and Signaling Adaptors

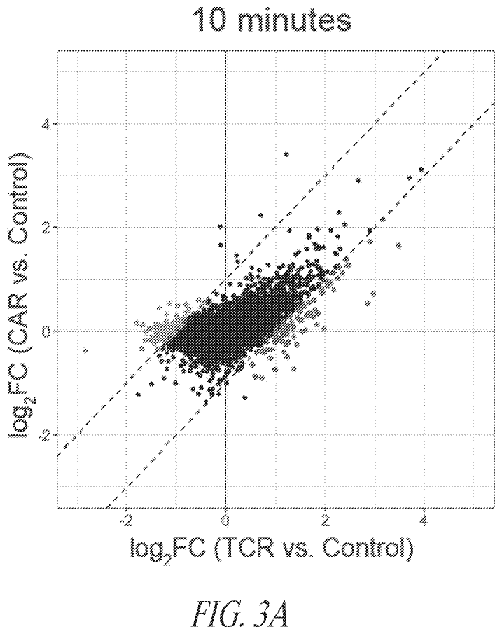

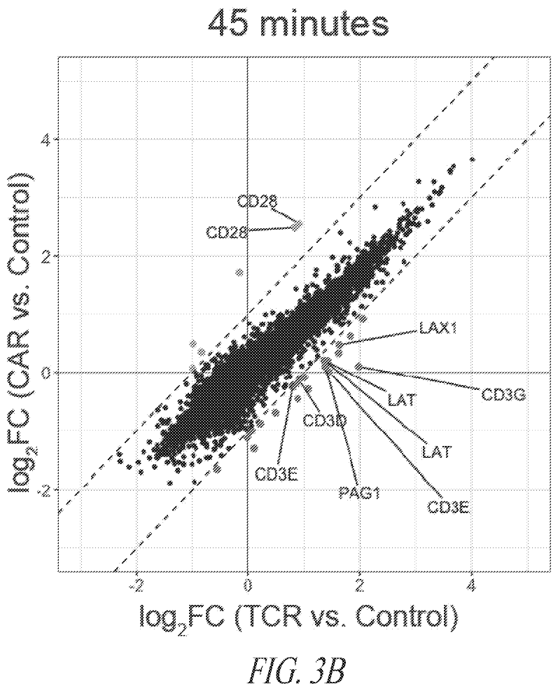

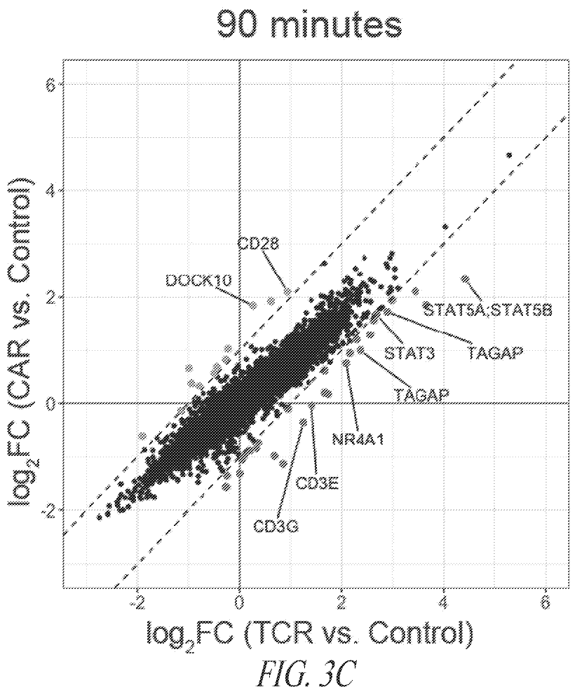

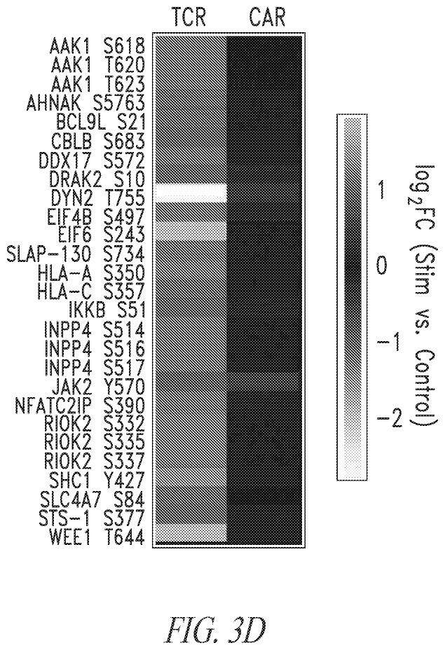

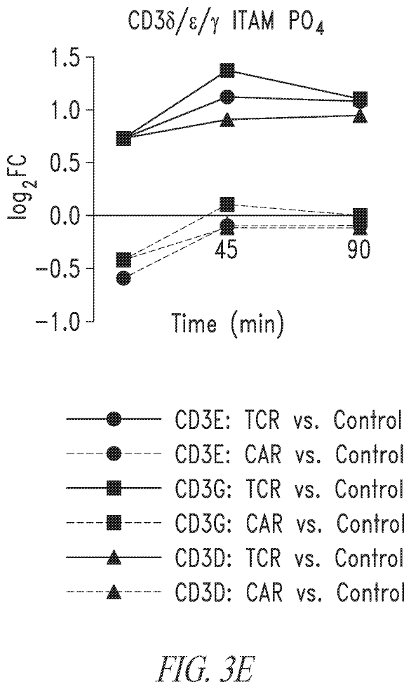

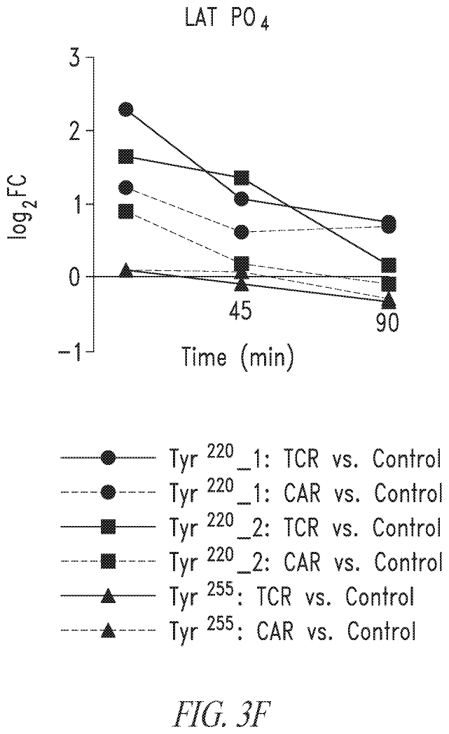

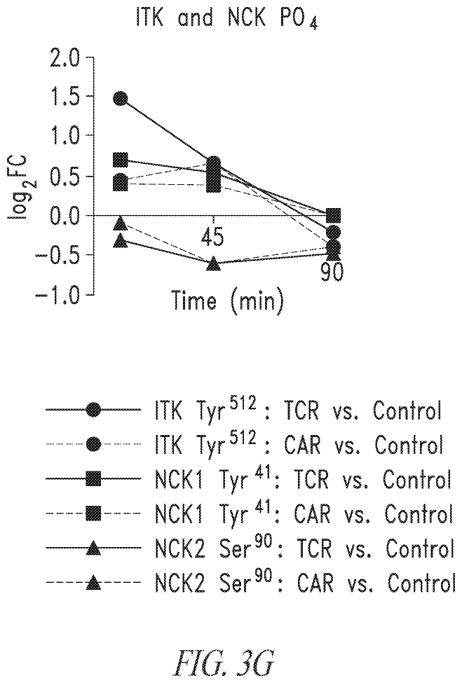

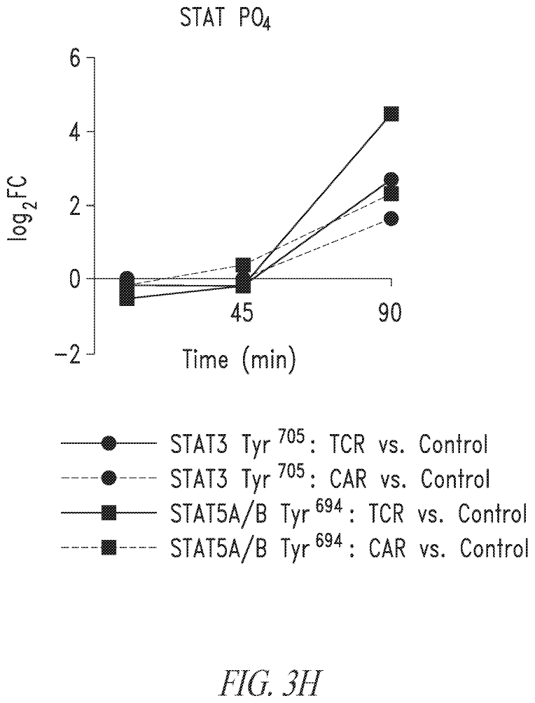

Functional and signaling data suggested that TCR stimulation more efficiently converts initial receptor PO₄ into downstream signals that give rise to more potent effector functions. To investigate further, changes in protein PO₄ after CAR or TCR stimulation were directly compared. At the earliest (10-minute) stimulation time point, hundreds of PO₄ sites were more intensely modulated by TCR than CAR stimulation (FIG. 3A). Signaling intermediates (AHNAK, CBLB, IKBKB, INPP4A, RAPGEF1, RAPGEF2 and SHC1) as well as RNA binding proteins (DDX17, EIF6, EIF4B, and RBM25) were also phosphorylated by TCR stimulation, but not by CAR stimulation (FIG. 3D). After 45 minutes and 90 minutes, a strong correlation between PO₄ site changes in TCR and CAR stimulated samples was observed. Only 30 sites at the 45-minute time point possessed fold change values that differed by more than 2-fold between TCR and CAR treated samples (FIG. 3B). Among these, CD3δ, CD3ε, CD3γ, LAT, LAX1 and PAG1 were all more intensely phosphorylated by TCR than CAR stimulation. After 90 minutes of stimulation, 41 sites possessed fold change values that differed by more than 2-fold between TCR and CAR treated samples (FIG. 3C). CD3ε and CD3γ remained more intensely phosphorylated by TCR and CAR stimulation, and preferential PO₄ of TAGAP, SOS1, STAT3, STAT5A/B, and NR4A1 was detected after TCR, but not CAR, stimulation. Plotting individual sites across time further demonstrated that signaling adaptors known to be critical for TCR signaling were less intensely phosphorylated by CAR stimulation than TCR stimulation (FIGS. 3E-3H). These differences were consistent with the weaker SLP-76 and PLC-γ1 PO₄ measured after CAR stimulation by Western blot (FIG. 2M), and indicated that CARs may not recapitulate all TCR-induced signaling events.

### Example 2

### Design and Testing of Cars Containing CD3ε Domains

### CD3ε Sequences Improve CAR T Cell Recognition of ROR1^{lo} Tumor Cells

The relatively weaker PO₄ of key TCR signaling adaptors after CAR stimulation may be responsible for increased antigen density requirements. To determine whether CARs could more efficiently engage LAT, ITK and NCK1/2, additional ITAMs and/or protein recruitment domains were considered for inclusion in the CAR backbone. The CD3ε endodomain was chosen for incorporation into an existing 4-1BB/CD3ζ CAR backbone because cross-linking endogenous CD3ε is sufficient for T cell activation and 4-1BB/CD3ζ CAR signaling appears less likely to promote T cell exhaustion in vivo than CD28/CD3ζ CAR signaling (Salter et al., Sci. Signal. 11:eaat6753 (2018); Cherkassky et al., J. Clin. Invest. 126:3130-3144 (2016)). The CD3ε endodomain includes three known signaling motifs. A basic residue-rich sequence (BRS) lies adjacent the membrane at the N-terminal portion of the endodomain and is known to bind Lck (see Li et al., PNAS U.S.A. 114:E5891-5899 (2017)). A proline-rich sequence (PRS) resides further within the endodomain, and may enhance TCR sensitivity to certain antigens by interacting with Nck1 (see Tailor et al., J Immunol. 181:243-255 (2008); Mingueneau et al., Nat. Immunol. 9:522-532 (2008)). An ITAM is positioned furthest from the membrane and PO₄ of its tyrosine residues is believed to activate ZAP-70 (see Brownlie & Zamoyska, Nat. Rev. Immunol. 13:257-269 (2013)). Prior experiments indicated that additional Lck increases CAR signal strength (data not shown). To provide additional capacities without increased CAR-Lck association due to the CD3ε BRS, a CAR variant containing only the CD3ε PRS and ITAM was also designed (“CD3ε PRS_ITAM”).

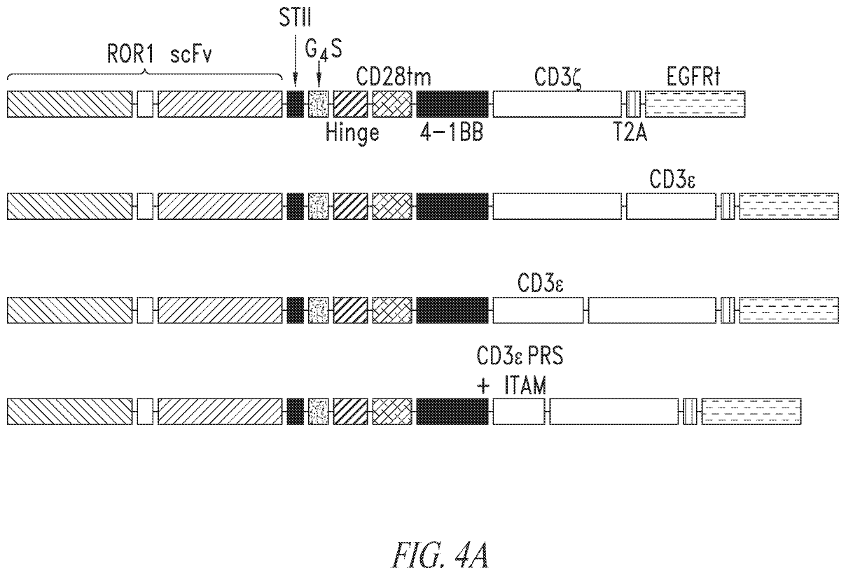

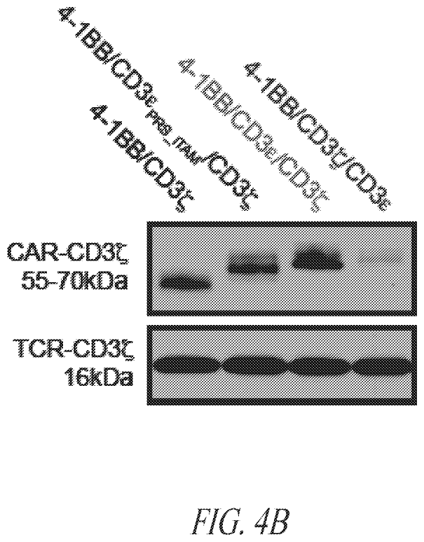

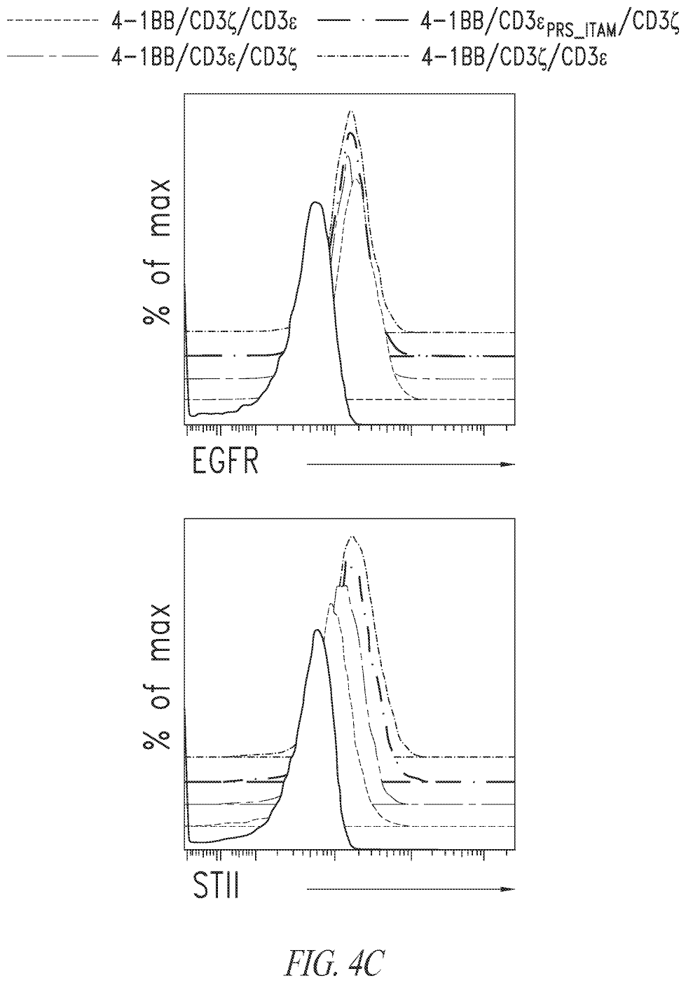

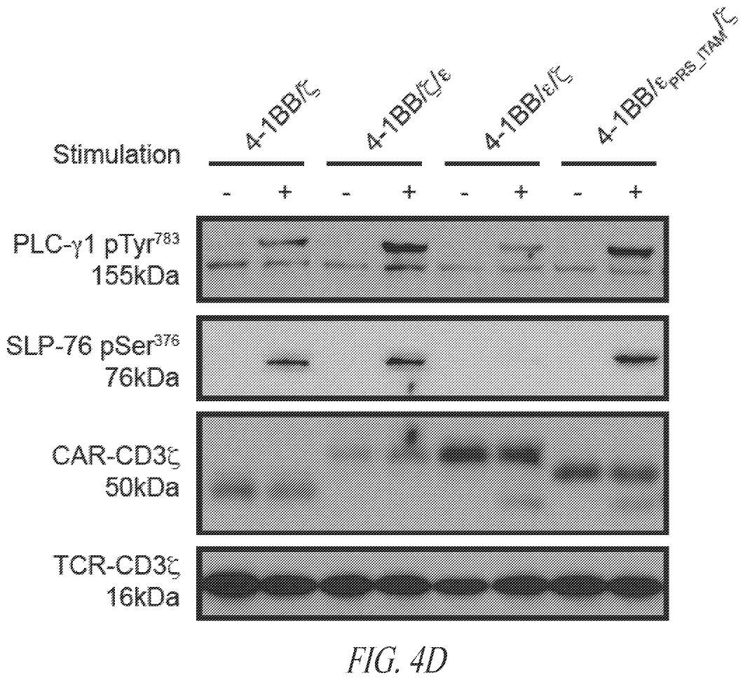

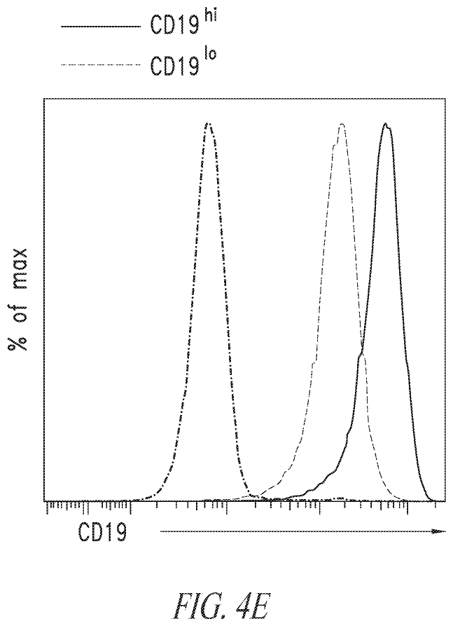

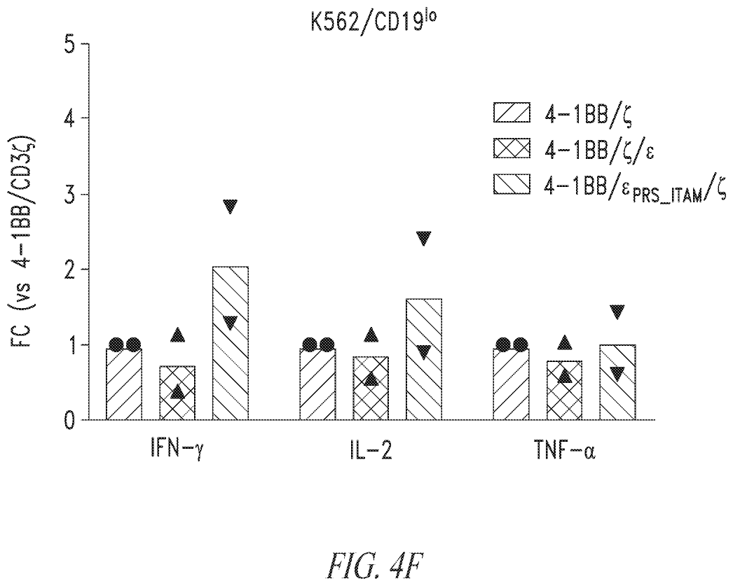

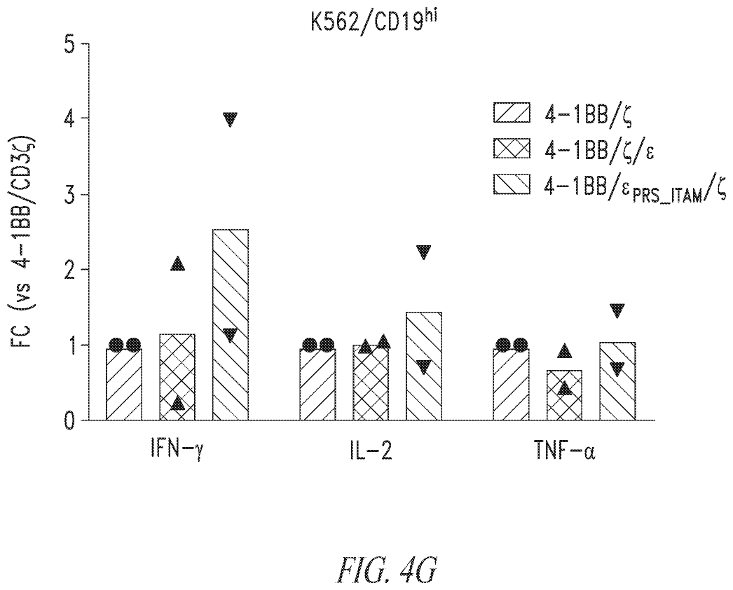

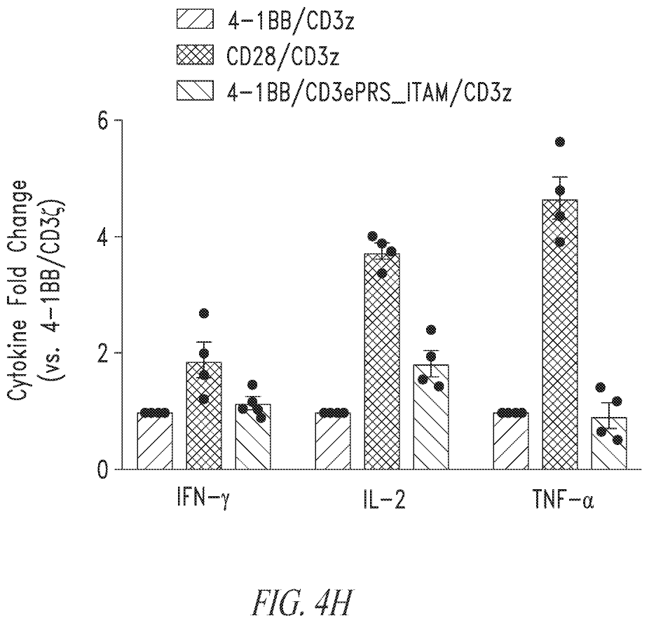

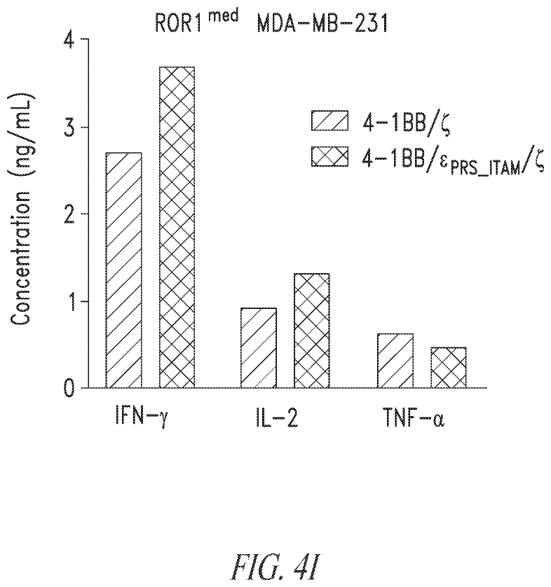

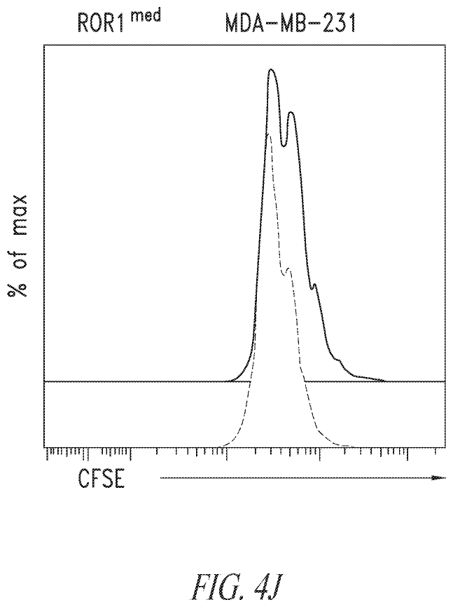

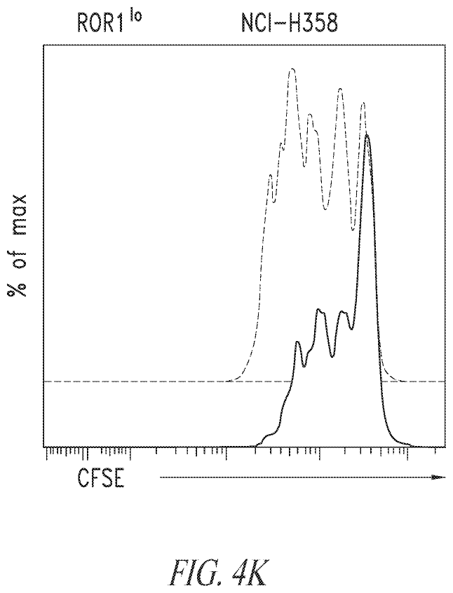

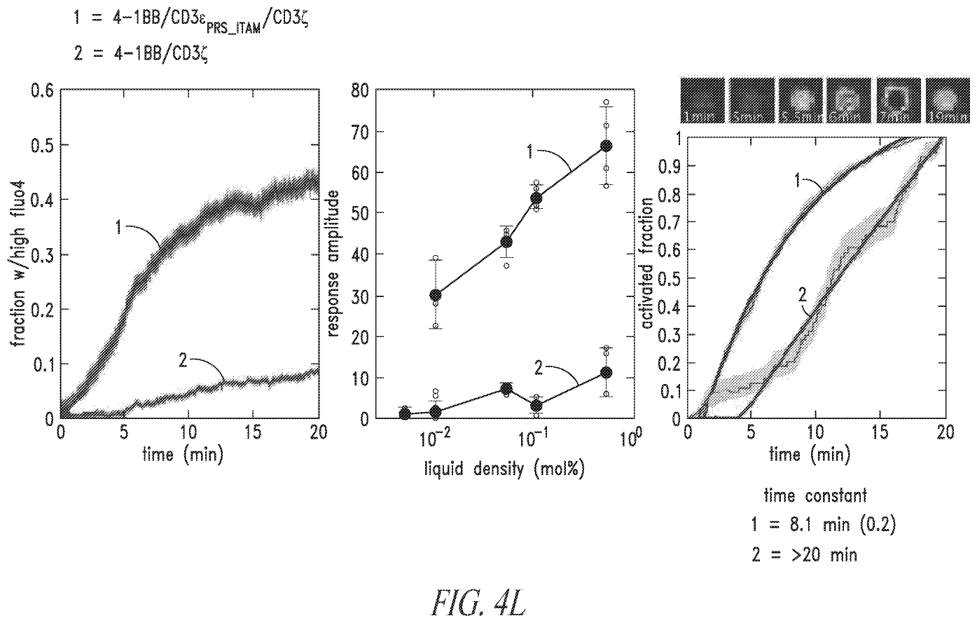

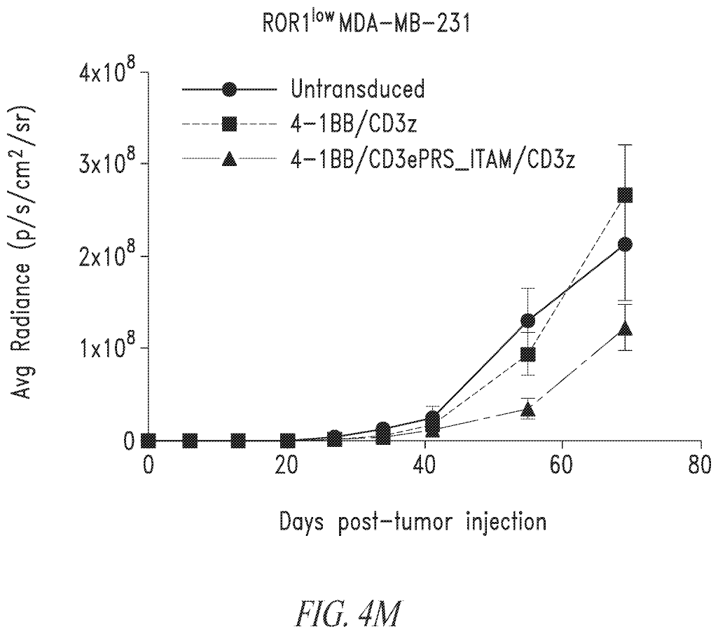

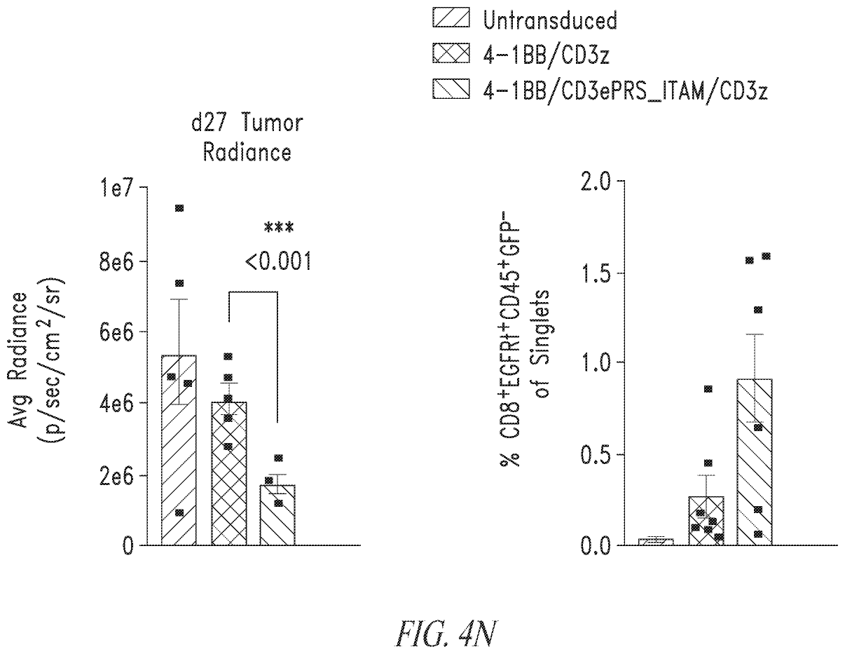

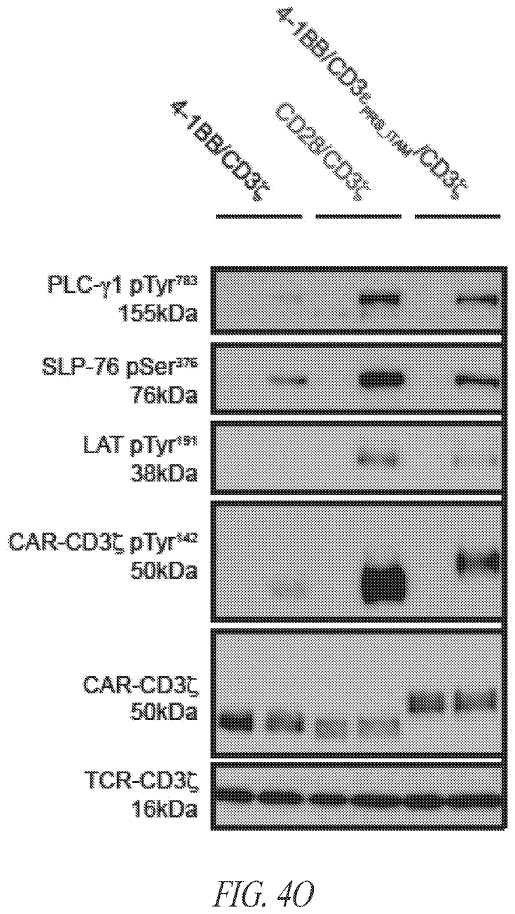

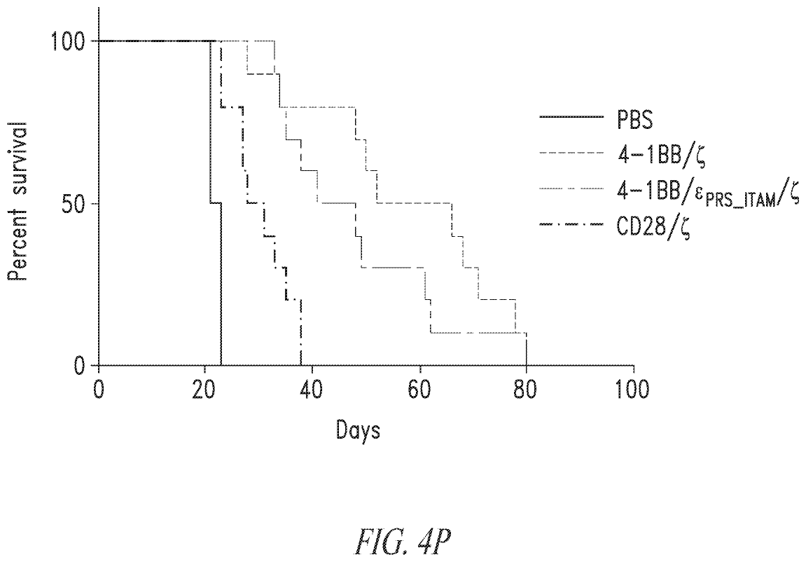

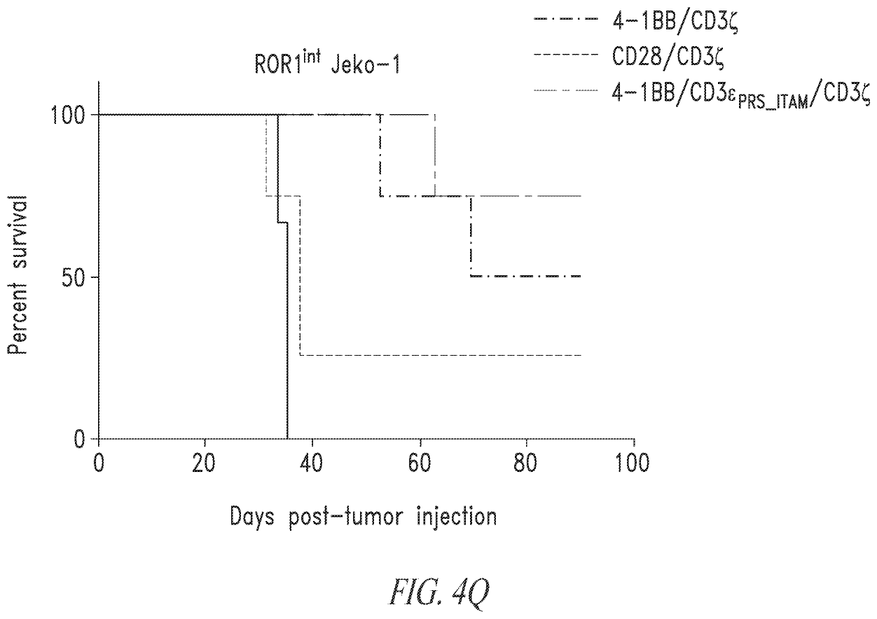

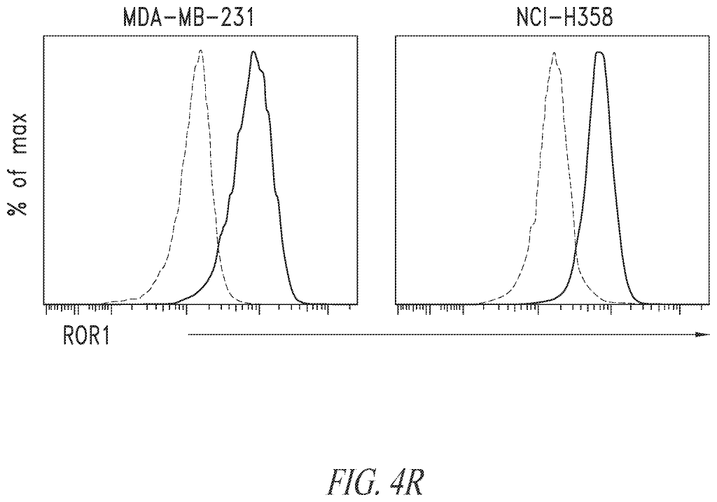

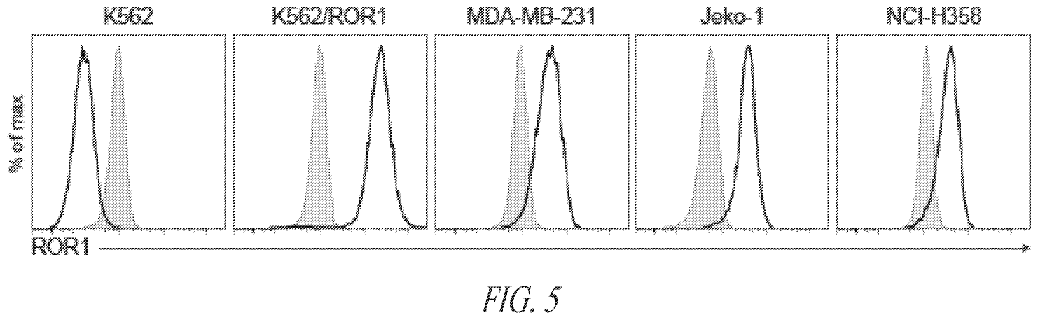

All or a portion of the CD3ε endodomain was placed either before or after the CD3ζ domain in CD19-specific or ROR1-specific 4-1BB/CD3ζ CARs (FIG. 4A). T cells expressing these constructs were examined to determine whether the CAR could augment recognition of low-density antigens. CARs contained an STII sequence to allow measurement of cell surface expression. The addition of full-length CD38 either before or after CD3ζ resulted in lower CAR surface expression, however the CAR containing only the CD38 PRS and ITAM domains was expressed at similar levels to a 4-1BB/CD3ζ CAR on the cell surface (FIGS. 4B and 4C). CD19-specific CARs including the full length endodomain of CD3ε or the PRS_ITAM domain of CD3ε efficiently activated T cells after STII microbead crosslinking, although placing the entire CD3ε endodomain between the 4-1BB and CD3ζ domains surprisingly led to relatively weaker SLP-76 Ser³⁷⁶ and PLC-γ1 Tyr⁷⁸³ PO₄ after stimulation (FIG. 4D). CD19-specific CD3ε CAR T cell variants were then co-cultured with K562/CD19 cells that were previously sorted for high or lower CD19 expression (FIG. 4E). The addition of all or a signaling portion of the CD3ε endodomain did not enhance CAR T cell cytokine production at either antigen density (FIGS. 4F and 4G). Furthermore, in vitro stimulation of ROR1-specific CAR T cells with ROR1^{h} K562/ROR1 or ROR1^{med} MDA-MB-231 tumor cell lines demonstrated that 4-1BB/CD38_{PRS_ITAM}/CD3ζ CAR T cells secreted similar levels of pro-inflammatory cytokines to 4-1BB/CD3ζ CAR T cells, and these cytokine levels were markedly less than those secreted by CD28/CD3ζ CAR T cells (FIGS. 4H, 4I and 5). Despite similar cytokine production, 4-1BB/CD3εPRS_ITAM/CD3ζ CAR T cells proliferated to a greater extent upon co-culture with ROR1^{int} MDA-MB-231 and ROR1^{lo} NCI-H358 cells (FIGS. 4J and 4K).

Antigen sensitivity of the highly expressed 4-1BB/CD38_{PRS_ITAM}/CD3ζ was compared to a conventional 4-1BB/CD3ζ CAR. Fluorescence microscopy measurements of Ca²⁺ mobilization using Fluo-4 AM showed clear differences between CAR constructs in the kinetics and fraction of activated CAR T cells within minutes of antigen encounter. At low antigen density, fewer than 10% of 4-1BB/CD3ζ CAR T cells were activated during 20 minutes of exposure to the antigen-containing bilayer. In contrast, significantly a greater fraction of 4-1BB/CD38_{PRS_ITAM}/CD3ζ CAR T cells was triggered (FIG. 4L). The enhanced Ca²⁺ mobilization responses of 4-1BB/CD38_{PRS_ITAM}/CD3ζ compared to 4-1BB/CD3ζ persisted through a range of ROR1 densities. These results indicate that CAR antigen sensitivity can be augmented by without marked increases in pro-inflammatory cytokine production, which has been associated with a CD28 costimulatory domain and can lead severe cytokine release syndrome (Maloney, Nat. Rev. Clin. Oncol. 2018 15:4 16, 279-280 (2019)).

### CD3ε Sequences Enhance CAR Antitumor Function in Antigen^{low} Settings

Consistent with improved antigen sensitivity, 4-1BB/CD38_{PRS_ITAM}/CD3ζ CAR T cells displayed improved in vivo antitumor function in a xenograft tumor model of ROR1^{int} MDA-MB-231 breast adenocarcinoma (FIG. 4M, FIG. 5). Infusion of a sub-curative dose of CD8⁺ CAR T cells into tumor-bearing mice demonstrated that 4-1BB/CD3ε_{PRS_ITAM}/CD3ζ CAR T cells better controlled tumor at both early and late time points compared to 4-1BB/CD3ζ CAR T cells (FIGS. 4M, 4N (left)). The improved tumor control occurred despite similar intratumor CAR T cell frequencies that did not meet statistical significance (FIG. 4N (right)).

Demonstrating that 4-1BB/CD3ζ CARs containing CD3ε sequences possessed higher antigen sensitivity and antitumor function posed the question of whether the additional signaling domains made the CAR T cells signal and behave more like T cells expressing a conventional CD28/CD3ζ CAR (Hamieh et al., Nature 545:423 (2019); Drent et al., Clin. Cancer Res. 25:4014-4025 (2019)). To this end, CAR signaling was studied by Western blot. 10 minutes of 4-1BB/CD38_{PRS_ITAM}/CD3ζ stimulation promoted increased PO₄ of LAT Tyr¹⁹¹, SLP-76 Ser³⁷⁶, or PLC-γ1 Tyr⁷⁸³ PO₄ relative to 4-1BB/CD3ζ CAR T cells (FIG. 4O). Notably, CD3ζ Tyr¹⁴² was not phosphorylated as strongly after stimulation of the novel CAR as with CD28/CD3ζ CARs, but yielded similar levels of LAT and PLC-γ1 PO₄. Thus, the novel CARs, informed by the results comparing signaling with TCRs, more efficiently convert CAR-CD3ζ PO₄ into downstream PO₄ of signaling intermediates.

It has previously been shown that CD28/CD3ζ CAR T cells are less effective than 4-1BB/CD3ζ CAR T cells at low doses for treating lymphoma and leukemia xenografts in NSG mice due to the rapid development of CD28/CD3ζ CAR T cell exhaustion (see Salter et al. (supra); Cherkassky et al. (supra); Feucht et al., Nat. Med. 545:423 (2018)). The antitumor function of 4-1BB/CD38PRS_ITAM/CD3ζ CAR T cells was therefore compared to conventional CD28/CD3ζ and 4-1BB/CD3ζ CAR T cells in a second tumor model of ROR1^{int} mantle cell lymphoma. In this model, treatment with CD28/CD3ζ CAR T cells resulted in rapid tumor outgrowth. In contrast, adoptive transfer of 4-1BB/CD3ζ and 4-1BB/CD3ε_{PRS_ITAM}/CD3ζ CAR T cells resulted in sustained antitumor function and improved survival (FIG. 4Q). To further examine the antitumor capabilities of 4-1BB/CD38PRS_ITAM/CD3ζ CAR T cells, CAR T cells were injected into NSG mice bearing disseminated CD19⁺ Raji lymphoma. The 4-1BB/CD38_{PRS_ITAM}/CD3ζ CAR design slightly reduced antitumor function and median survival compared to mice treated with conventional 4-1BB/CD3ζ CAR T cells, but still outperformed CD28/CD3ζ CAR T cells (FIG. 4P). Thus, the signals provided by 4-1BB/CD38_{PRS_ITAM}/CD3ζ CARs differed qualitatively from those provided by CD28/CD3ζ CARs.

### Example 3

### Materials and Methods

Acquisition of Peripheral Blood T Cells from Healthy Donors

Healthy adults (>18 years-old) were enrolled in Institutional Review Board-approved studies for peripheral blood collection or leukapheresis. Informed consent was obtained from all enrollees. Researchers were provided only donor age and a nondescript donor ID number and blinded to all personally identifiable information about study participants. CD4⁺ and CD8⁺ T cells were isolated using the EasySep Human CD4⁺ and CD8⁺ T Cell Isolation Kits (StemCell Technologies). For the three shotgun mass spectrometry experiments, CD8+CD45RO+ T cells were enriched by EasySep Human Memory CD8+ T Cell Enrichment Kit (StemCell Technologies). Isolations were performed in accordance with manufacturer's instructions.

### Cell Culture

LentiX cells (Clontech) were cultured in DMEM (Gibco) supplemented with 10% fetal bovine serum, 1 mM L-glutamine (Gibco), 25 mM HEPES (Gibco), and 100 U/mL penicillin/streptomycin (Gibco). K562 (CCL-243), NCI-H358 (CRL-5807), MDA-MB-231 (HTB-26), and Raji (CCL-86) cells were obtained from American Type Culture Collection and cultured in RPMI-1640 (Gibco) supplemented with 5% fetal bovine serum, 1 mM L-glutamine, 25 mM HEPES, and 100 U/mL penicillin/streptomycin. The generation of K562/CD19 and K562/ROR1 cells was previously described (Salter:2018 ft). Primary human T cells were cultured in CTL medium consisting of RPMI-1640 supplemented with 10% human serum, 2 mM L-glutamine, 25 mM HEPES, 100 U/mL penicillin/streptomycin, 50 μM β-mercaptoethanol (Sigma), and 50 U/mL IL-2 (Prometheus). All cells were cultured at 37° C. and 5% CO2, and tested bi-monthly for the absence of mycoplasma using MycoAlert Mycoplasma Detection Kit (Lonza).

### Generation of Chimeric Antigen Receptors (CARs) and Recombinant Lentiviral Vectors

CD19-specific and ROR1-specific 4-1BB/CD3ζ and CD28/CD3ζ CAR constructs with an R12 scFv and IgG4 hinge have been previously described (Hudecek:2013ek; Sommermeyer:2016ky). CD3ε CARs were constructed by inserting a single Strep-tag II sequence and two G4S linkers between the FMC63 or R12 single chain variable fragment and IgG4 hinge (Liu:2016hy). These were linked to the 27-amino acid transmembrane domain of human CD28 (UniProt: P10747), a signaling module comprising the 42 amino acid cytoplasmic domain of human 4-1BB (UniProt: Q07011), and the 112-amino acid cytoplasmic domain of isoform 3 of human CD3ζ (UniProt: P20963-3). The entire CD3ε endodomain (UniProt: PO₇₇₆₆) or a truncated endodomain (amino acids 179-207) containing PRS and ITAM components was inserted either immediately before or after the CD3ζ endodomain. All CAR constructs were linked by T2A sequence to a truncated CD19 (CD19t) or epidermal growth factor receptor (EGFRt), codon-optimized, and cloned into a HIV7 lentiviral vector. All cloning was performed by PCR, enzyme digest, and/or Gibson assembly. Plasmids were verified by capillary sequencing and restriction digest.

### Lentivirus Preparation and Transduction

Lentivirus was generated by transient transfection of LentiX cells using psPAX2, pMD2.G, and a CAR encoding lentiviral vector. Primary human T cells were activated and transduced as described in Salter et al. (supra). To prepare K562 cells expressing HLA-B8, LentiX cells were transiently transfected with psPAX2, pMD2.G, and a HIV7 lentiviral vector encoding HLA-B8. To prepare K562/B8 cells expressing EBV antigens or ROR1, LentiX cells were transiently transfected with MLV g/p, 10A1, and a mp71 retroviral vector encoding GFP and an EBV peptide minigene, or human ROR1. The mp71 retroviral vector was a gift of Dr. Wolfgang Uckert, Max Delbruck Center for Molecular Medicine. To prepare Raji/ffluc cells, LentiX cells were transiently transfected with psPAX2, pMD2.G, and an HIV7 lentiviral vector encoding GFP and firefly luciferase. Two days later, viral supernatant was filtered using a 0.45 μm PES syringe filter and added to K562 or Raji cells. Five days later, transduced cells were stained monoclonal antibodies specific for HLA-B8 (REA145, Miltenyi Biotec) and ROR1 (2A2, Miltenyi Biotec) and FACS-sorted on a FACSAria II to greater than 97% purity.

### T Cell Expansion for Mass Spectrometric and Functional Analyses

FACS-sorted CD8+tetramer+CD19t+ or CD8+EGFRt+ cells were expanded over a single stimulation cycle prior to MS and/or functional analyses. Bi-specific and ROR1-specific CAR T cells were expanded using a rapid expansion protocol containing purified OKT3, irradiated LCL, and irradiated PBMC, and assayed 11 days after stimulation. CD19-specific CAR T cells were expanded by co-culture with irradiated CD19+ lymphoblastoid cell lines (LCL) in a 1:7 (T cell:LCL) ratio and assayed 8 days after stimulation. During expansion, cultures were fed with fresh CTL media containing 50 IU/mL IL-2 every 2-3 days.

### Flow Cytometry and Cell Phenotyping

T cells were stained with a 1:100 dilution of fluorophore-conjugated monoclonal antibodies specific for human CD4 (RPA-T4), CD8 (SK1), CD19 (HIB19), CD28 (CD28.2), CD45 (HI30), CD45RO (UCHL1), CD62L (DREG56) or CD279 (eBioJ105) purchased from BD Biosciences, ThermoFisher, or Biolegend. T cells were also stained with isotype control fluorophore-conjugated antibodies when appropriate. Cetuximab (anti-EGFR, Bristol Myers Squibb) was biotinylated using the EZ-Link Sulfo-NHS-Biotin kit (ThermoFisher) followed by cleanup with a Zeba Spin Desalting Column (ThermoFisher) and used to stain T cells in conjunction with Streptavidin-APC (ThermoFisher). DNA content staining was performed by fixing T cells with 70% ice-cold ethanol, permeabilizing cells with 1% Triton-X (Sigma), degrading RNA with 100□g/mL RNAse A (ThermoFisher), and staining DNA with 20 μg/mL Propidium Iodide (ThermoFisher). All data was collected on a FACSCanto II or FACSAria II (BD Biosciences).

### SCT, SCT/CD28, ROR1, STII and Control Microbead Preparation

Recombinant single chain trimer (SCT) and human ROR1 were produced at the Fred Hutchinson Cancer Research Center. 1 mL Streptavidin-Coated Magnetic Particles (Spherotech) was washed once in excess lx PBS supplemented with 100 U/mL penicillin/streptomycin (PBS+P/S) using a benchtop magnet. SCT and ROR1 microbeads were prepared by resuspending beads in 1 mL PBS+P/S and then slowly adding recombinant protein while vortexing the solution. Protein was added at the indicated molar ratios according to manufacturer's predetermined molar binding capacities. SCT/CD28 microbeads were prepared by resuspending beads in 1 mL PBS+P/S and then slowly adding recombinant SCT protein and biotinylated CD28 mAb (CD28.2, ThermoFisher) at a 3:1 molar ratio. STII microbeads were prepared by resuspending beads in 1 mL PBS+P/S and then slowly adding 16.67 g anti-STII biotin mAb (GenScript) while vortexing the bead solution. All microbeads were incubated overnight at 4° C. on a 3D orbital shaker, washed three times with excess PBS+P/S using a benchtop magnet, and resuspended in 4 mL PBS+P/S for STII microbeads or 1 mL PBS+P/S for control, SCT, SCT/CD28 and ROR1 beads. To make control beads, 1 mL Streptavidin-Coated Magnetic Particles was washed once using a benchtop magnet and the bead pellet was resuspended in 1 mL PBS+P/S. All beads were stored at 4° C.

### Cell Stimulations, Protein Lysates, RNA Isolation

CAR T cells were washed and resuspended in warm CTL medium. T cells were brought to a concentration of 2×10⁷ cells per mL and incubated with control, SCT, SCT/CD28, ROR1, or STII microbeads in a 37° C. water bath. After the allotted time, cells were quickly washed twice using ice-cold PBS, then lysed in a 6M Urea, 25 mM Tris (pH 8.0), 1 mM EDTA, 1 mM EGTA solution supplemented with protease (Sigma) and phosphatase inhibitors (Sigma) at a 1:100 dilution, hereon referred to as lysis buffer. Lysates were sonicated for 15 seconds prior to centrifuging at 10,000×g and 4° C. for 10 minutes. Beads were removed during lysate clearing.

Protein Digestion, TMT Labeling, and Phosphotyrosine (pTyr) Peptide Immunoprecipitation

Protein was quantified in lysates by Micro BCA Assay (ThermoFisher), and lysates were diluted to 2 mg/mL using lysis buffer. Lysates were reduced in 24 mM TCEP (ThermoFisher) for 30 minutes at 37° C. with shaking, followed by alkylation with 48 mM iodoacetamide (Sigma) in the dark at room temperature for 30 minutes. Lysates were then diluted with 200 mM Tris (pH 8.0), to a urea concentration of 2M. Lys-C (Wako) was dissolved in 25 mM Tris (pH 8.0) at 200 ug/mL and added to lysates at 1:100 (enzyme:protein) ratio by mass and incubated for 2 hours at 37° C. with shaking. Samples were further diluted with 200 mM Tris (pH 8.0) to a urea concentration of 1M before adding trypsin at a 1:50 trypsin:protein ratio. After 2 hours, a second trypsin aliquot was added at a 1:100 trypsin:protein ratio. Digestion was carried out overnight at 37° C. with shaking. After 16 hours, the reaction was quenched with formic acid to a final concentration 1% by volume. Samples were desalted using Oasis HLB 96-well plates (Waters) and a positive pressure manifold (Waters). The plate wells were washed with 3×400 μL of 50% MeCN/0.1% FA, and then equilibrated with 4×400 μL of 0.1% FA. The digests were applied to the wells, then washed with 4×400 μL 0.1% FA before being eluted drop by drop with 3×400 μL of 50% MeCN/0.1% FA. The eluates were lyophilized, followed by storage at −80° C. until use. For TMT labeling (ThermoFisher), desalted peptides were resuspended in 50 mM HEPES at 1 mg/mL based on starting protein mass. TMT reagents were resuspended in 257 μL MeCN and transferred to the peptide sample. Samples were incubated at room temperature for 1 hour with mixing. Labeling reactions were quenched by the addition of 50 μL of 5% hydroxyl Amine (Sigma) and incubated for 15 minutes at room temperature with mixing. The independent labeling reactions were then pooled together and lyophilized. The labeled peptides were desalted as above and then lyophilized and stored at −80° C. Immunoprecipitation of pTyr peptides was performed using the PTMScan P-Tyr-1000 Kit (Cell Signaling). The enriched pTyr peptide fraction was purified using a C18 Spin Tip (ThermoFisher), lyophilized, and stored at −80° C. until analysis. The flow-through fraction was desalted, lyophilized, and stored at −80° C.

### Basic (High pH) Reverse Phase Liquid Chromatography

The desalted and pTyr peptide-depleted flow-through was fractionated by high-pH reverse phase (RP) liquid chromatography. 4 mg of the protein digest was loaded onto a LC system consisting of an Agilent 1200 HPLC with mobile phases of 5 mM NH4HCO3 (pH 10) (A) and 5 mM NH4HCO3 in 90% MeCN (pH 10) (B). The peptides were separated by a 4.6 mm×250 mm Zorbax Extend-C18, 3.5 μm, column (Agilent) over 96 minutes at a flow rate of 1.0 mL/min by the following timetable: hold 0% B for 9 minutes, gradient from 0 to 10% B for 4 minutes, 10 to 28.5% B for 50 minutes, 28.5 to 34% B for 5.5 minutes, 34 to 60% B for 13 minutes, hold at 60% B for 8.5 minutes, 60 to 0% B for 1 minute, re-equilibrate at 0% B for 5 minutes. 1 minute fractions were collected from 0-96 minutes by the shortest path by row in a 1 mL deep well plate (ThermoFisher). The high pH RP fractions were concatenated into 24 samples by every other plate column starting at minute 15 (e.g., sample 1 contained fractions from wells B10, D10, F10, etc.). The remaining fractions were combined such that fractions from 12 to 14 minutes were added to sample 1, all fractions after 86 minutes were added to sample 24, and all fractions from 0 to 11 minutes were combined into sample ‘A’. 95% of every 12th fraction of the 24 samples was combined (1,13; 2,14; . . . ) to generate 12 more samples, which were dried down and stored at −80° C. prior to phosphopeptide enrichment by immobilized metal affinity chromatography.

### Immobilized Metal Affinity Chromatography (IMAC)

IMAC enrichment was performed using Ni-NTA-agarose beads (Qiagen) stripped with EDTA and incubated in a 10 mM FeCl3 solution to prepare Fe3+-NTA-agarose beads. Fractionated lysate was reconstituted in 200 μL of 0.1% TFA in 80% MeCN and incubated for 30 minutes with 100 μL of the 5% bead suspension while mixing at room temperature. After incubation, beads were washed 3 times with 300 μL of 0.1% TFA in 80% MeCN. Phosphorylated peptides were eluted from the beads using 200 μL of 70% ACN, 1% Ammonium Hydroxide for 1 minute with agitation at room temperature. Samples were transferred into a fresh tube containing 60 μL of 10% FA, dried down and re-suspended in 0.1% FA, 3% MeCN. Samples were frozen at −80° C. until analysis.

### Nano-Liquid Chromatography-Tandem Mass Spectrometry

Phosphopeptide-enriched samples were analyzed by LC-MS/MS on an Easy-nLC 1000 (ThermoFisher) coupled to an LTQ-Orbitrap Fusion mass spectrometer (ThermoFisher) operated in positive ion mode. The LC system, configured in a vented format consisted of a fused-silica nanospray needle (PicoTip emitter, 50 μm ID×20 cm, New Objective) packed in-house with ReproSil-Pur C18-AQ, 3 μm and a trap (IntegraFrit Capillary, 100 μm ID×2 cm, New Objective) containing the same resin as in the analytical column with mobile phases of 0.1% FA in water (A) and 0.1% FA in MeCN (B). The peptide sample was diluted in 20 μL of 0.1% FA, 3% MeCN, and 8.5 μL was loaded onto the column and separated over 210 minutes at a flow rate of 300 nL/min with a gradient from 5 to 7% B for 2 minutes, 7 to 35% B for 150 minutes, 35 to 50% B for 1 minute, hold 50% B for 9 minutes, 50 to 95% B for 2 minutes, hold 95% B for 7 minutes, 95 to 5% B for 1 minute, re-equilibrate at 5% B for 38 minutes. A spray voltage of 2000 V was applied to the nanospray tip. MS/MS analysis occurred over a 3 second cycle time consisting of 1 full scan MS from 350-1500 m/z at resolution 120,000 followed by data dependent MS/MS scans using HCD activation with 27% normalized collision energy of the most abundant ions. Selected ions were dynamically excluded for 45 seconds after a repeat count of 1.

### Western Blotting

Equal masses of protein lysate were loaded into 4-12% Bis-Tris NuPAGE Gels (ThermoFisher). After protein transfer onto nitrocellulose membranes (ThermoFisher), membranes were blocked with Western Blocking Reagent (Sigma). Membranes were stained with primary and secondary antibodies diluted in SuperBlock (ThermoFisher) supplemented with 0.1% Tween. The following antibodies were used: anti-CD247 (8D3, BD Biosciences), anti-CD247 pTyr¹⁴² (K25-407.69, BD Biosciences), anti-SLP-76 (polyclonal, Cell Signaling), anti-SLP-76 pSer³⁷⁶ (D9D6E, Cell Signaling), anti-PLC-γ1 (D9H10, Cell Signaling), anti-PLC-γ1 pTyr⁷⁸³ (D6M9S, Cell Signaling), anti-mouse horseradish peroxidase (HRP) (polyclonal, Cell Signaling), and anti-rabbit HRP (polyclonal, Cell Signaling). Typical antibody dilutions ranged from 1:10,000 to 1:2500.

### In Vitro Functional Assays

CAR T cells were co-cultured with tumor cells at a T cell to tumor cell ratio of 2:1 or beads at a ratio of 7.5 μL beads per 1×10⁶ cells. Intracellular cytokine staining was performed by incubating T cells with tumor cells or beads and GolgiStop (BD Biosciences) for 5 hours. Cells were fixed and permeabilized using FoxP3/Transcription Factor Staining Buffers (ThermoFisher) and stained with fluorochrome-labeled monoclonal antibodies specific for IFN-γ (B27), IL-2 (MQ1-17H12), and TNF-α (Mab11) purchased from Biolegend and BD Biosciences. Cytokine concentrations in cellular supernatant were quantified by ELISA (ThermoFisher) 24 hours after stimulation. T cell proliferation was quantified by staining CAR T cells with a 0.2 μM solution of carboxyfluorescein succinimidyl ester (CFSE) dye (ThermoFisher) and incubating T cells with tumor cells or beads for 72 hours.

### Transfer of T Cells in NOD SCID/γc−/− (NSG) Mice

Six- to eight-week-old male or female NSG mice were obtained from the Jackson Laboratory or bred in-house. Mice were engrafted via tail vein with 5×10⁵ Raji/ffluc, MDA-MB-231/ffluc, or Jeko/ffluc cells and, 7 days later, injected intravenously with a defined product of CAR T cells. For Raji experiments, CD8⁺ and CD4⁺ CAR T cells were mixed together in a 1:1 ratio. For MDA-MB-231 and Jeko experiments, CD8⁺ T cells were infused. Bioluminescence imaging was performed as described and mice were followed for survival. Mice handlers were blinded to group allocation. The Fred Hutchinson Cancer Research Center Institutional Animal Care and Use Committee approved all experimental procedures.

### Shotgun Mass Spectrometry Data Analysis

Raw MS/MS spectra from each replicate experiment were searched together against the reviewed Human Universal Protein Resource (UniProt) sequence database (release 2016_01) with common laboratory contaminants using the MaxQuant/Andromeda search engine version 1.6.0.1 (Cox:2008ir). The search was performed with a tryptic enzyme constraint for up to two missed cleavages. Variable modifications were oxidized methionine, phosphorylated serine, phosphorylated threonine, and phosphorylated tyrosine. Carbamidomethylated cysteine was set as a static modification. Peptide MH+ mass tolerances were set at 20 ppm. The overall FDR was set at ≤1% using a reverse database target decoy approach.

For the three TMT experiments, phosphopeptide site localization was determined by MaxQuant and converted to phosphorylation sites using Perseus version 1.6.0.7 (Tyanova:2016ds). At this step, reverse hits and potential contaminants were excluded from further analysis. Data normalization was performed by scaling each TMT channel to the channel median, followed by a log 2transformation. Stimulation vs. control ratios were calculated by subtracting the appropriate control channels from stimulated channels. Due to incomplete MS sampling, some phosphorylation sites (features) were only found in one or two replicate experiments, and a much smaller minority (<1%) of sites were not found in every TMT channel.

Differential expression analyses over PO4 sites were performed using the limma statistical framework and associated R package (Ritchie:2015fa, Smyth:2004gh). For these analyses, only those features were kept that had values in at least two experiments and all TMT channels, leaving 19,608 quantified PO4 sites. A linear model was fitted to each PO4 site, and empirical Bayes moderated t-statistics were used to assess differences in expression/abundance. Contrasts comparing stimulation vs control treatments were tested. Intraclass correlations were estimated using the duplicate Correlation function of the limma package to account for measures originating from the same patients and the same antigens (Smyth:2005iy). An absolute log 2fold change cutoff (stimulation versus control) of 1 and a false discovery rate (FDR) cutoff of 5% were used to determine differentially expressed PO₄ sites. Analyses of signaling networks and KEGG Pathways were performed using StringDB.

### Analysis of T Cell Phenotype, Function, and In Vivo Experiments

FlowJo version 9 (Treestar) was used to analyze flow cytometry files. Prism version 7 (GraphPad Software) was used to plot data and calculate statistics. P values meeting an α=0.05 level of significance are indicated in the figures. The precise statistical tests used are indicated in the figure legends.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/735,702, filed Sep. 24, 2018, and U.S. Provisional Patent Application No. 62/901,194, filed Sep. 16, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein comprising:
(a) an extracellular component comprising a binding domain that specifically binds to an antigen;
(b) a transmembrane domain; and
(c) an intracellular component comprising an effector domain or a functional portion thereof, wherein the effector domain or functional portion or variant thereof comprises:
(i) an Intracellular Tyrosine-based Activation Motif (ITAM) from CD3ε, or a functional portion or variant thereof,
(ii) an ITAM from CD3γ, or a functional portion or variant thereof,
(iii) an ITAM from CD3δ, or a functional portion or variant thereof;
(iv) a Proline Rich Sequence (PRS) from CD3ε, or a functional portion or variant thereof;
(v) a Basic Residue Sequence (BRS) from CD3ε, or a functional portion or variant thereof; or
(vi) any combination of (i)-(v),
wherein
(1) the extracellular component does not comprise a TCR ectodomain; and/or
(2) the fusion protein does not associate with or form a TCR complex when expressed by a T cell.

2. A fusion protein comprising:
(a) an extracellular component comprising a binding domain that specifically binds to an antigen;
(b) a transmembrane domain; and
(c) an intracellular component comprising an effector domain or a functional portion thereof, wherein the effector domain or functional portion thereof comprises:
(i) an Intracellular Tyrosine-based Activation Motif (ITAM) from CD3ε, or a functional variant thereof,
(ii) an ITAM from CD3γ, or a functional variant thereof,
(iii) an ITAM from CD3δ, or a functional variant thereof;
(iv) a Proline Rich Sequence (PRS) from CD3ε, or a functional variant thereof;
(v) a Basic Residue Sequence (BRS) from CD3ε and/or CD3ζ, or a functional variant thereof; or
(vi) any combination of (i)-(v),
wherein the fusion protein does not comprise an ectodomain or a transmembrane domain, or a portion thereof, from CD3ε, CD3δ, and/or CD3γ.

3. The fusion protein of claim 1, wherein the effector domain or functional portion thereof comprises an ITAM from CD3ε, or a functional variant thereof, and a PRS from CD3ε, or a functional variant thereof.

4. The fusion protein of claim 1, wherein the fusion protein does not comprise a BRS from CD3ε, or a functional variant thereof.

5. The fusion protein of claim 1, wherein the effector domain or functional fragment thereof comprises an amino acid sequence having at least 75% identity to the amino acid sequence shown in SEQ ID NO.:8.

6. The fusion protein of claim 1, wherein the effector domain comprises a BRS from CD3ε, or a functional variant thereof.

7. The fusion protein of claim 1, wherein the effector domain comprises an amino acid sequence having at least 75% identity to the amino acid sequence shown in SEQ ID NO.:5 or 111.

8. The fusion protein of claim 1, wherein the effector domain comprises: an ITAM from CD3γ, or a functional variant thereof; and/or comprises an ITAM from CD3δ, or a functional variant thereof.

9. (canceled)

10. The fusion protein of claim 8, wherein the effector domain comprises a BRS from CD3ε, or a functional portion or variant thereof.

11. The fusion protein of claim 1, wherein the effector domain further comprises an ITAM from CD3ζ, or a functional portion or variant thereof.

12. The fusion protein of claim 11, wherein the effector domain comprises two or three of ITAMs (a), (b), and (c) from CD3ζ, or functional portions or variants thereof.

13. (canceled)

14. The fusion protein of claim 1, further comprising a costimulatory domain or a functional portion or variant thereof.

15. The fusion protein of claim 14, wherein the costimulatory domain or functional portion or a variant thereof is from 4-1BB, CD28, OX40, CD27, CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or any combination thereof.

16. (canceled)

17. The fusion protein of claim 14, wherein the intracellular domain comprises the costimulatory domain or functional portion or variant thereof, an endodomain or effector domain from CD3ζ, or a functional portion or variant thereof, and an effector domain from CD3ε, or a functional portion or variant thereof, wherein the effector domain from CD3ε, or functional portion or variant thereof, optionally comprises or consists essentially of a PRS and an ITAM from CD3ε.

18. The fusion protein of claim 17, wherein: (i) the effector domain from CD3ε or functional portion or variant thereof is disposed between the costimulatory domain or functional portion or variant thereof and the endodomain or effector domain from CD3ζ or functional portion or variant thereof; or (ii) the endodomain or effector domain from CD3ζ or functional portion or variant thereof is disposed between the costimulatory domain or functional portion or variant thereof and the effector domain from CD3ε or functional portion or variant thereof.

19-23. (canceled)

24. The fusion protein of claim 1, wherein the binding domain comprises or consists of a scFv, a Fab, a scTCR, or a ligand.

25.-26. (canceled)

27. The fusion protein of claim 1, wherein the binding domain specifically binds to an antigen that is expressed by or is associated with a cancer.

28. The fusion protein of claim 27, wherein the cancer comprises a solid tumor.

29. The fusion protein of claim 1, wherein the antigen is selected from a ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, ERBB2, BRAFV600E, MAGE-A3, MAGE-A4, SSX-2, PRAME, HA-1, CD79a, CD79b, SLAMF7, or CEA antigen.

30.-31. (canceled)

32. The fusion protein of claim 1, wherein the fusion protein, when expressed by a host cell, optionally an immune system cell, provides for or promotes:
(i) improved cell signaling, and/or activity in response to antigen relative to a host cell expressing a reference fusion protein, wherein improved cell signaling optionally comprises increased and/or sustained cytokine production and/or release, and/or phosphorylation of one or more protein associated with an immune cell response to antigen-binding, or the like, such as LAT, PLC-γ1, SLP-76, or any combination thereof,
(ii) improved cell activity in response to antigen relative to a host cell expressing a reference fusion protein, wherein improved cell signaling optionally comprises increased mobilization of intracellular calcium, killing activity, proliferation, earlier activation in response to antigen, or any combination thereof,
(iii) improved cell signaling and/or activity, relative to a host cell expressing a reference fusion protein, upon binding to a target antigen that is expressed at a low level or an intermediate level on a target cell surface;
(iv) reducing or suppressing growth, area, volume, and/or spread of a tumor that expresses an antigen that is recognized and/or specifically bound by the fusion protein, of killing tumor cells, and/or of increasing survival of the subject to a greater degree and/or for a longer period of time as compared to a reference subject administered a host cell expressing a reference fusion protein;
(iv) more efficient phosphorylation of LAT, SLP-76, and/or PLC-γ1 as compared to a reference fusion protein expressed by a host cell;
(v) improved sensitivity to antigen as compared to a host cell expressing a reference fusion protein, but does not produce more, or substantially more, of a pro-inflammatory cytokine as compared to the host cell expressing the reference fusion protein; or
(vi) any combination of (i)-(v).

33. (canceled)

34. The fusion protein of claim 32, wherein:
(i) the reference fusion protein comprises an intracellular domain comprising a 4-1BB costimulatory domain and/or a CD28 costimulatory domain;
(ii) the fusion protein and the reference fusion protein each comprise an endodomain or effector domain from CD3ζ, or a functional portion or variant thereof;
(iii) the host cell expressing the fusion protein and the host cell expressing the reference fusion protein are each a T cell, optionally a CD8+ T cell; or
(iv) any combination of (i)-(iii).

35.-36. (canceled)

37. An isolated polynucleotide encoding the fusion protein of claim 1.

38.-44. (canceled)

45. An expression vector comprising the isolated polynucleotide of claim 37 operably linked to an expression control sequence.

46.-51. (canceled)

52. A host cell comprising the polynucleotide of claim 37.

53. A host cell expressing at its cell surface the fusion protein of claim 1.

54. (canceled)

55. The host cell of claim 52, wherein the host cell is a hematopoietic progenitor cell or a human immune system cell.

56. The host cell of claim 55, wherein the human immune system cell comprises a T cell, a CD4+ T cell, a CD8+ T cell, a CD4− CD8− double negative T cell, a γδ T cell, a natural killer cell, a natural killer T cell, a dendritic cell, or any combination thereof.

57. (canceled)

58. The host cell of claim 56, wherein the T cell is a naïve T cell, a central memory T cell, a stem cell memory T cell, an effector memory T cell, or any combination thereof.

59.-60. (canceled)

61. A composition, comprising a host cell claim 52, and a pharmaceutically acceptable carrier, excipient, or diluent.

62.-63. (canceled)

64. A method of treating a disease or condition in a subject, the method comprising administering to the subject an effective amount of the host cell of claim 53, wherein the disease or condition is characterized by the presence of the antigen.

65. A method of eliciting an immune response against an antigen that is specifically bound by the fusion protein of claim 1, the method comprising administering to a subject comprising or expressing the antigen an effective amount of the host cell of claim 53.

66. The method of claim 64, wherein the disease or condition comprises a hyperproliferative disease or a proliferative disease.

67. The method of claim 64, wherein the disease or condition is a cancer.

68. The method of claim 67, wherein the cancer comprises:
(i) a carcinoma, a sarcoma, a glioma, a lymphoma, a leukemia, a myeloma, or any combination thereof;
(ii) a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof;
(iii) Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optic nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

69.-70. (canceled)

71. The method of claim 67, wherein the cancer comprises a solid tumor.

72. The method of claim 71, wherein the solid tumor is a sarcoma or a carcinoma.

73. The method of claim 72, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)): a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis): an adrenal carcinoma (e.g., Adrenocortical carcinoma), a carcinoma of the testis (e.g., Germ cell carcinoma (Seminoma, Choriocarcinoma, Embryonal carcinoma, Teratocarcinoma), Serous carcinoma): Gastric carcinoma (e.g., Adenocarcinoma): an intestinal carcinoma (e.g., Adenocarcinoma of the duodenum): a colorectal carcinoma; a skin carcinoma (e.g., Basal cell carcinoma, Squamous cell carcinoma); an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, and an adenocarcinoma of the prostate.

74.-83. (canceled)

84. A fusion protein comprising:
(a) an extracellular component comprising a binding domain that specifically binds to an antigen;
(b) a transmembrane domain; and
(c) an intracellular component comprising
(c)(1) a costimulatory domain from 4-1BB, and
(c)(2) an effector domain that comprises
(c)(2)(i) an intracellular portion of CD3ε that comprises an Intracellular Tyrosine-based Activation Motif (ITAM) and a Proline Rich Sequence (PRS), but does not comprise a Basic Residue Sequence (BRS), and
(c)(2)(ii) an endodomain or effector domain from CD3ζ, or a functional portion or variant thereof.
